SEQUENCE LISTING (1 sequence)

SEQ ID NO: 1 - protein, 29 aa, synthetic construct
  REGION 1..29: note = Synthetic Peptide
  MOD_RES 2: note = Aib
  SITE 16: note = Glu side chain lactam linkage to Lys
  SITE 16..20: note = cyclic
  CHAIN 17: note = Z17CO2H
  SITE 20: note = Lys side chain lactam linkage to Glu
HXQGTFTSDYSKYLDEKAAKEFIQWLLQT

(19) United States
(12) Patent Application Publication
(10) Pub. No.: US 2024/0269238 A1
(43) Pub. Date: Aug. 15, 2024

(54) Therapeutic Regimens and Methods for Reducing Body Weight in a Subject with Fatty Liver Disease using a GLP-1R and GCGR Agonist

(71) Applicant: Spitfire Pharma LLC, Gaithersburg, MD (US)

(72) Inventors: Sarah K. Browne, Gaithersburg, MD (US); Matthew Scott Harris, Gaithersburg, MD (US); John Joseph Suschak, III, Damascus, MD (US); Bertrand Victor Gilbert Georges, London (GB); M. Scot Roberts, Myersville, MD (US)

(73) Assignee: Spitfire Pharma LLC, Gaithersburg, MD (US)

(21) Appl. No.: 18/467,608

(22) Filed: Sep. 14, 2023

Related U.S. Application Data
(60) Provisional application No. 63/490,465, filed on Mar. 15, 2023.
(60) Provisional application No. 63/476,370, filed on Dec. 20, 2022.
(60) Provisional application No. 63/422,981, filed on Nov. 5, 2022.
(60) Provisional application No. 63/406,681, filed on Sep. 14, 2022.

(51) Int. Cl. A61K 38/26; A61K 9/08; A61K 47/18; A61K 47/26; A61P 3/04

(52) U.S. Cl. CPC A61K 38/26; A61K 9/08; A61K 47/183; A61K 47/26; A61P 3/04

(57) ABSTRACT

This disclosure relates to the once weekly dosing regimen of a dual GLP-1R and GCGR agonist, formulations, and methods of using the same for inducing weight loss in a human being with fatty liver disease, wherein the human may or may not have other comorbidities such as type 2 diabetes and/or cardiovascular disease/disorder. The dual GLP-1R and GCGR agonist includes the peptide product of SEQ ID NO. 1 (pemvidutide).

### RELATED APPLICATIONS

This application claims priority to U.S. Provisional Appln. Ser. No. 63/406,681 filed 14 Sep. 2022; U.S. Provisional Appln. Ser. No. 63/422,981 filed 5 Nov. 2022; U.S. Provisional Appln. Ser. No. 63/476,370 filed 20 Dec. 2022 and U.S. Provisional Appln. Ser. No. 63/490,465 filed 15 Mar. 2023, each of which is hereby incorporated into this application in their entireties.

### SEQUENCE LISTING

The instant application contains a Sequence Listing which has been submitted electronically in ASCII format via EFS-Web and hereby incorporated by reference in its entirety. Said ASCII copy, created on 13 Apr. 2024, is named MED012US1_Corr.xml and is 4335 bytes in size.

### FIELD OF THE DISCLOSURE

This disclosure relates to the use of the GLP-1R and GCGR agonist Pemvidutide (ALT-801), a composition comprising SEQ ID NO.: 1 in certain dosing regimens for the treatment of obesity with certain comorbidities (e.g., fatty liver disease such as non-alcoholic fatty liver disease (NAFLD) and non-alcoholic steatohepatitis (NASH); type 2 diabetes).

### BACKGROUND OF THE DISCLOSURE

The increasing prevalence of metabolic disorders, including obesity, diabetes mellitus (e.g., type 2 diabetes), non-alcoholic fatty liver disease (NAFLD) and its advanced form, non-alcoholic steatohepatitis (NASH), is a world health crisis of epidemic proportions that is a major contributor to patient morbidity and mortality as well as a major economic burden. Obesity is an important risk factor for type 2 diabetes and NASH, and roughly 90% of patients with type 2 diabetes are overweight or obese. Obesity is a rapidly increasing problem worldwide and currently more than 65% of adults in the U.S. are overweight, and the number of obese people doubles yearly.

In the United States (US), NASH has become the leading cause of end-stage liver disease or liver transplantation. Obesity is the core driver of NASH and weight loss results in reduction in liver fat and NASH improvement. More than 80% of individuals with NASH are overweight or obese, and with no currently available US Food and Drug Administration (FDA)-approved pharmacologic options for inducing weight loss, therapy has largely been based on lifestyle interventions directed at achieving weight loss. However, it is difficult to attain and maintain long-term weight loss with lifestyle changes alone.

Glucagon-like peptide-1 receptor agonists (GLP-1RA) are associated with modest degrees of weight loss at approved doses, and these agents have emerged as a treatment option for patients with NASH. In a recent clinical trial, liraglutide, a daily GLP-1RA, was associated with resolution of NASH, with a trend towards improvement of liver fibrosis. However, patients lost only 5.5% body weight. In one study, 10% or greater weight loss was required for optimal NASH resolution. Higher levels of weight loss have also been associated with lower incidences of cardiovascular disease and non-hepatic malignancies, which represent the most serious co-morbidities facing NASH patients.

GLP-1RAs exert central effects on appetite and food intake, while GCGR agonists (GCGRAs) drive increased energy expenditure in animal models and humans. The effects of GCGRAs and GLP-1RA have been shown to be synergistic in driving greater degrees of weight loss compared to a GLP-1RA alone. GCGRAs also enhance lipolysis and suppress liver fat synthesis, providing an additional pathway for liver fat reduction and NASH resolution.

Dual agonists combine GCGRAs with GLP-1RA in the same molecule. In obese non-human primates, chronic administration of a GLP-1R/GCGR dual agonist reduced body weight and improved glucose tolerance to a greater degree compared to a GLP-IRA mono-agonist. Clinical studies of cotadutide, a GLP-1/GCGR dual agonist with a 5:1 bias of GLP-1 to glucagon activity, demonstrated an impressive 39% reduction in liver fat content in just 6 weeks and greater improvement in NASH-related alanine aminotransferase (ALT) reduction than liraglutide alone. However, the degree of weight loss over 26 weeks of cotadutide administration was comparable to liraglutide (5.4% vs. 5.5%), suggesting that the 5:1 ratio was acceptable for liver fat reduction but suboptimal for weight reduction. Balanced (1:1) agonism has been shown to be associated with greater weight loss and metabolic effects than biased ratios that favor one agonist over the other. A recent study with JNJ 64565111, a balanced dual agonist, achieved an impressive 8% reduction in body weight in just 12 weeks (NCT03586830).

Unfortunately, GLP-1Ras, and GLP-1R and GLP-1 based dual receptor agonists with bias towards GLP-1, have been associated with high rates of nausea, vomiting and diarrhea. These agents must also be titrated over prolonged periods to reduce side effects, and agents with improved tolerability and dosing regimens are needed. Accordingly, there remains a need for convenient dosing (e.g., weekly instead of daily) with a therapeutic dose to reduce body weight in subjects with fatty liver disease independent of other comorbidities such as type 2 diabetes that does not need to be titrated for long periods of time (e.g., more than 4 weeks) to reach a therapeutic level in the absence of gastrointestinal side effects.

### SUMMARY OF THE DISCLOSURE

Described herein are dual agonist peptides and products thereof (e.g., formulations) and uses of the same for treating metabolic disorders associated with the function of glucagon-like peptide 1 receptor (GLP-1R) and glucagon receptor (GCGR) including treatment of obesity and associated co-moieties such as fatty liver disease.

In certain embodiments is provided a method of reducing body weight in a human being with fatty liver disease, wherein the method comprises administering pemvidutide once weekly in an amount from at least 1.8 mg up to 2.4 mg to the human being in need thereof; and, wherein the human being may suffer from type 2 diabetes and wherein the fatty liver disease is non-alcoholic fatty liver disease (NAFLD) or non-alcoholic steatohepatitis (NASH). In embodiments, the body weight of the human being is reduced by at least 3%, or by at least 4% from baseline at week 12, and/or after 24 weekly doses. In embodiments, the pemvidutide is administered once weekly in an amount of 1.8 mg, or once weekly in an amount of 2.4 mg. In certain embodiments, a steady state dose is achieved after a dose escalating phase having a duration of about 2 weeks, about 3 weeks or about 4 weeks. In certain embodiments, the human being suffers from type 2 diabetes. In alternative embodiments, the human being does not suffer from type 2 diabetes. In embodiments, the human being has a body mass index (BMI kg/m²) of at least 27, or a body mass index (BMI kg/m²) of 30 or greater. In certain embodiments, the human being has a level of liver fat as measured by MRI-PDFF (magnetic resonance imaging-proton density fat fraction) of 10% or greater.

In certain embodiments, the methods provided herein induce an absolute reduction in liver fat as measured by MRI-PDFF of about 8% to about 15%, or greater, after 12 weeks and/or after 24 weekly doses of the once weekly dosing. In certain other embodiments, the methods provided herein induce a relative reduction in liver fat as measured by MRI-PDFF compared to baseline of about 40% to about 70% after 12 weeks and/or after 24 weekly doses of the once weekly dosing.

Other aspects of this disclosure are also contemplated as will be understood from the same by those of ordinary skill in the art.

### BRIEF DESCRIPTION OF THE DRAWINGS

FIG. 1 shows four treatment arms wherein subjects (overweight/obese and NAFLD) were randomized into one of the four arms, and the subjects were stratified by the presence or absence of type 2 diabetes (T2D). Two treatment arms (1.2 mg and 1.8 mg dose) had no dose escalation, whereas the 2.4 mg dose arm had a rapid 4-week dose titration. The study had no caloric restriction or lifestyle intervention for the subjects.

FIG. 2 shows liver fat content reduction as determined by MRI-PDFF at week 12, both as an absolute reduction and a relative reduction.

FIG. 3 shows liver fat content reduction by MRI-PDFF at week 12 (Responder Analysis), wherein data is presented as those proportion of patients that demonstrated a 30% liver fat reduction, a 50% liver fat reduction and the normalization group.

FIG. 4 shows weight loss at week 12 in non-diabetic and diabetic (type 2 diabetic) subjects.

FIG. 5 shows ALT reduction at week 12.

FIG. 6 shows Iron-corrected T1 (cT1) responder analyses at week 12

FIG. 7 shows the adverse events (AEs) recorded in this study with very low occurrence for all three doses. The 2.4 mg dose is compared to 2.4 mg* which are the recorded adverse events from an earlier study wherein no dose titration was given in that study. Pemvidutide in this study and in each treatment arm was well tolerated with a reduction in AEs as compared to no dose titration for the 2.4 mg dose. See WO 2022/125598.

FIG. 8 illustrates lipid metabolite extraction, UHPLC-MS analysis, and data processing.

FIG. 9 shows lipoprotein changes in obese/overweight subjects treated with Pemvidutide 1.2 mg (n=6), 1.8 mg (n=9), 2.4 mg (n=9) or placebo (n=10). Day 43 vs. Day −1, Day 84 vs. Day −1. The color code represents the log 2 (robust fold-change) with blue colors denoting reduced lipoproteins (negative fold-changes) and red colors denoting increased lipoproteins (positive fold-changes).

FIG. 10 shows the lipidomic signatures in obese/overweight subjects treated with Pemvidutide 1.2 mg (n=6), 1.8 mg (n=9), 2.4 mg (n=9) or placebo (n=10) comparing changes at Day 84 vs Day −1. Results are presented as log 2 pairwise fold change with blue colors denoting reduced metabolites (negative fold-changes) and red colors denoting increased metabolites (positive fold-changes). Grey/black bars indicate significant p-values of Wilcoxon test (light grey, p<0.05; dark grey, p<0.01; black, p<0.001). (Definitions: PE=Phosphatidylethanolamine; PC=Phosphatidylcholine; PI=Phosphatidylinositol).

FIG. 11 illustrates volcano plots of changes in known atherogenic lipid species following Pemvidutide treatment at 1.8 mg. Y-axis is Log₁₀ of the statistical changes based on Student's T test with horizontal dotted line corresponding to p value threshold of 0.01. X-axis represents the Log₂ fold-change with dotted lines corresponding to arbitrary change threshold of ±0.75.

FIG. 12A illustrates significant weight loss induced by Pemvidutide administration as compared to placebo.

FIG. 12B illustrates greater than or equal to 5% or 10% weight loss induced by Pemvidutide administration as compared to placebo.

FIG. 13 shows the extension study disposition as described in Example 3.

FIG. 14 details baseline characteristics of the participants in study of Example 3.

FIG. 15 shows a robust reduction in liver fat content as determined by MRI-PDFF at week 24 for all doses of Pemvidutide administered (1.2 mg, 1.8 mg, and 2.4 mg per week) as compared to placebo and baseline at week 0 in both absolute and relative % reduction in liver fat.

FIG. 16 shows a robust reduction in liver fat content in responders at week 24 as determined by MRI-PDFF, wherein significant (p<0.001 or p<0.0001 as shown therein) reductions in liver fat content in responders for all doses of Pemvidutide administered (1.2 mg, 1.8 mg, and 2.4 mg per week) as compared to placebo and baseline week 0.

FIG. 17 shows marked defatting of the liver as determined by MRI-PDFF at Week 24 (Pemvidutide, 1.8 mg dose). The reduction in liver volume in this scan of an exemplary subject to whom Pemvidutide was administered was significant (32.3% liver fat at baseline as compared to 1.7% liver fat at Week 24).

FIG. 18 shows a robust reduction in liver volume as determined by MRI-PDFF at Week 24, measured as both absolute reduction and relative reduction. As shown therein, e.g., significant (p<0.05 or p<0.001 as indicated therein) reductions in liver fat content for all doses of Pemvidutide administered (1.2 mg, 1.8 mg, and 2.4 mg per week) as compared to placebo and baseline week 0 were observed.

FIG. 19 shows a robust reduction of ALT levels, a biomarker of liver inflammation, at Week 24, in all subjects and in subjects with a baseline ALT ≥30 IU/L. As shown therein, e.g., significant (p<0.001) reductions in ALT for all doses of Pemvidutide administered (1.2 mg, 1.8 mg, and 2.4 mg per week) as compared to placebo and baseline week 0 were observed.

FIG. 20 shows high rates of cT1 response at Week 24, wherein a response is defined as an 80 MS reduction in CT1 as compared to baseline (Week 0), which has been associated with a two (2) point reduction of NASH Activity Score (NAS) (Dennis A., Front. Endocrinol., 2021). As shown therein, e.g., significant (p<0.05 or p<0.005 as indicated therein) cT1 responses for all doses of Pemvidutide administered (1.2 mg, 1.8 mg, and 2.4 mg per week) as compared to placebo and baseline week 0 were observed.

FIG. 21 shows continued weight loss at Week 24 with significant (p<0.005 or p<0.001 as indicated therein) reductions in weight for all doses of Pemvidutide administered (1.2 mg, 1.8 mg, and 2.4 mg per week) as compared to placebo and baseline week 0 were observed in all subjects (non-diabetic patients and diabetic patients). This shows weight loss in a human being with fatty liver disease (non-alcoholic fatty liver disease (NAFLD) or non-alcoholic steatohepatitis (NASH)) in both diabetic and non-diabetic subjects.

FIG. 22 shows improvements in serum lipids at Week 24 as compared to baseline (Week 0) for all doses of Pemvidutide administered (1.2 mg, 1.8 mg, and 2.4 mg per week).

FIG. 23 shows all doses of Pemvidutide tested induced improvements in blood pressure without clinically meaningful increases in heart rate at Week 24 compared to placebo and baseline (week 0) and that systolic blood pressure reduction was significant (p<0.05) at the 2.4 mg per week dose.

FIG. 24 shows the safety overview of this trial with adverse events over 24 weeks.

FIG. 25 shows improvement in glycemic control in diabetic patients at Week 24 for all doses of Pemvidutide administered (1.2 mg, 1.8 mg, and 2.4 mg per week) as compared to placebo and baseline week 0.

FIG. 26 shows baseline characteristics of study participants of Phase II Study. See Example 4.

FIG. 27 shows body weight loss expressed as mean percentage change of baseline body weight achieved over 24-week treatment with Pemvidutide at 1.2 mg, 1.8 mg and 2.4 mg doses and placebo in all evaluated subjects.

FIG. 28 shows body weight loss expressed as mean percentage change of baseline body weight achieved over 24-week treatment with Pemvidutide at 1.2 mg, 1.8 mg and 2.4 mg doses and placebo in a subgroup of subjects with baseline body weight ≤115 kg.

FIG. 29 shows percentage of subjects in each group achieving ≥5%, ≥10% or ≥15% body weight loss expressed as percentage of baseline body weight after 24-week treatment with Pemvidutide at 1.2 mg, 1.8 mg and 2.4 mg doses and placebo.

FIG. 30 shows body weight loss expressed as mean percentage change of baseline body weight achieved in Hispanic subjects versus non-Hispanic subjects after 24-week treatment with Pemvidutide at 1.2 mg, 1.8 mg and 2.4 mg doses and placebo.

FIG. 31 show systolic and diastolic blood pressure expressed as mean percentage change from baseline (A) and Heart rate expressed as mean percentage change from baseline (B) after 24-week treatment with Pemvidutide at 1.2 mg, 1.8 mg and 2.4 mg doses and placebo.

FIG. 32 shows serum lipids at week 24 as compared to baseline for all doses of Pemvidutide administered (1.2 mg, 1.8 mg, and 2.4 mg).

FIG. 33 shows a significant reduction in waist circumference at week 24 for all doses of Pemvidutide administered (1.2 mg, 1.8 mg, and 2.4 mg per week). See Example 4.

### DETAILED DESCRIPTION OF THE DISCLOSURE

This disclosure relates to a dual agonist peptide(s) as well as pharmaceutical dosage formulations comprising, and methods for using, the same. The dual agonist peptides have affinity for, and in preferred embodiments about equal affinity for, glucagon-like peptide 1 receptor (GLP-1R) and glucagon receptor (GCGR), as may be determined using a cellular assay. In some embodiments, this disclosure provides pharmaceutical dosage formulations configured to induce weight loss in a human being with fatty liver disease. In some embodiments, the human being may, or may not, suffer from type 2 diabetes. In some embodiments, the disclosure provides pharmaceutical dosage formulations configured to reduce pathogenic plasma lipid mediators. As used herein, “pathogenic” serum lipid mediators, included but are not limited to the following reactive lipid species: malondialdehyde (MDA), isolevuglandins (IsoLG), methyglyoxal (MGO), 4-oxononenal (ONE), and 4-hydroxynonenal (HNE). Oxidized phospholipids include 1-palmitoyl-2-oxovaleroyl-sn-glycero-3-phosphorylcholine (POVPC), 1-O-alkyl-2-azelaoyl-sn-glycero-3-phophorylcholine (azPAF), 1-(Palmitoyl)-2-(5-keto-6-octene-dioyl) phosphatidylcholine (KOdiA-PC), 1-palmitoyl-2-F2-isoprostane-sn-glycero-3-phosphocholine (F2IsoP-PC), and 1-palmitoyl-2-(5,6)-epoxyisoprostane E2-sn-glycero-3-phosphocholine (PEIPC).

In some embodiments, this disclosure provides pharmaceutical dosage formulations configured to induce weight loss including for treatment of chronic weight management and associated comorbidities. In some embodiments, the disclosure provides pharmaceutical dosage formulations configured to induce weight loss and reduce pathogenic serum lipid mediators for treatment of obesity (e.g. chromic weight management) and/or treatment for cardiovascular (CV) associated risk factors. In some embodiments, this disclosure provides peptide-based dual GLP-1/glucagon receptor agonists designed to treat the underlying metabolic dysfunction that leads to non-alcoholic steatohepatitis (NASH).

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of obesity” means an activity that alleviates or ameliorates any of the primary phenomena or secondary symptoms associated with the obesity or other conditions described herein, including, but not limited to fatty liver disease (FDL) (e.g., NASH and NAFLD) and type 2 diabetes.

In embodiments, provided herein is a method a method of reducing body weight in a human being with fatty liver disease, wherein the method comprises administering pemvidutide once weekly in an amount from at least 1.8 mg up to 2.4 mg to the human being in need thereof; and, wherein the human being may suffer from type 2 diabetes and wherein the fatty liver disease is non-alcoholic fatty liver disease (NAFLD) or non-alcoholic steatohepatitis (NASH). pemvidutide, also referred to herein as ALT-801, is a composition comprising a synthetic peptide (SEQ ID NO: 1) composed of naturally occurring amino acids and is a chimeric analog of the two native hormones GLP-1 and glucagon, with predominantly glucagon residues in the N-terminus and GLP-1 residues in the C-terminus. See U.S. Pat. No. 9,856,306, incorporated herein by reference. ALT-801 also incorporates one nonproteinogenic amino acid, 2-aminoisobutyric acid, an amino acid side chain amide linkage (lactam bridge), and a surfactant side chain composed of a glucuronic acid linked to an octadecanoic fatty acid side chain. The surfactant side chain appears to slow entry in the circulation and may form micelles after subcutaneous (SC) injection. The lower maximal concentration (Cₘₐₓ) associated with slower entry may result in fewer GI side effects and better tolerability. This latter feature also enhances binding to plasma proteins and improves the metabolic stability, extending the half-life (t_{1/2}). The design of ALT-801 provides a co-agonist with equipotent (1:1) activity at both receptors of approximately 40 pM and 100% activity. Compositions comprising SEQ ID NO: 1 have been administered to human beings across a variety of doses and found not to induce side effects such as nausea, vomiting, diarrhea, abdominal pain and/or constipation using standard techniques. See US Patent Publ. No. 2021/0290732 and PCT Publication No. WO 2022/125598, each incorporated herein by reference.

ALT-801 (SEQ ID NO: 1 (also referred to herein as pemvidutide)) has the following amino acid sequence:
¹His-²Aib-³Gln-⁴Gly-⁵Thr-⁶Phe-⁷Thr-⁸Ser-⁹Asp-¹⁰Tyr-¹¹Ser-¹²Lys-¹³Tyr-¹⁴Leu-¹⁵Asp-¹⁶Glu*-¹⁷Lys^{#}-¹⁸Ala-¹⁹Ala-²⁰Lys*-²¹Glu-²²Phe-²³Ile-²⁴Gln-²⁵Trp-²⁶Leu-²⁷Leu-²⁸Gln-²⁹Thr-NH₂,
   where * indicates a lactam bridge is formed between Glu16 and Lys 20, and 17Lys #indicates the attachment site for glucuronic acid C-18*(Z17COOH). Illustrated differently, SEQ ID NO: 1 (i.e., ALT-801 or pemvidutide) is a peptide amide consisting of 29 amino acid residues and a glucuronic acid/C₁₈ diacid moiety attached to ¹⁷Lys, in which the side-chains of ¹⁶Glu and ²⁰Lys forming an intramolecular cycle as shown below:

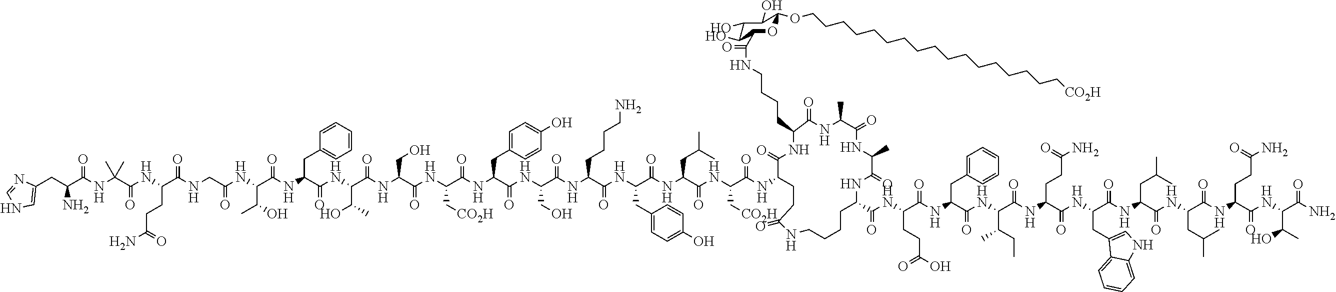

In embodiments provided herein is a pharmaceutical formulation of SEQ ID NO: 1 in an aqueous buffer solution, referred to herein as ALT-801 (and pemvidutide). The dual agonist peptide products herein, including SEQ ID NO: 1, comprise an amino acid side chain amide linkage (lactam bridge), and a surfactant side chain composed of a glucuronic acid linked to a fatty acid side chain. The side chain, a surfactant comprised of a hydrophilic saccharide group covalently attached to the peptide via a linker amino acid, and a hydrophobic alkyl chain portion. The synthesis of SEQ ID NO. 1 is described in U.S. Pat. No. 11,541,028 B2, which is incorporated by reference in its entirety into this disclosure. In some embodiments, the dual agonist peptides can include one or more conservatively substituted amino acids as described herein. In preferred embodiments, SEQ ID NO: 1 can include one or more conservatively substituted amino acids, but preferably not at amino acid residues 16, 17, or 20.

A “peptide” (e.g., dual agonist peptide) comprises two or more natural or/and unnatural amino acid residues linked typically via peptide bonds. Such amino acids can include naturally occurring structural variants, naturally occurring non-proteinogenic amino acids, or/and synthetic non-naturally occurring analogs of natural amino acids. The terms “peptide” and “polypeptide” are used interchangeably herein. Peptides include short peptides (about 2-20 amino acids), medium-length peptides (about 21-50 amino acids) and long peptides (>about 50 amino acids, which can also be called “proteins”). In some embodiments, a peptide product comprises a surfactant moiety covalently and stably attached to a peptide of no more than about 50, 40 or 30 amino acids. Synthetic peptides can be synthesized using an automated peptide synthesizer, for example. Peptides can also be produced recombinantly in cells expressing nucleic acid sequences that encode the peptides. Conventional notation is used herein to portray peptide sequences: the left-hand end of a peptide sequence is the amino (N)-terminus, and the right-hand end of a peptide sequence is the carboxyl (C)-terminus. Standard one-letter and three-letter abbreviations for the common amino acids are used herein. Although the abbreviations used in the amino acid sequences disclosed herein represent L-amino acids unless otherwise designated as D- or DL- or the amino acid is achiral, the counterpart D-isomer generally can be used at any position (e.g., to resist proteolytic degradation). Abbreviations for other amino acids used herein include: Aib=a-aminoisobutyric acid (or 2-methylalanine or Ca-methylalanine); Xaa: any amino acid, typically specifically defined within a formula. Abbreviations for other amino acids that can be used as described herein include: Ac3c=1-aminocyclopropane-1-carboxylic acid; Ac4c=1-aminocyclobutane-1-carboxylic acid; Ac5c=1-aminocyclopentane-1-carboxylic acid; Ac6c=1-aminocyclohexane-1-carboxylic acid; Aib=alpha-aminoisobutyric acid (or 2-methylalanine or Calpha-methylalanine); Bip=3-(biphenyl-4-yl)alanine; Bip2Et=3-(2′-ethylbiphenyl-4-yl)alanine; Bip2EtMeO=3-(2′-ethyl-4′-methoxybiphenyl-4-yl)alanine; Cit=citrulline; Deg=2,2-diethylglycine; Dmt=(2,6-dimethyl)tyrosine; 2FPhe=(2-fluorophenyl)alanine; 2FMePhe or 2FaMePhe=Ca-methyl-(2-fluorophenyl)alanine; hArg=homoarginine; MeLys or aMeLys=Ca-methyllysine; MePhe or aMePhe=Ca-methylphenylalanine; MePro or aMePro=Ca-methylproline; Nal1 or Nal(1)=3-(1-naphthyl)alanine; Nal2 or Nal(2)=3-(2-naphthyl)alanine; Nle norleucine; Om=ornithine; and Tmp=(2,4,6-trimethylphenyl)alanine; 1,2,3,4-tetrahydroisoquinoline-3-carboxylic acid (Tic) and a Tic-Phe dipeptide moiety with a reduced amide bond between the residues (designated as Tic-Ψ[CFl2-NFl]-Ψ-Phe) have the following structures:

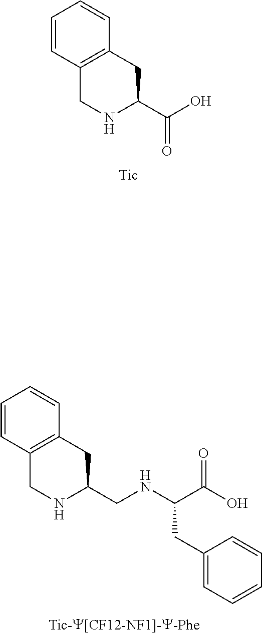 [SMILES: O=C(O)[C@@H]1Cc2ccccc2CN1 ; O=C(O)[C@H](Cc1ccccc1)NC[C@@H]1Cc2ccccc2CN1]

Unless specifically stated otherwise or the context clearly indicates otherwise, the disclosure encompasses any and all forms of a dual agonist peptide that may be produced, whether the dual agonist peptide is produced synthetically (e.g., using a peptide synthesizer) or by a cell (e.g., by recombinant production). Such forms of a dual agonist peptide can include one or more modifications that may be made during the course of synthetic or cellular production of the peptide, such as one or more post-translational modifications, whether or not the one or more modifications are deliberate. A dual agonist peptide can have the same type of modification at two or more different places, or/and can have two or more different types of modifications. Modifications that may be made during the course of synthetic or cellular production of a dual agonist peptide, including chemical and post-translational modifications, include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. A dual agonist peptide can have one or more modifications anywhere, such as the N-terminus, the C-terminus, one or more amino acid side chains, or the dual agonist peptide backbone, or any combination thereof. In some embodiments, a dual agonist peptide is acetylated at the N-terminus or/and has a carboxamide (—CONH₂) group at the C-terminus, which can increase the stability of the dual agonist peptide.

Potential modifications of a dual agonist peptide also include deletion of one or more amino acids, addition/insertion of one or more natural or/and unnatural amino acids, or substitution with one or more natural or/and unnatural amino acids, or any combination or all thereof. A substitution can be conservative or non-conservative. Such modifications may be deliberate, such as via site-directed mutagenesis or in the chemical synthesis of a dual agonist peptide, or may be accidental, such as via mutations arising in the host cell that produces the dual agonist peptide or via errors due to PCR amplification. An unnatural amino acid can have the same chemical structure as the counterpart natural amino acid but have the D stereochemistry, or it can have a different chemical structure and the D or L stereochemistry. Unnatural amino acids can be utilized, e.g., to promote a-helix formation or/and to increase the stability of the dual agonist peptide (e.g., to resist proteolytic degradation). A dual agonist peptide having one or more modifications relative to a reference dual agonist peptide may be called an “analog” or “variant” of the reference dual agonist peptide as appropriate. An “analog” typically retains one or more essential properties (e.g., receptor binding, activation of a receptor or enzyme, inhibition of a receptor or enzyme, or other biological activity) of the reference dual agonist peptide. A “variant” may or may not retain the biological activity of the reference dual agonist peptide, or/and may have a different biological activity. It is preferred that such a variant maintain its ability to act as an agonist of GLP-1R and GCGR, and in more preferred embodiments, has about equal affinity for GLP-1R and GCGR. In some embodiments, an analog or variant of a reference peptide has a different amino acid sequence than the reference dual agonist peptide.

The term “conservative substitution” refers to substitution of an amino acid in a dual agonist peptide with a functionally, structurally or chemically similar natural or unnatural amino acid. In certain embodiments, the following groups each contain natural amino acids that are conservative substitutions for one another: 1) Glycine (Gly/G), Alanine (Ala/A); 2) Isoleucine (Ile/I), Leucine (Leu/L), Methionine (Met/M), Valine (Val/V); 3) Phenylalanine (Phe/F), Tyrosine (Tyr/Y), Tryptophan (Trp/W); 4) Serine (Ser/S), Threonine (Thr/T), Cysteine (Cys/C); 5) Asparagine (Asn/N), Glutamine (Gln/Q); 6) Aspartic acid (Asp/D), Glutamic acid (Glu/E); and, 7) Arginine (Arg/R), Lysine (Lys/K), Histidine (His/H). In further embodiments, the following groups each contain natural amino acids that are conservative substitutions for one another: 1) non-polar: Ala, Val, Leu, Ile, Met, Pro (proline/P), Phe, Trp; 2) hydrophobic: Val, Leu, Ile, Phe, Trp; 3) aliphatic: Ala, Val, Leu, Ile; 4) aromatic: Phe, Tyr, Trp, His; 5) uncharged polar or hydrophilic: Gly, Ala, Pro, Ser, Thr, Cys, Asn, Gln, Tyr; 6) aliphatic hydroxyl- or sulfhydryl-containing: Ser, Thr, Cys; 7) amide-containing: Asn, Gln; 8) acidic: Asp, Glu; 9) basic: Lys, Arg, His; and, 10) small: Gly, Ala, Ser, Cys. In other embodiments, amino acids may be grouped as conservative substitutions as set out below: 1) hydrophobic: Val, Leu, Ile, Met, Phe, Trp; 2) aromatic: Phe, Tyr, Trp, His; 3) neutral hydrophilic: Gly, Ala, Pro, Ser, Thr, Cys, Asn, Gln; 4) acidic: Asp, Glu; 5) basic: Lys, Arg, His; and, 6) residues that influence backbone orientation: Pro.

Examples of unnatural or non-proteinogenic amino acids include without limitation alanine analogs (e.g., α-ethylGly [α-aminobutyric acid or Abu], α-n-propylGly [norvaline or Nva], α-tert-butylGly [Tbg], α-vinyl Gly [Vg or Vlg], α-allylGly [Alg], α-propargylGly [Prg], 3-cyclopropylAla [Cpa] and Aib), leucine analogs (e.g., nor-leucine, Nle), proline analogs (e.g., (α-MePro), phenylalanine analogs (e.g., Phe(2-F), Phe(2-Me), Tmp, Bip, Bip(2′-Et-4′-OMe), Nal1, Nal2, Tic, α-MePhe, α-MePhe(2-F) and (α-MePhe(2-Me)), tyrosine analogs (e.g., Dmt and (α-MeTyr), serine analogs (e.g., homoserine [isothreonine or hSer]), glutamine analogs (e.g., Cit), arginine analogs (e.g., hArg, N,N′-g-dialkyl-hArg), lysine analogs (e.g, homolysine [hLys], Orn and (α-MeLys), α, α-disubstituted amino acids (e.g., Aib, α, α-diethylGly [Deg], (α-cyclohexylAla [2-Cha], Ac3c, Ac4c, Ac5c and Ac6c), and other unnatural amino acids disclosed in A. Santoprete et al., Pept. Sci., 17:270-280 (2011). α,α-Di-substituted amino acids can provide conformational restraint or/and a-helix stabilization. A reduced amide bond between two residues (as in, e.g., Tic-Ψ[CFl2-NFl]-Ψ-Phe) increases protease resistance and may also, e.g., alter receptor binding. The disclosure encompasses all pharmaceutically acceptable salts of dual agonist peptides, including those with a positive net charge, those with a negative net charge, and those with no net charge.

An “alkyl” group refers to an aliphatic hydrocarbon group. An alkyl group can be saturated or unsaturated, and can be straight-chain (linear), branched or cyclic. In some embodiments, an alkyl group is not cyclic. In some embodiments, an alkyl group contains 1-30, 6-30, 6-20 or 8-20 carbon atoms. A “substituted” alkyl group is substituted with one or more substituents. In some embodiments, the one or more substituents are independently selected from halogens, nitro, cyano, oxo, hydroxy, alkoxy, haloalkoxy, aryloxy, thiol, alkylthio, arylthio, alkylsulfoxide, arylsulfoxide, alkylsulfone, arylsulfone, amino, alkylamino, dialkylamino, arylamino, alkoyl, carboxyl, carboxylate, esters, amides, carbonates, carbamates, ureas, alkyl, haloalkyl, fluoroalkyl, aralkyl, alkyl chains containing an acyl group, heteroalkyl, heteroali-cyclic, aryl, alkoxyaryl, heteroaryl, hydrophobic natural compounds (e.g., steroids), and the like. In some embodiments, an alkyl group as a substituent is linear or branched Ci-C₆ alkyl, which can be called “lower alkyl”. Non-limiting examples of lower alkyl groups include methyl, ethyl, propyl (including n-propyl and isopropyl), butyl (including all isomeric forms, such as n-butyl, isobutyl, sec-butyl and/er/-butyl), pentyl (including all isomeric forms, such as n-pentyl), and hexyl (including all isomeric forms, such as n-hexyl). In some embodiments, an alkyl group is attached to the Na-atom of a residue (e.g., Tyr or Dmt) of a peptide. In certain embodiments, an N-alkyl group is straight or branched C1-Cio alkyl, or aryl-substituted alkyl such as benzyl, phenylethyl or the like. One or two alkyl groups can be attached to the Na-atom of the N-terminal residue. In some embodiments, an alkyl group is a 1-alkyl group that is attached to the C-1 position of a saccharide (e.g., glucose) via a glycosidic bond (e.g., an O-, S-, N- or C-glycosidic bond). In some embodiments, such a 1-alkyl group is an unsubstituted or substituted C1-C30, C₆-C₃₀, C₆-C₂₀ or C₅-C₂₀ alkyl group. In some embodiments, an alkyl group (e.g., a 1-alkyl group) is substituted with one or more (e.g., 2 or 3) groups independently selected from aryl, —OH, —OR¹, —SH, —SR¹, —NH₂, —NHR¹, —N(R¹)₂, oxo (═O), —C(═O)R², carboxyl (—CO₂H), carboxylate (—CO₂—), —C(═O)OR¹, —OC(═O)R³, —C(═O)N(R¹)₂, —NR⁴C(═O)R³, —OC(═O)OR⁵, —OC(═O)N(R¹)₂, —NR⁴C(═O)OR⁵, and —NR⁴C(═O)N(R¹)₂, wherein: R¹ at each occurrence independently is hydrogen, alkyl or aryl, or both occurrences of R¹ and the nitrogen atom to which they are connected form a heterocyclyl or heteroaryl ring; R² at each occurrence independently is alkyl, heterocyclyl, aryl or heteroaryl; R³ at each occurrence independently is hydrogen, alkyl, heterocyclyl, aryl or heteroaryl; R⁴ at each occurrence independently is hydrogen or alkyl; and, R⁵ at each occurrence independently is alkyl or aryl. In some embodiments, an alkyl group (e.g., a 1-alkyl group) is internally or/and terminally substituted with a carboxyl/carboxylate group, an aryl group or an —O-aryl group. In certain embodiments, an alkyl group (e.g., a 1-alkyl group) is substituted with a carboxyl or carboxylate group at the distal end of the alkyl group. In further embodiments, an alkyl group (e.g., a 1-alkyl group) is substituted with an aryl group at the distal end of the alkyl group. In other embodiments, an alkyl group (e.g., a 1-alkyl group) is substituted with an —O-aryl group at the distal end of the alkyl group. The terms “halogen”, “halide” and “halo” refer to fluoride, chloride, bromide and iodide. The term “acyl” refers to —C(═O)R, where R is an aliphatic group that can be saturated or unsaturated, and can be linear, branched or cyclic. In certain embodiments, R contains 1-20, 1-10 or 1-6 carbon atoms. An acyl group can optionally be substituted with one or more groups, such as halogens, oxo, hydroxyl, alkoxy, thiol, alkylthio, amino, alkylamino, dialkylamino, cycloalkyl, aryl, acyl, carboxyl, esters, amides, hydrophobic natural compounds (e.g., steroids), and the like. The terms “heterocyclyl” and “heterocyclic” refer to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. In certain embodiments, a heterocyclyl or heterocyclic group has from 3 to 15, or 3 to 12, or 3 to 10, or 3 to 8, or 3 to 6 ring atoms. Heterocyclyl or heterocyclic groups include without limitation aziridinyl, azetidinyl, pyrrolidinyl, piperidinyl, morpholinyl, piperazinyl, azepanyl, azocanyl, oxiranyl, oxetanyl, tetrahydrofuranyl (oxolanyl), tetrahydropyranyl, oxepanyl and oxocanyl. The term “aryl” refers to a monocyclic aromatic hydrocarbon group or a multicyclic group that contains at least one aromatic hydrocarbon ring. In certain embodiments, an aryl group has from 6 to 15, or 6 to 12, or 6 to 10 ring atoms. Aryl groups include without limitation phenyl, naphthalenyl (naphthyl), fluorenyl, azulenyl, anthryl, phenanthryl, biphenyl and terphenyl. The aromatic hydrocarbon ring of an aryl group may be attached or fused to one or more saturated, partially unsaturated or aromatic rings—e.g., dihydronaphthyl, indenyl, indanyl and tetrahydronaphthyl (tetralinyl). An aryl group can optionally be substituted with one or more (e.g., 2 or 3) substituents independently selected from halogens (including —F and —Cl), cyano, nitro, hydroxyl, alkoxy, thiol, alkylthio, alkylsulfoxide, alkylsulfone, amino, alkylamino, dialkylamino, alkyl, haloalkyl (including fluoroalkyl such as trifluoromethyl), acyl, carboxyl, esters, amides, and the like. The term “heteroaryl” refers to a monocyclic aromatic group or a multicyclic group that contains at least one aromatic ring, wherein at least one aromatic ring contains one or more heteroatoms independently selected from O, N and S. The heteroaromatic ring may be attached or fused to one or more saturated, partially unsaturated or aromatic rings that may contain only carbon atoms or that may contain one or more heteroatoms. In certain embodiments, a heteroaryl group has from 5 to 15, or 5 to 12, or 5 to 10 ring atoms. Monocyclic heteroaryl groups include without limitation pyrrolyl, pyrazolyl, pyrazolinyl, imidazolyl, oxazolyl, isoxazolyl, thiazolyl, thiadiazolyl, isothiazolyl, furanyl, thienyl (thiophenyl), oxadiazolyl, triazolyl, tetrazolyl, pyridyl, pyridonyl, pyrazinyl, pyrimidinyl, pyridazinyl, pyridazinonyl and triazinyl. Non-limiting examples of bicyclic heteroaryl groups include indolyl, benzothiazolyl, benzothiadiazolyl, benzoxazolyl, benzisoxazolyl, benzothienyl (benzothiophenyl), quinolinyl, tetrahydroisoquinolinyl, isoquinolinyl, benzimidazolyl, benzotriazolyl, indolizinyl, benzofuranyl, isobenzofuranyl, chromonyl, coumarinyl, cinnolinyl, quinazolinyl, quinoxalinyl, indazolyl, naphthyridinyl, phthalazinyl, quinazolinyl, purinyl, pyrrol opyridinyl, furopyridinyl, thienopyridinyl, dihydroisoindolyl and tetrahydroquinolinyl.

In some embodiments, for instance, the dual agonist peptides can be associated with a saccharide, such as within a pharmaceutically acceptable composition or lyophilizate. Saccharides include monosaccharides, disaccharides and oligosaccharides (e.g., trisaccharides, tetrasaccharides and so on). A reducing saccharide exists in a ring form and an open-chain form in equilibrium, which generally favors the ring form. A functionalized saccharide of a surfactant moiety has a functional group suitable for forming a stable covalent bond with an amino acid of a dual agonist peptide.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition. In one embodiment, a pharmaceutically acceptable composition in which a dual agonist peptide can be formulated comprises polysorbate 20 (e.g., about 0.050% (w/w)); optionally methylparaben (e.g., about 0.300% (w/w)); arginine (about 0.348% (w/w)), and mannitol (e.g., about 4.260% (w/w)) in distilled (DI) water.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician.

The terms “treat,” “treating” and “treatment” include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition. The term “medical conditions” (or “conditions” for brevity) includes diseases and disorders. The terms “diseases” and “disorders” are used interchangeably herein.

The disclosure also provides pharmaceutical compositions comprising a dual agonist peptide product described herein or a pharmaceutically acceptable salt thereof, and one or more pharmaceutically acceptable carriers or excipients. A pharmaceutical composition contains a therapeutically effective amount of a peptide product or an appropriate fraction thereof. A composition can optionally contain an additional therapeutic agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

In embodiments, a pharmaceutical formulation comprises a peptide product and about 0.02-0.075% (w/w) polysorbate 20, about 0.2-0.5% (w/w) arginine, about 3-6% (w/w) mannitol in deionized water (pH 7.7±0.1); optionally about 0.050% (w/w) polysorbate 20, about 0.348% (w/w) arginine, about 4.260% (w/w) mannitol in deionized water (pH 7.7±0.1). In certain embodiments, a present pharmaceutical formulation comprises SEQ ID NO: 1 and about 0.050% (w/w) polysorbate 20, about 0.348% (w/w) arginine, about 4.260% (w/w) mannitol in deionized water (pH 7.7±0.1). In certain embodiments, a present pharmaceutical formulation comprises SEQ ID NO: 1 and about 0.020% (w/w) polysorbate 20, about 0.348% (w/w) arginine, about 4.260% (w/w) mannitol in deionized water (pH 7.7±0.1). In certain embodiments, the pharmaceutical formulation comprises SEQ ID NO: 1 (ALT-801, pemvidutide) and is configured for subcutaneous (SC) administration of a weekly therapeutic dose.

An appropriate or suitable formulation can depend on various factors, such as the route of administration chosen. Potential routes of administration of a pharmaceutical composition comprising a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository). In certain embodiments, a present dual agonist peptide product is administered parenterally (e.g., subcutaneously, intravenously or intramuscularly). In other embodiments, a peptide product is administered by oral inhalation or nasal inhalation or insufflation. In some embodiments, the carrier is an aqueous-based carrier, such as in a parenteral (e.g., subcutaneous, intravenous or intramuscular) formulation. In other embodiments, the carrier is a nonaqueous-based carrier. In certain embodiments, the nonaqueous-based carrier is a hydrofluoroalkane (HFA) or HFA-like solvent that may comprise sub-micron anhydrous a-lactose or/and other excipients, such as in a formulation for administration by oral inhalation or nasal inhalation or insufflation.

In some embodiments, a peptide product is administered parenterally (e.g., subcutaneously, intravenously or intramuscularly) by injection. Parenteral administration bypasses the strongly acidic environment of the stomach, gastrointestinal (GI) absorption and first-pass metabolism. Excipients and carriers that can be used to prepare parenteral formulations include without limitation solvents (e.g., aqueous solvents such as water, saline, physiological saline, buffered saline [e.g., PBS], balanced salt solutions [e.g., Ringer's BSS] and aqueous dextrose solutions), isotonic/iso-osmotic agents (e.g., salts [e.g., NaCl, KCl and CaCl₂)] and sugars [e.g., sucrose]), buffering agents and pH adjusters (e.g., sodium dihydrogen phosphate [monobasic sodium phosphate]/di sodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Peptide formulations and delivery systems are discussed in, e.g., A. J. Banga, Therapeutic Peptides and Proteins: Formulation, Processing, and Delivery Systems, 3rd Ed., CRC Press (Boca Raton, Florida) (2015). The excipients can optionally include one or more substances that increase peptide stability, increase peptide solubility, inhibit peptide aggregation or reduce solution viscosity, or any combination or all thereof. Such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose}, osmolytes (e.g., trehalose, taurine, amino acids [e.g., glycine, sarcosine, alanine, proline, serine, b-alanine and g-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™F-68], and Genapol® PF-10 and variants thereof). Because such substances increase peptide solubility, they can be used to increase peptide concentration in a formulation. Higher peptide concentration in a formulation is particularly advantageous for subcutaneous administration, which has a limited volume of bolus administration (e.g., <about 1.5 mL). In addition, such substances can be used to stabilize peptides during the preparation, storage and reconstitution of lyophilized peptides. An exemplary parenteral formulation comprises a peptide product, mannitol, methionine, sodium thioglycolate, polysorbate 20, a pH adjuster (e.g., NaOH or/and HCl) and de-ionized water. Excipients of parenteral formulations that would be suitable for use with the dual agonist peptides described herein (e.g., various combinations of excipients including NaCl and the like) are well-known and available to those of ordinary skill in the art.

For parenteral (e.g., subcutaneous, intravenous or intramuscular) administration, a sterile solution or suspension of a peptide product in an aqueous solvent containing one or more excipients can be prepared beforehand and can be provided in, e.g., a pre-filled syringe of a single-use pen or a pen with a dose counter. Alternatively, a peptide product can be dissolved or suspended in an aqueous solvent that can optionally contain one or more excipients prior to lyophilization (freeze-drying). Shortly prior to parenteral administration, the lyophilized peptide product stored in a suitable container (e.g., a vial) can be reconstituted with, e.g., sterile water that can optionally contain one or more excipients. In other embodiments, an agonist peptide product is administered intranasally. The nasal mucosa provides a big surface area, a porous endothelium, a highly vascular subepithelial layer and a high absorption rate, and hence allows for high bioavailability. An intranasal formulation can comprise a peptide product along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump. Table 2 shows exemplary excipients of nasal-spray formulations.

TABLE 1
Exemplary excipients and carriers
of nasal and pulmonary formulations

| Dosage | |
|---|---|
| Form | Ingredients in Addition to a Peptide Product |
| nasal spray | microcrystalline cellulose, sodium carboxymethylcellulose, |
| | dextrose, water, and optionally a pH adjuster (e.g., HCl) |
| nasal spray | microcrystalline cellulose, carboxymethyl cellulose sodium, |
| | dextrose, polysorbate 80, disodium edetate, potassium |
| | sorbate, a pH adjuster (e.g., HCl), water, and optionally |
| | an alcohol (e.g., ethanol) |
| nasal spray | microcrystalline cellulose, carboxymethyl cellulose sodium, |
| | dextrose, polysorbate 80, benzalkonium chloride, |
| | phenylethyl alcohol, water, and optionally an alcohol |
| | (e.g., ethanol) |
| nasal spray | hypromellose, benzalkonium chloride, NaCl, EDTA, citric |
| | acid, sodium phosphate dibasic, water, and optionally an |
| | alcohol (e.g., ethanol) |
| inhalation | mannitol, glycine, sodium citrate and NaOH |
| (DPI) | |
| inhalation | lactose, starch, a starch derivative (e.g., |
| (DPI) | hydroxypropylmethyl cellulose) or polyvinylpyrrolidine, |
| | and optionally magnesium stearate or/and leucine |
| inhalation | a propellant (e.g., 1,1,1,2-tetrafluoroethane), a surfactant |
| (MDI) | (e.g., lecithin or oleic acid), and a co-solvent (e.g., ethanol) |
| inhalation | polysorbate 80, edetate disodium, sodium chloride, pH |
| (nebulizer) | buffering agents (e.g., citric acid/sodium citrate), and water |

In further embodiments, a peptide product is administered via a pulmonary route, such as by oral inhalation or nasal inhalation. Pulmonary administration of a drug can treat a lung disorder or/and a systemic disorder, as the lungs serve as a portal to the systemic circulation. Advantages of pulmonary drug delivery include, for example: 1) avoidance of first-pass metabolism; 2) fast drug action; 3) large surface area of the alveolar region for absorption, high permeability of the lungs (thin air-blood barrier), and profuse vasculature of the airways; and 4) reduced extracellular enzyme levels compared to the GI tract due to the large alveolar surface area. An advantage of oral inhalation over nasal inhalation includes deeper penetration/deposition of the drug into the lungs, although nasal inhalation can deliver the drug into systemic circulation transmucosally in the nasal cavity as well as in the lungs. Oral or nasal inhalation can be achieved by means of, e.g., a metered-dose inhaler (MDI), a nebulizer or a dry powder inhaler (DPI). For example, a peptide product can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer, although the surfactant moiety of the peptide product can perform functions of a surfactant. For example, an MDI formulation can comprise a peptide product, a propellant (e.g., an HFA such as 1,1,1,2-tetrafluoroethane) and a co-solvent (e.g., an alcohol such as ethanol), and optionally a surfactant (e.g., a fatty acid such as oleic acid). The MDI formulation can optionally contain a dissolved gas (e.g., CO₂). After device actuation, the bursting of CO₂ bubbles within the emitted aerosol droplets breaks up the droplets into smaller droplets, thereby increasing the respirable fraction of drug. As another example, a nebulizer formulation can comprise a peptide product, a chelator or preservative (e.g., edetate disodium), an isotonicity agent (e.g., NaCl), pH buffering agents (e.g., citric acid/sodium citrate) and water, and optionally a surfactant (e.g., a Tween® such as polysorbate 80). The drug can be delivered by means of, e.g., a nebulizer or an MDI with or without a spacer, and the drug dose delivered can be controlled by a metering chamber (nebulizer) or a metering valve (MDI).

Table 1 shows exemplary MDI, nebulizer and DPI formulations. Metered-dose inhalers (also called pressurized metered-dose inhalers [pMDI]) are the most widely used inhalation devices. A metering valve delivers a precise amount of aerosol (e.g., about 20-100 pL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easi breathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer. Nebulizers generate aerosol droplets of about 1-5 microns. They do not require user coordination between device actuation and inhalation, which can significantly affect the amount of drug deposited in the lungs. Compared to MDIs and DP Is, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, MicroAir® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse. For oral or nasal inhalation using a dry powder inhaler (DPI), a peptide product can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or be blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation and determines drug deposition in the lungs. (While a passive DPI requires rapid inspiratory airflow to de agglomerate drug particles, rapid inspiration is not recommended with an MDI or nebulizer, since it creates turbulent airflow and fast velocity which increase drug deposition by impaction in the upper airways.) Compared to an MDI, a DPI (including a breath-activated DPI) may be able to deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SV003 (e.g., about a 1:9 ratio of fine lactose to coarse lactose) to improve flow.

Tables 2 and 3 show non-limiting examples of grades/types of lactose that can be used in DPI formulations. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size <5 microns in the inspiration air. High FPF, and hence good DPI performance, can be obtained from, e.g., DPI formulations having an approximately 1:9 ratio of fine lactose (e.g., Lactohale® 300) to coarse lactose (e.g., Respitose® SV003) and about 20% w/w overages to avoid deposition of the drug in the capsule shell or the DPI device and to deliver essentially all of the drug to the airways.

TABLE 2

| | | Range of Particle Sizes (μm) | | |
|---|---|---|---|---|
| Product | Type | 10% | 50% | 90% |
| Lactohale ® | LH200 | <9 | <69 | <141 |
| InhaLac ® | 230 | <35 | <93 | <138 |
| Respitose ® | ML001 | <4 | <43 | <146 |
| | ML003 | <4 | <35 | <106 |
| | SV003 | <30 | <59 | <90 |
| | SV004 | <32 | <61 | <93 |

TABLE 3

| | | Range of Particle Sizes | | | |
|---|---|---|---|---|---|
| Product | Type | <45 μm | <100 μm | <150 μm | <250 μm |
| Respitose ® | ML003 | 65% | 98% | 100% | NA |
| Respitose ® | ML002 | 65% | 98% | NA | 100% |

Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol. Most DPIs are breath-activated (“passive”), relying on the user's inhalation for aerosol generation. Examples of passive DPIs include without limitation Airmax®, Novolizer® and Otsuka DPI (compact cake). The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose). A peptide product can also be administered by other routes, such as orally. An oral formulation can contain a peptide product and conventional excipients known in the art, and optionally an absorption enhancer such as sodium V-[8-(2-hydroxybenzoyl) aminocaprylate] (SNAC). SNAC protects against enzymatic degradation via local buffering action and enhances GI absorption. An oral dosage form (e.g., a tablet, capsule or pill) can optionally have an enteric coating to protect its content from the strong acids and proteolytic enzymes of the stomach. In some embodiments, a peptide product is delivered from a sustained-release composition. As used herein, the term “sustained-release composition” encompasses sustained-release, prolonged-release, extended-release, delayed-release, slow-release and controlled-release compositions, systems and devices. In some embodiments, a sustained-release composition delivers a peptide product over a period of at least about 1 week, 2 weeks, 3 weeks, 1 month, 2 months, 3 months or longer. In some embodiments, a sustained-release composition is formulated as nanoparticles or microparticles composed of a biodegradable polymer and incorporating a peptide product. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. In further embodiments, a sustained-release composition is in the form of a depot that is generated when a mixture of a peptide product and a polymer is injected into a subject intramuscularly or subcutaneously. In certain embodiments, the polymer is or comprises PEG, polylactic acid (PLA) or polyglycolic acid (PGA), or a copolymer thereof (e.g., PLGA or PLA-PEG).

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug but can contain an appropriate fraction thereof so that taking multiple unit dosage forms achieves the therapeutically effective dose. Examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; and a capsule, cartridge or blister pre-loaded in or manually loaded into an inhaler. Alternatively, a pharmaceutical composition can be presented as a kit in which the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally). A kit can contain all active and inactive ingredients in unit dosage form or the active ingredient and inactive ingredients in two or more separate containers, and can contain instructions for administering or using the pharmaceutical composition to treat a medical condition disclosed herein. A kit can further contain a device for delivering the composition, such as an injection pen or an inhaler. In some embodiments, a kit contains a peptide product or a pharmaceutically acceptable salt thereof, or a pharmaceutical composition comprising the same, and instructions for administering or using the peptide product or the composition to treat a medical condition disclosed herein, such as insulin resistance, diabetes, metabolic syndrome, cardiovascular disease, obesity (including “chronic obesity” meaning obesity lasting more than one year or resulting in an obesity-related condition such as but not limited to insulin resistance, diabetes, metabolic syndrome, and/or cardiovascular disease), or a condition associated therewith (e.g., NASH or NAFLD). In certain embodiments, the kit further contains a device for delivering the peptide product or the composition, such as an injection pen or an inhaler.

### Methods of Treatment

The disclosure further provides uses of the dual agonist peptide products described herein to prevent and/or treat conditions associated with GLP1R and/or GCGR, such as but not limited to insulin resistance, diabetes, obesity, metabolic syndrome and cardiovascular diseases, and conditions associated therewith, such as NASH and PCOS. In some embodiments, the dual agonist peptide products can be used to treat hyperglycemia, insulin resistance, hyperinsulinemia, prediabetes, diabetes (including types 1 and 2, gestational and juvenile diabetes), diabetic complications, diabetic neuropathy, diabetic nephropathy, diabetic retinopathy, hyperlipidemia, hypercholesterolemia, hypertriglyceridemia, elevated blood levels of free fatty acids, obesity, metabolic syndrome, syndrome X, cardiovascular diseases (including coronary artery disease), atherosclerosis, acute cardiovascular syndrome, ischemia (including myocardial ischemia and cerebral ischemia/stroke), ischemia-reperfusion injury (including myocardial and cerebral IRI), infarction (including myocardial and cerebral infarction), angina, heart failure (e.g., congestive heart failure), peripheral vascular disease, thrombosis (e.g., deep vein thrombosis), embolism (e.g., pulmonary embolism), systemic inflammation (e.g., one characterized by elevated C-reactive protein blood level), and hypertension. The dual agonist peptide products can achieve their therapeutic effects through various mechanisms, including stimulation of blood glucose-dependent insulin secretion, increase in insulin sensitivity, stimulation of fat burning and reduction of body weight. The dual agonist peptide products can also promote, e.g., pancreatic beta-cell protection, cardioprotection, and/or wound healing.

In certain embodiments, methods comprise treating obesity and/or one or more symptoms or complications thereof in a subject in need thereof with a dual agonist peptide product of this disclosure. In embodiments, symptoms and/or complications are fatty liver disease(s) (FLD), wherein the FLD is selected from NASH and NAFLD. In certain embodiments, symptoms and/or complications of obesity include type 2 diabetes. Non-alcoholic fatty liver disease (NAFLD) is a condition in which fat accumulates in the liver in people who drink little or no alcohol. Non-alcoholic steatohepatitis (NASH) is a type of NAFLD that involves inflammation and liver damage, along with fat in your liver. Symptoms of NASH are often not noticeable, but some common symptoms include fatigue and pain in the upper right abdomen. Thus, “treatment of obesity” means an activity that alleviates or ameliorates any of the primary phenomena or secondary symptoms associated with obesity or other conditions described herein.

Nonalcoholic fatty liver disease (NAFLD) has become the most common cause of chronic liver disease in the United States. NAFLD is associated with metabolic disorders, such as type 2 diabetes mellitus, hypertension, dyslipidernia, and obesity. The pathophysiological mechanisms underlying the development of NAFLD are mainly the alterations in glucose and lipid metabolism, insulin resistance (IR) and insulin secretion, explaining the close association between NAFLD and T2D. Moreover, both patients with NAFLD and T2D often share the comorbidities associated with the metabolic syndrome, namely fasting hyperglycaemia, hypertension, hypertriglyceridemia, low high-density lipoprotein-cholesterol and/or abdominal fat accumulation. It is well established that individuals with NAFLD are more insulin resistant than those without NAFLD, even if they are lean and without diabetes.⁷ Both longitudinal and cross-sectional studies have demonstrated that increased IR is the earliest detectable abnormality in both prediabetes and overt T2D. The pancreas responds to increased IR by secreting more insulin and the liver decreases insulin clearance in order to increase peripheral insulin concentrations and prevent the development of diabetes. In NAFLD, IR is present in muscle, liver and adipose tissue. As a consequence, hepatic glucose production and adipose tissue lipolysis are only in part suppressed by insulin, resulting in higher fasting glucose and free fatty acid (FFA) concentrations, increasing the risk off T2D in these patients. Thus, there is a need to treat obesity in a subject with NAFLD or NASH.

The prevalence of NAFLD is estimated to be between 20% and 30% in the USA, and in the upcoming decade, it is also expected to be the leading cause of liver transplantation NAFLD exists on a spectrum from simple steatosis to steatohepatitis (nonalcoholic steatohepatitis [NASH]), which is marked by lobular inflammation and ballooning. Across the spectrum of disease, fibrosis progression can lead to the development of cirrhosis, although fibrosis progression is typically more common and rapid with NASH as opposed to simple steatosis.

Provided herein are methods for treating symptoms and underlying conditions (e.g. pathogenesis) of obesity including NAFLD and NASH.

NAFLD can be diagnosed with the evidence of hepatic steatosis on imaging or histology and lack of secondary causes of hepatic fat accumulation (alcoholic steatosis, medications, or hereditary disorders). Nonalcoholic fatty liver (NAFL) is defined as the presence of ≥5% hepatic steatosis without evidence of hepatocellular injury in the form of hepatocyte ballooning. While the majority of patients tend to remain in the benign NAFL stage, some patients progress to NASH, which is characterized by the presence of ≥5% steatosis and inflammation with hepatocyte injury, with or without fibrosis. An established scoring system for the assessment of histology in NAFLD is the NAFLD activity score (NAS). The NAS is quantified using the following characteristics: steatosis (0-3), lobular inflammation (0-3), and hepatocyte ballooning (0-2), which are added together to arrive at a final score (0-8). In embodiments provided herein are methods for treating obesity in a subject with NASH. In certain embodiments, the methods provide improvement in the NAS score of the subject.

The peptide products of this disclosure are dual agonist GLP-1/glucagon peptide products, which work in part to help restore normoglycaemia as well as ameliorate the risk of CVD or chronic kidney disease. In certain embodiments, SEQ ID NO: 1 promotes satiety and weight loss through direct effects on the central nervous system, reverses abnormal insulin and glucagon secretion in T2D, and have other beneficial metabolic effects relevant to the pathophysiology of NAFLD. In vitro and in vivo studies have suggested that GLP-IRAs induce hepatic gene expression of pathways that improve autophagy/endoplasmic reticalum stress, macrophage recruitment, and enhance mitochondrial function and hepatocyte fatty acid oxidation, resulting in a decrease in steatosis and inflammation.

Histologic endpoints to assess response to therapeutic intervention can be defined as improvement in NAS, resolution of NASH, or improvement in liver fibrosis. For resolution of NASH, it is defined as complete resolution of hepatocyte ballooning with inflammation score of 0 or 1 and no worsening of fibrosis. Hepatocyte ballooning is an important parameter in NASH and has been shown to correlate with progressive disease and fibrosis. The fat accumulation in the ballooned hepatocyte causes oxidative injury, endoplasmic reticulum dysfunction, and abnormalities of cytoskeleton evident as Mallory-Denk body. In embodiments, biomarkers may also be used as markers for treatment efficacy. For hepatic steatosis assessment, controlled attenuation parameter as part of vibration-controlled transient elastography (FibroScan) and MRI proton density fat fraction (MRI-PDFF) and multiparametric MRI (LiverMultiScan) are being used. For liver inflammation and ballooning, liver enzymes ALT and aspartate aminotransferase (AST) can be used. LiverMultiScan has gained attention for inflammation assessment but requires confirmatory data. Transient elastography, LiverMultiScan, MRI, or MR elastography can be used for analysis of fibrosis. Biomarkers are also available for accurate hepatocyte fibrosis assessment, including Pro-C3, FIB-4, NAFLD fibrosis score, along with the enhanced liver fibrosis score are commonly investigated noninvasive tools for fibrosis assessment.

The peptide products described herein can be used to treat other conditions associated with insulin resistance or/and obesity. Other conditions associated with insulin resistance or/and obesity include without limitation arthritis (e.g., osteoarthritis), low back pain, breathing disorders (e.g., asthma, obesity hypoventilation syndrome [Pickwickian syndrome] and obstructive sleep apnea), dermatological disorders (e.g., diabetic ulcers, acanthosis nigricans, cellulitis, hirsutism, intertrigo and lymphedema), gastroenterological disorders (e.g., cholelithiasis [gallstone], gastroesophageal reflux disease [GERD] and gastroparesis), gout, hypercortisolism (e.g., Cushing's syndrome), kidney disorders (e.g., chronic kidney disease), liver disorders (e.g., fatty liver disease [FLD] including alcoholic and non-alcoholic FLD), neurological disorders (e.g., carpal tunnel syndrome, dementias [e.g., Alzheimer's disease and vascular dementia], meralgia paresthetica, migraines and multiple sclerosis), urological disorders (e.g., erectile dysfunction, hypogonadism and urinary incontinence), polycystic ovary syndrome, infertility, menstrual disorders, mood disorders (e.g., depression), and cancers (e.g., cancers of the endometrium, esophagus, colorectum, gallbladder, kidney, liver [e.g., hepatocellular carcinoma], pancreas and skin [e.g., melanoma], and leukemia). In certain embodiments, a dual agonist peptide product described herein is used to treat polycystic ovary syndrome (PCOS). In other embodiments, a peptide product is used to treat chronic kidney disease (CKD), also known as chronic kidney/renal failure (CKF/CRF). The most common causes of CKD are diabetes and long-term, uncontrolled hypertension. In further embodiments, a dual agonist peptide product described herein is used to treat fatty liver disease (FLD). In some embodiments, the FLD is non-alcoholic fatty liver disease (NAFLD), also understood to include metabolic fatty liver disease (MFLD). In certain embodiments, the NAFLD is non-alcoholic steatohepatitis (NASH). FLD, also known as hepatic steatosis, is characterized by excessive fat accumulation in the liver. FLD includes alcoholic fatty liver disease (AFLD) and NAFLD. Chronic alcoholism causes fatty liver due to production of toxic metabolites such as aldehydes during metabolism of alcohol in the liver. NAFLD is described below. FLD is associated with diabetes, obesity and metabolic syndrome. Fatty liver can develop into cirrhosis or a liver cancer (e.g., hepatocellular carcinoma [HCC]). Less than about 10% of people with cirrhotic AFLD develop HCC, but up to about 45% of people with NASH without cirrhosis may develop HCC. HCC is the most common type of primary liver cancer in adults and occurs in the setting of chronic liver inflammation. NAFLD is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., dyslipidemia and impaired glucose control). A liver can remain fatty without disturbing liver function, but a fatty liver can progress to become NASH, a condition in which steatosis is accompanied by inflammation, hepatocyte ballooning and cell injury with or without fibrosis of the liver. Fibrosis is the strongest predictor of mortality from NASH. NAFLD can be characterized by steatosis alone; steatosis with lobular or portal inflammation but without ballooning; steatosis with ballooning but without inflammation; or steatosis with inflammation and ballooning. NASH is the most extreme form of NAFLD. NASH is a progressive disease, with about 20% of patients developing cirrhosis of the liver and about 10% dying from a liver disease, such as cirrhosis or a liver cancer (e.g., HCC). NAFLD is the most common liver disorder in developed countries, and NASH is projected to supplant hepatitis C as the major cause of liver transplant in the U.S. by 2020. About 12-25% of people in the U.S. have NAFLD, with NASH affecting about 2-5% of people in the U.S. NAFLD, including NASH, is associated with insulin resistance, obesity and metabolic syndrome. For instance, insulin resistance contributes to progression of fatty liver to hepatic inflammation and fibrosis and thus NASH. Furthermore, obesity drives and exacerbates NASH, and weight loss can alleviate NASH. Therefore, the peptide products described herein, including GLP-1 receptor (GLP1R) agonists, glucagon receptor (GCGR) agonists and dual GLP1R/GCGR agonists, can be used to treat NAFLD, including NASH. In some embodiments, the dual agonist peptide products used to treat a condition associated with insulin resistance or/and obesity disclosed herein, such as fatty liver disease including NAFLD and NASH, is Pemvidutide, and/or derivatives thereof, and pharmaceutically acceptable salts thereof.

In some embodiments, the present dual agonist peptide(s) can be used to control blood glucose with reduction of one or more adverse events (i.e., an unexpected event that negatively impacts patient and/or animal welfare). Exemplary, non-limiting adverse events can include nausea, vomiting, diarrhea, abdominal pain and/or constipation. Adverse events may also include any known to those of ordinary skill in the art, such as those listed in industry resources and/or otherwise known to those of ordinary skill in the art (see, e.g., Medical Dictionary for Regulatory Activities (MedDRA) (Pharm., Med. Transl. Med. 2018) and/or Clark, M. J. Biomed. Inf., 54, April 2015, pp. 167-173). Such adverse events can be determined in humans using standard techniques as are typically used in clinical trials (e.g., doctor visit, surveys/questionnaires). As compared to the frequency and/or severity of such an adverse event that occurs upon administration of an agonist with unbalanced affinity for GLP-1R and GCGR (e.g., semaglutide) to a subject, the dual agonist peptides of this disclosure (e.g., any of SEQ ID NO. 1, or derivatives thereof) can decrease such frequency and/or severity thereof by, e.g., 5%, 10%, 20%, 40%, 50%, 60%, 70%, 80%, 90% of higher (up to 100%). In some embodiments, the dual agonist peptides of this disclosure (e.g., Pemvidutide) do not cause any adverse events.

A present dual agonist peptide product can be administered by any suitable route for treatment of a condition disclosed herein. Potential routes of administration of a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository)). In some embodiments, a peptide product is administered parenterally, such as subcutaneously, intravenously or intramuscularly. In other embodiments, a peptide product is administered by oral inhalation or nasal inhalation or insufflation. The therapeutically effective amount and the frequency of administration of, and the length of treatment with, a peptide product to treat a condition disclosed herein may depend on various factors, including the nature and severity of the condition, the potency of the compound, the route of administration, the age, body weight, general health, gender and diet of the subject, and the response of the subject to the treatment, and can be determined by the treating physician. In some embodiments, a peptide product is administered parenterally (e.g., subcutaneously (sc), intravenously (iv) or intramuscularly (im)) in a dose from about 0.01 mg to about 0.1, 1, 5 or 10 mg, or about 0.1-1 mg or 1-10 mg, over a period of about one week for treatment of a condition disclosed herein (e.g., one associated with insulin resistance or/and obesity, such as NASH or NAFLD). In further embodiments, a peptide product is administered parenterally (e.g., sc, iv or im) in a dose of about 0.1-0.5 mg, 0.5-1 mg, 1-5 mg or 5-10 mg over a period of about one week. In certain embodiments, a peptide product is administered parenterally (e.g., subcutaneously (SC), intravenous (IV) or intramuscular (IM)) in a dose of about 0.1-1 mg, or about 0.1-0.5 mg or 0.5-1 mg, over a period of about one week. One of skill in the art understands that an effective dose in a mouse, or other pre-clinical animal model, may be scaled for a human. In that way, through allometric scaling (also referred to as biological scaling) a dose in a larger animal may be extrapolated from a dose in a mouse to obtain an equivalent dose based on body weight or body surface area of the animal.

A peptide product can be administered in any suitable frequency for treatment of a condition disclosed herein (e.g., one associated with insulin resistance or/and obesity, such as NASH or NAFLD). In some embodiments, a dual agonist peptide product is administered, e.g., sc or iv once a day, once every two days, once every three days, twice a week, once a week or once every two weeks. In certain embodiments, a peptide product is administered, e.g., SC, IV, or IM once a week. A dual agonist peptide product can be administered at any time of day convenient to the patient. A dual agonist peptide product can be taken substantially with food (e.g., with a meal or within about 1 hour or 30 minutes before or after a meal) or substantially without food (e.g., at least about 1 or 2 hours before or after a meal). The length of treatment of a medical condition with a dual agonist peptide product can be based on, e.g., the nature and severity of the condition and the response of the subject to the treatment, and can be determined by the treating physician. In some embodiments, a dual agonist peptide product is administered chronically to treat a condition disclosed herein, such as at least about 2 months, 3 months, 6 months, 1 year, 1.5 years, 2 years, 3 years, 5 years, 10 years or longer. A dual agonist peptide product can also be taken pro re nata (as needed) until clinical manifestations of the condition disappear or clinical targets are achieved, such as blood glucose level, blood pressure, blood levels of lipids, body weight or body mass index, waist-to-hip ratio or percent body fat, or any combination thereof. If clinical manifestations of the condition re-appear or the clinical targets are not maintained, administration of the dual agonist peptide product can resume. The disclosure provides a method of treating a medical condition described herein, comprising administering to a subject in need of treatment a therapeutically effective amount of a peptide product described herein or a pharmaceutically acceptable salt thereof, or a pharmaceutical composition comprising the same. The disclosure further provides a peptide product described herein or a pharmaceutically acceptable salt thereof, or a composition comprising the same, for use as a medicament. In addition, the disclosure provides for the use of a peptide product described herein or a pharmaceutically acceptable salt thereof in the preparation of a medicament. The medicament containing the peptide product can be used to treat any medical condition described herein. The peptide product can optionally be used in combination with one or more additional therapeutic agents.

A dual agonist peptide product described herein can be administered as the sole active agent, or optionally be used in combination with one or more other dual agonist peptide products, and/or additional therapeutic agents to treat any disorder disclosed herein, such as insulin resistance, diabetes, obesity, metabolic syndrome or a cardiovascular disease, or any condition associated therewith, such as NASH or NAFLD. In some embodiments, the one or more additional therapeutic agents are selected from antidiabetic agents, anti-obesity agents (including lipid-lowering agents and pro-satiety agents), anti-atherosclerotic agents, anti-inflammatory agents, antioxidants, antifibrotic agents, anti-hypertensive agents, and combinations thereof. Antidiabetic agents include without limitation: AMP-activated protein kinase (AMPK) agonists, including biguanides (e g., buformin and metformin); peroxisome proliferator-activated receptor gamma (PPAR-γ) agonists, including thiazolidinediones (e.g., balaglitazone, ciglitazone, darglitazone, englitazone, lobeglitazone, netoglitazone, pioglitazone, rivoglitazone, rosiglitazone and troglitazone), MSDC-0602K and saroglitazar (dual PPAR-α/γ agonist); glucagon-like peptide-1 (GLP-1) receptor agonists, including exendin-4, albiglutide, dulaglutide, exenatide, liraglutide, lixisenatide, semaglutide, taspoglutide, CNT0736, CNT03649, HM11260C (LAPS-Exendin), NN9926 (OG9S7GT), TT401 and ZYOGl; dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, teneligliptin, trelagliptin and vildagliptin; sodium-glucose transport protein 2 (SGLT2) inhibitors, including canagliflozin (also inhibits SGLT1), dapagliflozin, empagliflozin, ertugliflozin, ipragliflozin, luseogliflozin, remogliflozin etabonate, sotagliflozin (also inhibits SGLT1) and tofogliflozin; blockers of ATP-dependent K+(KA_{TP}) channels on pancreatic beta cells, including rneglitinides (e.g., mitiglinide, nateglinide and repagiinide) and sulfonylureas {including first generation (e.g., acetohexamide, carbutamide, chlorpropamide, giycyclamide [tolhexamide], metahexamide, tolazamide and tolbutamide) and second generation (e.g., glibenclamide, glyburide, glibornuride, gliclazide, glimepiride, glipizide, gliquidone, glisoxepide and glyclopyramide); insulin and analogs thereof, including fast-acting insulin (e.g., insulin aspari insulin glulisine and insulin lispro), intermediate-acting insulin (e.g., NPH insulin), and long-acting insulin (e.g., insulin degludec, insulin detemir and insulin glargine); and/or, analogs, derivatives and salts thereof. In certain embodiments, the antidiabetic agent is or includes a biguanide (e.g., metformin), a thiazolidinedione (e.g., pioglitazone or rosiglitazone) or a SGLT2 inhibitor (e.g., empagliflozin or tofogliflozin), or any combination thereof. Anti-obesity agents include, but are not limited to: appetite suppressants (anorectics), including amphetamine, dexamphetamine, amfepramone, clobenzorex, mazindol, phentermine (with or without topiramate) and lorcaserin; pro-satiety agents, including ciliary neurotrophic factor (e.g., axokine) and longer-acting analogs of amylin, calcitonin, cholecystokinin (CCK), GLP-1, leptin, oxyntomodulin, pancreatic polypeptide (PP), peptide YY (PYY) and neuropeptide Y (NPY); lipase inhibitors, including caulerpenyne, cetilistat, ebelactone A and B, esterastin, lipstatin, orlistat, percyquinin, panclicin A-E, valilactone and vibralactone; antihyperlipidemic agents; and analogs, derivatives and salts thereof. Antihyperlipidemic agents include without limitation: HMG-CoA reductase inhibitors, including statins {e.g., atorvastatin, cerivastatin, fluvastatin, mevastatin, monacolins (e.g., monacolin K (lovastatin), pitavastatin, pravastatin, rosuvastatin and simvastatin} and flavanones (e.g., naringenin); squalene synthase inhibitors, including lapaquistat, zaragozic acid and RPR-107393; acetyl-CoA carboxylase (ACC) inhibitors, including anthocyanins, avenaciolides, chloroacetylated biotin, cyclodim, diclofop, haloxyfop, soraphens (e.g., soraphen A₁ₐ), 5-(tetradecyloxy)-2-furancarboxylic acid (TOFA), CP-640186, GS-0976, NDI-010976; 7-(4-propyloxy-phenylethynyl)-3,3-dimethyl-3,4dihydro-2H-benzo[b][1,4]dioxepine; N-ethyl-N′-(3-{[4-(3,3-dimethyl-1-oxo-2-oxa-7-azaspiro[4.5]dec-7-yl)piperidin-1-yl]-carbonyl}-1-benzothien-2-yl)urea; 5-(3-acetamidobut-1-ynyl)-2-(4-propyloxyphenoxy)thiazole; and 1-(3-{[4-(3,3-dimethyl-1-oxo-2-oxa-7-azaspiro[4.5]dec-7-yl)piperidin-1-yl]-carbonyl}-5-(pyridin-2-yl)-2-thienyl)-3-ethylurea; PPAR-α agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid); PPAR-δ agonists, including elafibranor (dual PPAR-α/γ agonist), GFT505 (dual PPAR-α/γ agonist), GW0742, GW501516 (dual PPAR-β/δ agonist), sodelglitazar (GW677954), MBX-8025, and isoflavones (e.g., daidzein and genistein); PPAR-γ agonists, including thiazolidinediones {supra), saroglitazar (dual PPAR-α/γ agonist), 4-oxo-2-thioxothiazolines (e.g., rhodanine), berberine, honokiol, perfluorononanoic acid, cyclopentenone prostaglandins (e.g., cyclopentenone 15-deoxy-A-prostaglandin J₂ [15d-PGJ₂]), and isoflavones (e.g., daidzein and genistein); liver X receptor (LXR) agonists, including endogenous ligands (e.g., oxysterols such as 22(i?)-hydroxycholesterol, 24(A)-hydroxy cholesterol, 27-hydroxycholesterol and cholestenoic acid) and synthetic agonists (e.g., acetyl-podocarpic dimer, hypocholamide, A(X-di methyl-3 b-hydroxy-cholenamide [DMHCA], GW3965 and T0901317); retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346); inhibitors of acyl-CoA cholesterol acyltransferase (ACAT, aka sterol G-acyl transferase [SOAT], including ACAT1 [SOAT1] and ACAT2 [SOAT2]), including avasimibe, pactimibe, pellitorine, terpendole C and flavanones (e.g., naringenin); inhibitors of stearoyl-CoA desaturase-1 (SCD-1, aka stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl)thiazol-2-yl]benzamide and 4-ethylamino-3-(2-hydroxyethoxy)-N-[5-(3-trifluoromethylbenzyl)thiazol-2-yl]benzamide; 1′-{6-[5-(pyridin-3-ylmethyl)-1,3,4-oxadiazol-2-yl]pyridazin-3-yl}-5-(trifluoromethyl)-3,4-dihydrospiro[chromene-2,4′-piperidine]; 5-fluoro-1-{6-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H,3H-spiro[1,5-benzoxazepine-2,4′-piperidin]-1′-yl]-N-(2-hydroxy-2-pyridin-3-ylethyl)pyridazine-3-carboxamide; 6-[4-(2-methylbenzoyl)piperidin-1-yl]pyridazine-3-carboxylic acid (2-hydroxy-2-pyridin-3-ylethyl)amide; 4-(2-chlorophenoxy)-N-[3-(methyl carbamoyl)phenyl]piperidine-1-carboxamide; the cis-9,trans-11 isomer and the trans-10,cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs; cholesterylester transfer protein (CETP) inhibitors, including anacetrapib, dalcetrapib, evacetrapib, torcetrapib and AMG 899 (TA-8995); inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP-targeting microRNAs (e.g., miRNA-30c), and MTTP-targeting siRNAs; GLP-1 receptor agonists; fibroblast growth factor 21 (FGF21) and analogs and derivatives thereof, including BMS-986036 (pegylated FGF21); inhibitors of pro-protein eonvertase subtilisin/kexin type 9 (PCSK9) activity or expression, including berberine (reduces PC8K9 level), annexin A2 (inhibits PCSK9 activity), anti-PCSK9 antibodies (e.g., alirocumab, bococizumab, evolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g, inclisiran [ALN-PCS] and ALN-PCSO2); apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CH1, 5A-CH2, 5A-H1, 18 A, 37 pA [18A-P-18A], ELK, ELK-1A, ELK-1F, ELK-1K1AlE, ELK-1L1K, ELK-1W, ELK-2A, ELK-2A2K2E, ELK-2E2K, ELK-2F, ELK-3 E3EK, ELK-3E3K3A, ELK-3E3LK, ELK-PA, ELK-P2A, ELKA, ELKA-CH2, ATI-5261, CS-6253, ETC-642, FAMP, FREL and KRES and apoE mimetics (e.g., Ac-hE18A-NH₂, AEM-28, Ac-[R]hEl 8 A-NH₂, AEM-28-14, EpK, hEp, mR18L, COG-112, COG-133 and COG-1410); omega-3 fatty acids, including docosahexaenoic acid (DHA), docosapentaenoic acid (DPA), eicosapentaenoic acid (EPA), a-linolenic acid (ALA), fish oils (which contain, e.g., DHA and EPA), and esters (e.g., glyceryl and ethyl esters) thereof; and analogs, derivatives and salts thereof. In certain embodiments, the anti-obesity agent is or includes a lipase inhibitor (e.g., orlistat) or/and an antihyperlipidemic agent (e.g., a statin such as atorvastatin, or/and a fibrate such as fenofibrate). Antihypertensive agents include without limitation: antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (ATIIl) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone); diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothi azide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine; calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyri dines (e.g., diltiazem and verapamil); (α₂-adrenoreceptor agonists, including clonidine, guanabenz, guanfacine, methyldopa and moxonidine; cal-adrenoreceptor antagonists (alpha blockers), including doxazosin, indoramin, nicergoline, phenoxybenzamine, phentolamine, prazosin, terazosin and tolazoline; β-adrenoreceptor (β1 or/and β₂) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol; mixed alpha/beta blockers, including bucindolol, carvedilol and labetalol; endothelin receptor antagonists, including selective ETA receptor antagonists (e.g., ambrisentan, atrasentan, edonentan, sitaxentan, zibotentan and BQ-123) and dual ET_{A}/ET_{B} antagonists (e.g., bosentan, macitentan and tezosentan); other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin Ei (alprostadil) and analogs thereof (e.g., limaprost amd misoprostol), prostacyclin and analogs thereof (e.g., ataprost, beraprost [e.g., esuberaprost], 5,6,7-trinor-4,8-inter-w-phenylene-9-fluoro-PG12, carbacyclin, isocarbacyclin, clinprost, ciprostene, eptaloprost, cicaprost, iloprost, pimilprost, SM-10906 (des-methyl pimilprost), naxaprostene, taprostene, treprostinil, CS-570, OP-2507 and TY-11223), non prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, and protein kinase C (PKC) inhibitors (e.g., BIM-1, BIM-2, BIM-3, BIM-8, chelerythrine, cicletanine, gossypol, miyabenol C, myricitrin, ruboxistaurin and verbascoside; minerals, including magnesium and magnesium sulfate; and analogs, derivatives and salts thereof. In certain embodiments, the antihypertensive agent is or includes a thiazide or thiazide like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof. In some embodiments, a peptide product described herein is used in combination with one or more additional therapeutic agents to treat NAFLD, such as NASH. In some embodiments, the one or more additional therapeutic agents are selected from antidiabetic agents, anti-obesity agents, anti-inflammatory agents, antifibrotic agents, antioxidants, anti-hypertensive agents, and combinations thereof. Therapeutic agents that can be used to treat NAFLD (e.g., NASH) include without limitation: PPAR agonists, including PPAR-δ agonists (e.g., MBX-8025, elafibranor [dual PPAR-α/δ agonist] and GW501516 [dual PPAR-β/δ agonist]) and PPAR-γ agonists (e.g., thiazolidinediones such as pioglitazone, and saroglitazar [dual PPAR-α/γ agonist])-PPAR-δ and -γ agonism increases insulin sensitivity, PPAR-α agonism reduces liver steatosis and PPAR-δ agonism inhibits activation of macrophages and Kupffer cells; farnesoid X receptor (FXR) agonists, such as obeticholic acid and nonsteroidal FXR agonists like GS-9674 reduce liver gluconeogenesis, lipogenesis, steatosis and fibrosis; fibroblast growth factor 19 (FGF19) and analogs and derivatives thereof, such as NGM-282-FGF19 analogs reduce liver gluconeogenesis and steatosis; fibroblast growth factor 21 (FGF21) and analogs and derivatives thereof, such as BMS-986036 (pegylated FGF21)-FGF21 analogs reduce liver steatosis, cell injury and fibrosis; HMG-CoA reductase inhibitors, including statins (e.g., rosuvastatin)—statins reduce steatohepatitis and fibrosis; ACC inhibitors, such as NDI-010976 (liver-targeted) and GS-0976-ACC inhibitors reduce de novo lipogenesis and liver steatosis; SCD-1 inhibitors, such as aramchol-SCD-1 inhibitors reduce liver steatosis and increase insulin sensitivity; SGLT2 inhibitors, such as canagliflozin, ipragliflozin and luseogliflozin-SGLT2 inhibitors reduce body weight, liver ALT level and fibrosis; antagonists of CCR2 or/and CCR5, such as cenicriviroc-antagonists of CCR2 (binds to CCL2 [MCP1]) and CCR5 (binds to CCL5 [RANTES]) inhibit activation and migration of inflammatory cells (e.g., macrophages) to the liver and reduce liver fibrosis; apoptosis inhibitors, including apoptosis signal-regulating kinase 1 (ASK1) inhibitors (e.g., selonsertib) and caspase inhibitors (e.g., emricasan [pan-caspase inhibitor])-apoptosis inhibitors reduce liver steatosis and fibrosis; lysyl oxidase-like 2 (LOXL2) inhibitors, such as simtuzumab-LOXL2 is a key matrix enzyme in collagen formation and is highly expressed in the liver; galectin-3 inhibitors, such as GR-MD-02 and TD139-galectin-3 is critical for development of liver fibrosis; antioxidants, including vitamin E (e.g., a-tocopherol) and scavengers of reactive oxygen species (ROS) and free radicals (e.g., cysteamine, glutathione, melatonin and pentoxifylline [also anti-inflammatory via inhibition of TNF-α and phosphodiesterases])-vitamin E reduces liver steatosis, hepatocyte ballooning and lobular inflammation; and, analogs, derivatives and salts thereof. In some embodiments, a peptide product described herein is used in conjunction with a PPAR agonist (e.g., a PPAR-8 agonist such as elafibranor or/and a PPAR-γ agonist such as pioglitazone), a HMG-CoA reductase inhibitor (e.g., a statin such as rosuvastatin), an FXR agonist (e.g., obeticholic acid) or an antioxidant (e.g., vitamin E), or any combination thereof, to treat NAFLD (e.g., NASH). In certain embodiments, the one or more additional therapeutic agents for treatment of NAFLD (e.g., NASH) are or include vitamin E or/and pioglitazone. Other combinations may also be used as would be understood by those of ordinary skill in the art.

Pharmacokinetic (“PK”) parameters can be estimated using Phoenix® WinNonlin® version 8.1 or higher (Certara USA, Inc., Princeton, New Jersey). A non-compartmental approach consistent with the extravascular route of administration can be used for parameter estimation. The individual plasma concentration-time data can be used for pharmacokinetic calculations. In addition to parameter estimates for individual animals, descriptive statistics (e.g. mean, standard deviation, coefficient of variation, median, min, max) can be determined, as appropriate. Concentration values that are below the limit of quantitation can be treated as zero for determination of descriptive statistics and pharmacokinetic analysis. Embedded concentration values that are below the limit of quantitation can be excluded from pharmacokinetic analysis. All parameters can be generated from individual dual agonist peptide (or derivatives and/or metabolites thereof) concentrations in plasma from test article-treated groups on the day of dosing (Day 1). Parameters can be estimated using nominal dose levels, unless out of specification dose formulation analysis results are obtained, in which case actual dose levels can be used. Parameters can be estimated using nominal sampling times; if bioanalytical sample collection deviations are documented, actual sampling times can be used at the affected time points. Bioanalytical data can be used as received for the pharmacokinetic analysis and can be presented in tables and figures in the units provided. Pharmacokinetic parameters can be calculated and presented in the units provided by the analytical laboratory (the order of magnitude can be adjusted appropriately for presentation in the report, e.g. h*ng/mL converted to h*μg/mL). Descriptive statistics (e.g., mean, standard deviation, coefficient of variation, median, min, max) and pharmacokinetic parameters can be determined to three significant figures, as appropriate. Additional data handling items can be documented as needed. PK parameters to be determined, as data permit, can include but are not limited to the following: Cₘₐₓ: Maximum observed concentration; DN Cₘₐₓ: dose normalized maximum concentration, calculated as Cₘₐₓ/dose; Tₘₐₓ: time of maximum observed concentration; AUC₀₋ₜ: area under the curve from time 0 to the time of the last measurable concentration, calculated using the linear trapezoidal rule; AUC₀₋₉₆: area under the curve from time 0 to hour 96, calculated using the linear trapezoidal rule; DN AUC₀₋₉₆: dose normalized AUC₀₋₉₆, calculated as AUC₀₋₉₆/dose; AUC_{0-inf}: area under the curve from time 0 to infinity (Day 1 only), calculated as AUC_{0-inf}=AUC₀₋ₜ+Ct/λ_{z}, where Cₜ is the last observed quantifiable concentration and az is the elimination rate constant; t_{1/2}: elimination half-life, calculated as ln(2)/λ_{z}. Additional parameters and comparisons (e.g., sex ratios, dose proportionality ratios, etc.) can also be determined, as would be understood by those of ordinary skill in the art.

In some embodiments, this disclosure provides pharmaceutical dosage formulation(s) comprising Pemvidutide wherein the peptide product is modified with a hydrophobic surfactant; the dosage is configured to induce weight loss, with reduction of one or more adverse events wherein the subject has fatty liver disease and may also suffer from type 2 diabetes, wherein the adverse events being selected from nausea, vomiting, diarrhea, abdominal pain and constipation, upon administration to a mammal.

“Reducing,” or “reduction of” adverse effects or events refers to a reduction in the degree, duration, and/or frequency of adverse effects experienced by a subject and incidence in a group of subjects following administration of an agonist with about balanced affinity to GLP1R and GCGR. Such reduction encompasses the prevention of some adverse effects that a subject would otherwise experience in response to an agonist with unbalanced affinity to GLP1R and GCGR. Such reduction also encompasses the elimination of adverse effects previously experienced by a subject following administration of an agonist with unbalanced affinity to GLP1R and GCGR. In some embodiments, “reducing,” or “reduction of” adverse effects encompass a reduction of gastrointestinal side effects wherein the adverse events are reduced to zero or undetectable levels. In other embodiments, adverse effect is reduced to level equivalent to untreated subjects but not completely eliminated. Moreover, administration of analogs with unbalanced affinity toward GLP-1R or GCGR to a mammal may lead to the need for an excessively high dose to maximally activate the receptor with weaker sensitivity toward the ligand, thus leading to a potential for exceeding the biologically effective dose level for the other ligand and causing dose-related, undesired side effects.

In preferred embodiments, this disclosure provides methods of reducing body weight in a human being with fatty liver disease, wherein the method comprises administering Pemvidutide once weekly in an amount from at least 1.8 mg up to 2.4 mg to the human being in need thereof; and, wherein the human being may suffer from type 2 diabetes and wherein the fatty liver disease is non-alcoholic fatty liver disease (NAFLD) or non-alcoholic steatohepatitis (NASH). In some preferred embodiments of such methods, the body weight of the human being is reduced by at least 3% from baseline at week 12. In some preferred embodiments of such methods, the body weight of the human being is reduced by at least 4% from baseline at week 12. In some preferred embodiments of such methods, the Pemvidutide is administered once weekly in an amount of 1.8 mg. In some preferred embodiments of such methods, the Pemvidutide is administered once weekly in an amount of 2.4 mg.

In some preferred embodiments, this disclosure provides methods of reducing liver fat content as determined by MRI-PDFF in a human being with fatty liver disease, the method comprising administering Pemvidutide once weekly for at least 12 continuous weeks in an amount from at least about 1.2 mg up to about 2.4 mg to the human being, wherein the human being: has been diagnosed with fatty liver disease that is non-alcoholic fatty liver disease (NAFLD) or non-alcoholic steatohepatitis (NASH); has a body mass index (BMI kg/m²) of greater than about 27; and, has a liver fat content of at least about 10% as measured by MRI-PDFF. In some preferred embodiments of such methods, a dose of about 1.2 mg per week induces in a population of human beings an absolute reduction in liver fat content of at least about 7%, optionally at least about 9%, that is significant (p<0.001) as compared to placebo at week 12. In some preferred embodiments of such methods, a dose of about 1.8 mg per week induces in a population of human beings an absolute reduction in liver fat content of at least about 12.5%, optionally at least about 14%, that is significant (p<0.001) as compared to placebo at week 12.

In some preferred embodiments of such methods, a dose of about 2.4 mg per week induces in a population of human beings an absolute reduction in liver fat content of at least about 10%, optionally at least about 11%, that is significant (p<0.001) as compared to placebo at week 12. In some preferred embodiments of such methods, a dose of about 1.2 mg per week induces in a population of human beings a relative reduction in liver fat content of at least about 40%, optionally at least about 45%, that is significant (p<0.001) as compared to placebo at week 12. In some preferred embodiments of such methods, a dose of about 1.8 mg per week induces in a population of human beings a relative reduction in liver fat content of at least about 60%, optionally at least about 65%, that is significant (p<0.001) as compared to placebo at week 12. In some preferred embodiments of such methods, a dose of about 2.4 mg per week induces in a population of human beings a relative reduction in liver fat content of at least about 50%, optionally at least about 55%, that is significant (p<0.001) as compared to placebo at week 12. In some preferred embodiments of such methods, a dose of about 1.2 mg per week induces in at least about 55%, optionally at least about 60%, of a population of human beings at least about a 30% reduction in liver fat content that is significant (p<0.001) as compared to placebo at week 12. In some preferred embodiments of such methods, a dose of about 1.8 mg per week induces in at least about 80%, optionally at least about 85%, of a population of human beings at least about a 30% reduction in liver fat content that is significant (p<0.001) as compared to placebo at week 12. In some preferred embodiments of such methods, a dose of about 2.4 mg per week induces in at least about 75%, optionally at least about 80%, of a population of human beings at least about a 30% reduction in liver fat. In some preferred embodiments of such methods, a dose of about 1.2 mg per week induces in at least about 35%, optionally at least about 38%, of a population of human beings at least about a 50% reduction in liver fat content that is significant (p<0.001) as compared to placebo at week 12. In some preferred embodiments of such methods, a dose of about 1.8 mg per week induces in at least about 55%, optionally at least about 60%, of a population of human beings about a 50% reduction in liver fat content that is significant (p<0.001) as compared to placebo at week 12. In some preferred embodiments of such methods, a dose of about 2.4 mg per week induces in at least about 60%, optionally at least about 65%, of a population of human beings at least about a 50% reduction in liver fat content that is significant (p<0.001) as compared to placebo at week 12. In some preferred embodiments of such methods, a dose of about 1.2 mg per week induces liver fat content normalization to less than or equal to about 5% liver fat content in at least about 20% of a population of human beings that is significant (p<0.05) as compared to placebo at week 12. In some preferred embodiments of such methods, a dose of about 1.8 mg per week induces liver fat content normalization to less than or equal to about 5% liver fat content in at least about 50%, optionally at least about 55%, of a population of human beings that is significant (p<0.0001) as compared to placebo at week 12. In some preferred embodiments of such methods, a dose of about 2.4 mg per week induces liver fat content normalization to less than or equal to about 5% liver fat content in at least about 50% of a population of human beings that is significant (p<0.001) as compared to placebo at week 12.

In some preferred embodiments, this disclosure provides methods of inducing weight loss in a human being with fatty liver disease, the method comprising administering Pemvidutide once weekly for at least 12 continuous weeks in an amount from at least about 1.2 mg up to about 2.4 mg to the human being, wherein the human being: has been diagnosed with fatty liver disease that is non-alcoholic fatty liver disease (NAFLD) or non-alcoholic steatohepatitis (NASH); has a body mass index (BMI kg/m²) of greater than about 27; and, has a liver fat content of at least about 10% as measured by MRI-PDFF. In some preferred embodiments of such methods, the human being is not diabetic and a dose of about 1.2 mg per week reduces the body weight of the human being by at least about 2.5%, optionally at least about 3%, that is significant (p<0.001) as compared to placebo at week 12. In some preferred embodiments of such methods, the human being is not diabetic and a dose of about 1.8 mg per week reduces the body weight of the human being by at least about 4%, optionally about 5%, that is significant (p<0.001) as compared to placebo at week 12. In some preferred embodiments of such methods, the human being is not diabetic a dose of about 2.4 mg per week reduces the body weight of the human being by at least about 2.5%, optionally about 3.5%, that is significant (p<0.001) as compared to placebo at week 12. In some preferred embodiments of such methods, the human being has type 2 diabetes and a dose of about 1.2 mg per week reduces the body weight of the human being by at least about 2%, optionally at least about 3%, at week 12. In some preferred embodiments of such methods, the human being has type 2 diabetes and a dose of about 1.8 mg per week reduces the body weight of the human being by about 2.5%, optionally at least about 3.5%, that is significant (p<0.05) as compared to placebo at week 12. In some preferred embodiments of such methods, the human being has type 2 diabetes and a dose of about 2.4 mg per week reduces the body weight of the human being by about 3%, optionally at least about 4%, that is significant (p<0.05) as compared to placebo at week 12. In some preferred embodiments of such methods, a dose of about 1.2 mg per week reduces the body weight of a population of human beings by at least about 3% that is significant (p<0.001) as compared to placebo at week 12. In some preferred embodiments of such methods, a dose of about 1.8 mg per week reduces the body weight of a population of human beings by at least about 4% that is significant (p<0.001) as compared to placebo at week 12. In some preferred embodiments of such methods, a dose of about 2.4 mg per week reduces the body weight of a population of human beings by at least about 3%, optionally at least about 3.5%, that is significant (p<0.001) as compared to placebo at week 12.

In some preferred embodiments of the methods herein, a dose of about 1.2 mg per week induces ALT reduction of at least about 11% in a population of human beings at week 12. In some preferred embodiments of the methods herein, a dose of about 1.8 mg per week induces ALT reduction of at least about 13% in a population of human beings that is significant (p<0.05) as compared to placebo at week 12. In some preferred embodiments of the methods herein, a dose of about 2.4 mg per week induces ALT reduction of at least about 13% in a population of human beings that is significant (p<0.05) as compared to placebo at week 12. In some preferred embodiments of the methods herein, a dose of about 1.2 mg per week induces ALT reduction of at least about 19% at week 12 in a population of human beings with a baseline ALT of greater than or equal to about 30 IU/L. In some preferred embodiments of the methods herein, a dose of about 1.8 mg per week induces ALT reduction of at least about 20% at week 12 in a population of human beings with a baseline ALT of greater than or equal to about 30 IU/L. In some preferred embodiments of the methods herein, a dose of about 2.4 mg per week induces ALT reduction of at least about 20%, optionally at least about 25%, at week 12 that is significant (p<0.005) as compared to placebo in a population of human beings with a baseline ALT of greater than or equal to about 30 IU/L.

In some preferred embodiments, the Pemvidutide is administered by parenteral injection. In some preferred embodiments, the Pemvidutide is administered by subcutaneous injection. In some preferred embodiments, the human being has a body mass index (BMI kg/m²) of at least 27. In some preferred embodiments, the human being has a body mass index (BMI kg/m²) of 30 or greater. In some preferred embodiments, the human being has level of liver fat as measured by MRI-PDFF of 10% or greater before treatment. In some preferred embodiments, the absolute reduction in liver fat as determined using MRI-PDFF is any of about 8%, 10%, 12% or preferably about 15% after 12 weeks treatment. In some preferred embodiments, the relative reduction in liver fat as determined using MRI-PDFF to baseline is greater than about any of 40%, 50% or 60% after 12 weeks treatment. In some preferred embodiments, a steady state dose is achieved after a dose escalating phase having a duration of two to four weeks, or any of about six, eight, ten, 12, or 16 weeks. In some preferred embodiments, the Pemvidutide is administered from a liquid comprising at least about 2.5 mg/ml Pemvidutide. In some embodiments, the Pemvidutide is administered from a pharmaceutical dosage form as an aqueous formulation comprising one or more of polysorbate 20, Arginine, or Mannitol.

In some embodiments, this disclosure provides methods for reducing body weight in a human being with fatty liver disease, the method comprising administering pemvidutide once weekly in an amount from at least about 1.2 to about 2.4 mg (preferably about 1.2 mg, about 1.8 mg, or about 2.4 mg) to the human being in need thereof for at least about 24 weeks; wherein the human being optionally has type 2 diabetes and/or optionally wherein the fatty liver disease is non-alcoholic fatty liver disease (NAFLD) or non-alcoholic steatohepatitis (NASH). In some embodiments, the pemvidutide is administered once weekly for 24 weeks in an amount of 1.2 mg. In some such embodiments, this disclosure provides methods for administering pemvidutide once weekly at a dose of about 1.2 mg, 1.8 mg, or 2.4 mg, wherein the relative reduction in liver fat as measured by MRI-PDFF compared to baseline is about 30% to about 50% after 24 weeks of the once weekly dosing of pemvidutide, wherein the relative reduction is statistically significant defined as p<0.001 or p<0.0001. In some embodiments, the 1.8 mg and 2.4 mg once weekly doses of pemvidutide induce at least 40% reduction in liver fat after 24 weeks wherein the reduction is significant defined as p<0.001 or p<0.01 as compared to placebo. In some embodiments, defatting of the liver as measured by MRI-PDFF compared to baseline is about 30% after 24 weeks of once weekly dosing of about 1.8 mg pevidutide as compared to baseline in the human being. In some embodiments, liver volume as measured by MRI-PDFF compared to baseline is reduced after 24 weeks of once weekly dosing of about 1.2 mg, about 1.8, or about 2.4 mg pevidutide as compared to placebo. In some embodiments, alanine aminotransferase (ALT) is reduced after 24 weeks of once weekly dosing of about 1.2 mg, about 1.8 mg, or about 2.4 mg as compared to placebo, optionally wherein the human being has baseline ALT of >30 IU/L before dosing. In some embodiments, the reduction in iron-corrected T1 is superior to 80 ms in about 80% of subjects, optionally wherein the reduction is significant defined as p<0.05 or p<0.005 following 24 weeks of once weekly dosing of about 1.2 mg, about 1.8, or about 2.4 mg pemvidutide. In some embodiments, once weekly dosing of about 1.2 mg, about 1.8 mg, or about 2.4 mg of pemvidutide per week for 24 weeks results in weight loss in non-diabetic and/or diabetic patients as compared to placebo. In some embodiments, once weekly dosing of about 1.2 mg, about 1.8 mg, or about 2.4 mg of pemvidutide per week for 24 weeks results in lower serum lipid levels as compared to placebo. In some embodiments, once weekly dosing of about 1.2 mg, about 1.8 mg, or about 2.4 mg pemvidutide per week for 24 weeks reduced blood pressure without significantly increasing heart rate, optionally wherein systolic blood pressure is significantly reduced as defined as p<0.05) by the 2.4 mg weekly dose. In some embodiments once weekly dosing of about 1.2 mg, about 1.8 mg, or about 2.4 mg per week of pemvidutide for 24 weeks improves glycemic parameters, optionally wherein the glycemic parameters are a reduction of fasting glucose and/or HbAlc levels.

In some preferred embodiments, this disclosure provides the following aspects:
1. A method of reducing body weight in a human being with fatty liver disease, the method comprising administering pemvidutide once weekly in an amount from at least about 1.2 to about 2.4 mg, optionally about 1.8 mg up to about 2.4 mg, or about 1.2 mg, about 1.8 mg, or about 2.4 mg, to the human being in need thereof for at least about 12 weeks and/or up to and including at least about 24 weeks; wherein the human being optionally has type 2 diabetes and/or optionally wherein the fatty liver disease is non-alcoholic fatty liver disease (NAFLD) or non-alcoholic steatohepatitis (NASH).
2. The method of aspect 1, wherein the body weight of the human being is reduced by at least 3% from baseline at week 12.
3. The method of aspect 1, wherein the body weight of the human being is reduced by at least 4% from baseline at week 12.
4. The method of aspect 1, wherein the pemvidutide is administered once weekly for 24 weeks in an amount of 1.2 mg.
5. The method of aspect 1, wherein the pemvidutide is administered once weekly in an amount of 1.8 mg.
6. The method of any preceding aspect, wherein the pemvidutide is administered once weekly in an amount of 2.4 mg.
7. The method of aspect 1, wherein a steady state dose is achieved after a dose escalating phase having a duration of about 2 weeks, about 3 weeks or about 4 weeks.
8. The method of aspect 1, wherein the human being suffers from type 2 diabetes.
9. The method of aspect 1, wherein the human being does not suffer from type 2 diabetes.
10. The method of aspect 1, wherein the human being has a body mass index (BMI kg/m²) of at least 28.
11. The method of aspect 1, wherein the human being has a body mass index (BMI kg/m²) of 30 or greater.
12. The method of aspect 1, wherein the human being has a level of liver fat as measured by MRI-PDFF of 10% or greater.
13. The method of aspect 1, wherein an absolute reduction in liver fat as measured by MRI-PDFF is a reduction of about 8% to about 15% after 12 weeks of the once weekly dosing.
14. The method of aspect 1, wherein the relative reduction in liver fat as measured by MRI-PDFF compared to baseline is about 40% to about 70% after 12 weeks of the once weekly dosing.
15. The method of aspect 1, wherein the relative reduction in liver fat as measured by MRI-PDFF compared to baseline is about 30% to about 50% after 24 weeks of the once weekly dosing, wherein the relative reduction is statistically significant defined as p<0.001 or p<0.0001.
16. The method of aspect 1, wherein the 1.8 mg and 2.4 mg once weekly doses induce at least 40% reduction in liver fat after 24 weeks wherein the reduction is significant defined as p<0.001 or p<0.01 as compared to placebo.
17. The method of aspect 1, wherein defatting of the liver as measured by MRI-PDFF compared to baseline is about 30% after 24 weeks of once weekly dosing of about 1.8 mg pemvidutide as compared to baseline in the human being.
18. The method of aspect 1, wherein liver volume as measured by MRI-PDFF compared to baseline is reduced after 24 weeks of once weekly dosing of about 1.2 mg, about 1.8, or about 2.4 mg pemvidutide as compared to placebo.
19. The method of aspect 1, wherein alanine aminotransferase (ALT) is reduced after 24 weeks of once weekly dosing of about 1.2 mg, about 1.8 mg, or about 2.4 mg as compared to placebo, optionally wherein the human being has baseline ALT of >30 IU/L before dosing.
20. The method of aspect 1, wherein the reduction in iron-corrected T1 is superior to 80 ms in about 80% of subjects, optionally wherein the reduction is significant defined as p<0.05 or p<0.005.
21. The method of aspect 1, wherein weekly dosing of about 1.2 mg, about 1.8 mg, or about 2.4 mg per week for 24 weeks results in weight loss in non-diabetic and/or diabetic patients as compared to placebo.
22. The method of aspect 1, wherein weekly dosing of about 1.2 mg, about 1.8 mg, or about 2.4 mg per week for 24 weeks results in in serum lipid levels as compared to placebo.
23. The method of aspect 1, wherein weekly dosing of about 1.2 mg, about 1.8 mg, or about 2.4 mg per week for 24 weeks reduced blood pressure without significantly increasing heart rate, optionally wherein systolic blood pressure is significantly reduced as defined as p<0.05 by the 2.4 mg weekly dose.
24. The method of aspect 1, wherein weekly dosing of about 1.2 mg, about 1.8 mg, or about 2.4 mg per week for 24 weeks improves glycemic parameters, optionally wherein the glycemic parameters are a reduction of fasting glucose and/or HbAlc levels.
25. A method of reducing liver fat content as determined by MRI-PDFF in a human being with fatty liver disease, the method comprising administering Pemvidutide once weekly for at least 12 continuous weeks in an amount from at least about 1.2 mg up to about 2.4 mg to the human being, wherein the human being:
   has been diagnosed with fatty liver disease that is non-alcoholic fatty liver disease (NAFLD) or non-alcoholic steatohepatitis (NASH);
   has a body mass index (BMI kg/m²) of greater than about 28; and,
   has a liver fat content of at least about 10% as measured by MRI-PDFF.
26. The method of any aspect herein, wherein a dose of about 1.2 mg per week induces in a population of human beings an absolute reduction in liver fat content of at least about 7%, optionally at least about 8%, that is significant (p<0.001) as compared to placebo at week 12.
27. The method of any aspect herein, wherein a dose of about 1.8 mg per week induces in a population of human beings an absolute reduction in liver fat content of at least about 12.5%, optionally at least about 14%, that is significant (p<0.001) as compared to placebo at week 12.
28. The method of any aspect herein, wherein a dose of about 2.4 mg per week induces in a population of human beings an absolute reduction in liver fat content of at least about 10%, optionally at least about 11%, that is significant (p<0.001) as compared to placebo at week 12.
29. The method of any aspect herein, wherein a dose of about 1.2 mg per week induces in a population of human beings a relative reduction in liver fat content of at least about 40%, optionally at least about 45%, that is significant (p<0.001) as compared to placebo at week 12.
30. The method of any aspect herein, wherein a dose of about 1.8 mg per week induces in a population of human beings a relative reduction in liver fat content of at least about 60%, optionally at least about 65%, that is significant (p<0.001) as compared to placebo at week 12.
31. The method of any aspect herein, wherein a dose of about 2.4 mg per week induces in a population of human beings a relative reduction in liver fat content of at least about 50%, optionally at least about 55%, that is significant (p<0.001) as compared to placebo at week 12.
32. The method of any aspect herein, wherein a dose of about 1.2 mg per week induces in at least about 55%, optionally at least about 60%, of a population of human beings at least about a 30% reduction in liver fat content that is significant (p<0.001) as compared to placebo at week 12.
33. The method of any aspect herein, wherein a dose of about 1.8 mg per week induces in at least about 80%, optionally at least about 90%, of a population of human beings at least about a 30% reduction in liver fat content that is significant (p<0.001) as compared to placebo at week 12.
34. The method of any aspect herein, wherein a dose of about 2.4 mg per week induces in at least about 75%, optionally at least about 85%, of a population of human beings at least about a 30% reduction in liver fat content that is significant (p<0.001) as compared to placebo at week 12.
35. The method of any aspect herein, wherein a dose of about 1.2 mg per week induces in at least about 35%, optionally at least about 40%, of a population of human beings at least about a 50% reduction in liver fat content that is significant (p<0.001) as compared to placebo at week 12.
36. The method of any aspect herein, wherein a dose of about 1.8 mg per week induces in at least about 60%, optionally at least about 70%, of a population of human beings about a 50% reduction in liver fat content that is significant (p<0.001) as compared to placebo at week 12.
37. The method of any aspect herein, wherein a dose of about 2.4 mg per week induces in at least about 60%, optionally at least about 70%, of a population of human beings at least about a 50% reduction in liver fat content that is significant (p<0.001) as compared to placebo at week 12.
38. The method of any aspect herein, wherein a dose of about 1.2 mg per week induces liver fat content normalization to less than or equal to about 5% liver fat content in at least about 20% of a population of human beings that is significant (p<0.05) as compared to placebo at week 12.
39. The method of any aspect herein, wherein a dose of about 1.8 mg per week induces liver fat content normalization to less than or equal to about 5% liver fat content in at least about 50%, optionally at least about 55%, of a population of human beings that is significant (p<0.0001) as compared to placebo at week 12.
40. The method of any aspect herein, wherein a dose of about 2.4 mg per week induces liver fat content normalization to less than or equal to about 5% liver fat content in at least about 50% of a population of human beings that is significant (p<0.001) as compared to placebo at week 12.
41. A method of inducing weight loss in a human being with fatty liver disease, the method comprising administering pemvidutide once weekly for at least 12 continuous weeks in an amount from at least about 1.2 mg up to about 2.4 mg to the human being, wherein the human being:
   has been diagnosed with fatty liver disease that is non-alcoholic fatty liver disease (NAFLD) or non-alcoholic steatohepatitis (NASH);
   has a body mass index (BMI kg/m²) of greater than about 28;
   optionally has been diagnosed with type 2 diabetes; and, has a liver fat content of at least about 10% as measured by MRI-PDFF.
42. The method of aspect 31, wherein the human being has not been diagnosed with type 2 diabetes and a dose of about 1.2 mg per week reduces the body weight of the human being by at least about 2.5%, optionally at least about 3%, that is significant (p<0.001) as compared to placebo at week 12.
43. The method of any aspect herein, wherein the human being has not been diagnosed with type 2 diabetes and a dose of about 1.8 mg per week reduces the body weight of the human being by at least about 4%, optionally about 5%, that is significant (p<0.001) as compared to placebo at week 12.
44. The method of any aspect herein, wherein the human being has not been diagnosed with type 2 diabetes a dose of about 2.4 mg per week reduces the body weight of the human being by at least about 2.5%, optionally about 3.5%, that is significant (p<0.001) as compared to placebo at week 12.
45. The method of any aspect herein, wherein the human being has been diagnosed with type 2 diabetes and a dose of about 1.2 mg per week reduces the body weight of the human being by at least about 2%, optionally at least about 3%, at week 12 that is significant (p<0.05) as compared to placebo at week 12.
46. The method of any aspect herein, wherein the human being has been diagnosed with type 2 diabetes and a dose of about 1.8 mg per week reduces the body weight of the human being by about 2.5%, optionally at least about 3.5%, that is significant (p<0.005) as compared to placebo at week 12.
47. The method of any aspect herein, wherein the human being has been diagnosed with type 2 diabetes and a dose of about 2.4 mg per week reduces the body weight of the human being by about 3%, optionally at least about 4%, that is significant (p<0.001) as compared to placebo at week 12.
48. The method of any aspect herein, wherein a dose of about 1.2 mg per week reduces the body weight of a population of human beings by at least about 3% that is significant (p<0.001) as compared to placebo at week 12.
49. The method of any aspect herein, wherein a dose of about 1.8 mg per week reduces the body weight of a population of human beings by at least about 4% that is significant (p<0.001) as compared to placebo at week 12.
50. The method of any aspect herein, wherein a dose of about 2.4 mg per week reduces the body weight of a population of human beings by at least about 3%, optionally at least about 3.5%, that is significant (p<0.001) as compared to placebo at week 12.
51. The method of any aspect herein wherein a dose of about 1.2 mg per week induces ALT reduction of at least about 11% in a population of human beings at week 12.
52. The method of any aspect herein wherein a dose of about 1.8 mg per week induces ALT reduction of at least about 13% in a population of human beings that is significant (p<0.05) as compared to placebo at week 12.
53. The method of any aspect herein wherein a dose of about 2.4 mg per week induces ALT reduction of at least about 13% in a population of human beings that is significant (p<0.05) as compared to placebo at week 12.
54. The method of any aspect herein wherein a dose of about 1.2 mg per week induces ALT reduction of at least about 18% at week 12 in a population of human beings with a baseline ALT of greater than or equal to about 30 IU/L.
55. The method of any aspect herein wherein a dose of about 1.8 mg per week induces ALT reduction of at least about 20% at week 12 in a population of human beings with a baseline ALT of greater than or equal to about 30 IU/L.
56. The method of any aspect herein wherein a dose of about 2.4 mg per week induces ALT reduction of at least about 20%, optionally at least about 25%, at week 12 that is significant (p<0.005) as compared to placebo in a population of human beings with a baseline ALT of greater than or equal to about 30 IU/L.
57. The method of any aspect herein, wherein the pemvidutide is administered by parenteral injection.
58. The method of any aspect herein, wherein the Pemvidutide is administered by subcutaneous injection.
59. The method of any aspect herein, wherein the human being has a body mass index (BMI kg/m²) of at least 28.
60. The method of any aspect herein, wherein the human being has a body mass index (BMI kg/m²) of 30 or greater.
61. The method of any preceding aspect, wherein the human being has level of liver fat as measured by MRI-PDFF of 10% or greater before the once weekly dosing.
62. The method of any aspect herein, wherein the absolute reduction in liver fat as determined using MRI-PDFF is from about 8% to about 15% after 12 weeks of the once weekly dosing.
63. The method of any aspect herein, wherein the liver fat content normalization to less than or equal to 5% is greater than from 20% to about 55% of the population after 12 weeks of once weekly dosing.
64. The method of any aspect herein, wherein the relative reduction in liver fat as determined using MRI-PDFF to baseline is greater than from about 40% to about 60% after 12 weeks of the once weekly dosing.
65. The method of any aspect herein, wherein the ALT reduction is greater than about 13% to about 25% of the population after 12 weeks of once weekly dosing.
66. The method of any aspect herein, wherein a steady state dose is achieved after a dose escalating phase having a duration of from two to four weeks.
67. The method of any aspect herein, wherein the pemvidutide is administered from a liquid formulation comprising at least about 2.5 mg/ml pemvidutide.
68. The method of any aspect herein, wherein the administration of pemvidutide induces a significant reduction in serum lipids and/or atherogenic small- to medium-sized LDL particle concentration.
69. The method of any aspect herein wherein the atherogenic small- to medium-sized LDL particle concentrations are reduced by at least −0.2 log₂ fold change relative to placebo following 43 and/or 84 days of administration of pemvidutide.
70. The method of any aspect herein wherein the total serum triglyceride concentration of the subject is reduced by at least −0.2 log₂ fold change relative to placebo following 43 and/or 84 days of administration of pemvidutide when measured by 2D-NMR.
71. The method of any aspect herein wherein the serum lipids are selected from glycerolipids, sterols, glycerophospholipids, and sphingolipids, optionally wherein the reductions are at least −0.2 log 2 fold change.
72. The method of any aspect herein wherein a reduction in serum phosphatidylethanolamine, phosphatidylcholine, lysophosphatidylethanolamine, and/or lysophosphatidylcholine follows dosing with pemvidutide.
73. A liquid pharmaceutical formulation comprising: SEQ ID NO: 1 and about 0.020% (w/w) polysorbate 20, about 0.348% (w/w) arginine, and about 4.260% (w/w) mannitol in deionized water (pH 7.7±0.1).
74. The formulation of aspect 73, comprising 1.8 mg of SEQ ID NO: 1 as a therapeutic dose.
75. The formulation of aspect 74, wherein the therapeutic dose induces weight loss in a subject in need thereof.

Other aspects of this disclosure are also contemplated as will be understood by those of ordinary skill in the art.

Unless defined otherwise or clearly indicated otherwise by their use herein, all technical and scientific terms used herein have the same meaning as commonly understood by those of ordinary skill in the art to which this application belongs. As used in the specification and the appended claims, the word “a” or “an” means one or more. As used herein, the word “another” means a second or more. The acronym “aka” means also known as. The term “exemplary” as used herein means “serving as an example, instance or illustration”. Any embodiment or feature characterized herein as “exemplary” is not necessarily to be construed as preferred or advantageous over other embodiments or features. In some embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Optional or optionally means that the subsequently described event or circumstance can or cannot occur, and that the description includes instances where the event or circumstance occurs and instances where it does not. All publications, patents, and patent applications mentioned in this specification are herein incorporated by reference in their entireties to the same extent as if each individual publication, patent, or patent application was specifically and individually indicated to be incorporated by reference.

Certain embodiments are further described in the following examples. These embodiments are provided as examples only and are not intended to limit the scope of the claims in any way.

### EXAMPLES

### Example 1. A Phase 1, 12-Week, Randomized, Double-Blind, Placebo-Controlled Study of ALT-801 (Pemvidutide) in Diabetic and Non-Diabetic Overweight and Obese Subjects with Non-Alcoholic Fatty Liver Disease (NAFLD)

Provided herein is disclosure of a Phase 1, multicenter, randomized, double-blind, placebo-controlled study to assess the effects of Pemvidutide (also termed herein as ALT-801), the safety of Pemvidutide and effects on hepatic fat fraction, anthropometric parameters, lipid metabolism, inflammatory markers and fibrosis markers in diabetic and non-diabetic (type 2 diabetes) overweight and obese (BMI 28.0 kg/m²) subjects with NAFLD. This study was designed to assess changes in hepatic fat fraction by MRI-PDFF (magnetic resonance imaging-proton density fat fraction) in diabetic and non-diabetic overweight and obese subjects with NAFLD after 12 weeks of ALT-801 treatment. The trial was conducted without adjunctive diet and exercise interventions. The study assessed changes in body weight, lipid metabolism, metabolic markers, and inflammatory markers and safety and tolerability of ALT-801 after 12 weeks of treatment. Subjects were stratified for the presence or absence of diabetes at baseline. Fibroscan and cT1 assessments evaluated changes in liver inflammatory and fibrotic activity at the end of 12 weeks of treatment.

Overweight and obese volunteers with BMI ≥28 kg/m² were studied because this is the typical population of individuals with NAFLD, and these subjects are able to better tolerate the predicted Pharmacodynamics (PD) effect of weight loss and could even benefit from treatment. A hepatic fat fraction of 10% has been typical for the evaluation of hepatic fat fraction in subjects with NAFLD. In other words, individuals with a liver fat fraction of 10% or greater are typically diagnosed with NAFLD. Diabetic subjects that require insulin, sulfonylureas or DDP-4 inhibitors for control of diabetes were excluded in this study. Exclusions were instituted that might otherwise affect an accurate assessment of the effects of ALT-801 on safety or PD. The BMI upper limit is set to 45 kg/m² as subjects over this BMI are no likely to fit into the MRI scanner.

A Fibroscan value of <10 kPa adequately excludes subjects with advanced fibrosis, who are not considered to be appropriate candidates for this clinical trial.

The study, described in this example, was carried out in 94 subjects overweight and obese (body mass index [BMI] ≥28.0 kg/m²) Type 2 diabetic and non-diabetic subjects with nonalcoholic fatty liver disease (NAFLD) over about 4.5 months, including up to a 35-day screening period, and 85-day treatment period and a 25-day follow-up period, with treatment with ALT-801 (a composition comprising SEQ ID NO.: 1, “study medication”) or placebo administered by subcutaneous (SC) injection once weekly for up to twelve (12) doses.

The baseline characteristics of the subjects included in this study are shown in Table 5 below:

TABLE 5

| | | Treatment | | | | | | | |
|---|---|---|---|---|---|---|---|---|---|
| | | Placebo | | 1.2 mg | | 1.8 mg | | 2.4 mg | |
| Characteristic | | (n = 24) | | (n = 23) | | (n = 23) | | (n = 24) | |
| Age, years | Mean (SD) | 47.89 | (14) | 48.6 | (11) | 50.3 | (9) | 48.8 | (8) |
| Sex | Female, n (%) | 14 | (58.3%) | 9 | (39.1%) | 12 | (52.2%) | 15 | (62.5%) |
| Race | White, n (%) | 21 | (87.5%) | 21 | (91.3%) | 20 | (87.0%) | 24 | (100%) |
| | Other, n (%) | 3 | (12.5%) | 2 | (8.7%) | 3 | (13.0%) | 0 | (0.0%) |
| Ethnicity | Hispanic, n (%) | 14 | (58.3%) | 20 | (87.0%) | 19 | (82.6%) | 18 | (75.0%) |
| | Not Hispanic, | 10 | (41.7%) | 3 | (13.0%) | 4 | (17.4%) | 6 | (25.0%) |
| | n (%) | | | | | | | | |
| BMI, kg/m² | Mean (SD) | 36.9 | (4.7) | 36.3 | (5.6) | 35.4 | (3.9) | 35.3 | (5.0) |
| Body weight, kg | Mean (SD) | 105.1 | (20.8) | 102.4 | (14.6) | 98.9 | (19.7) | 98.2 | (18.9) |
| Diabetes status | T2D, n (%) | 6 | (25.0%) | 7 | (30.4%) | 7 | (30.4%) | 7 | (33.3%) |
| Liver fat content | Mean (SD) | 23.8 | (9.2) | 21.57 | (7.3) | 21.8 | (8.0) | 20.2 | (7.0) |
| (LFC), % | | | | | | | | | |
| ALT, IU/L | Mean (SD) | 39.5 | (21.4) | 32.4 | (13.8) | 36.4 | (15.6) | 37.8 | (24.4) |
| Blood pressure, | Systolic, | 122.8 | (11.4) | 129.0 | (14.1) | 123.2 | (15.9) | 125.9 | (12.3) |
| mm Hg | mean (SD) | | | | | | | | |
| | Diastolic, | 79.6 | (6.0) | 79.3 | (9.1) | 77.8 | (9.7) | 80.1 | (8.6) |
| | mean (SD) | | | | | | | | |
| Total cholesterol, | Mean (SD) | 181.4 | (39.0) | 186.9 | (44.8) | 200.0 | (35.2) | 182.2 | (39.7) |
| mg/dL | | | | | | | | | |
| LDL cholesterol, | Mean (SD) | 100.0 | (38.2) | 100.2 | (34.3) | 116.6 | (33.6) | 101.3 | (33.0) |
| mg/dL | | | | | | | | | |
| Triglycerides, | Mean (SD) | 169.3 | (90.1) | 224.9 | (119.1) | 192.2 | (114.9) | 220.0 | (169.3) |
| mg/dL | | | | | | | | | |
| HDL cholesterol, | Mean (SD) | 47.5 | (6.8) | 42.6 | (9.1) | 47.0 | (9.9) | 45.3 | (7.3) |
| mg/dL | | | | | | | | | |

The safety objective of this study was to assess the safety and tolerability of ALT-801 in 18-65 years old subjects with NAFLD (NAFLD without significant fibrosis, defined as FibroScan® controlled attenuation parameter (CAP) ≥280 dB/m and liver stiffness measurement (LSM)<10 kPa (i.e., indicating the absence of significant fibrosis), magnetic resonance imaging derived proton density fat fraction (MRI-PDFF) ≥10%), hemoglobin Alc (HbAlc) of less than 9.5%, and alanine aminotransferase (ALT) or aspartate aminotransferase (AST) values of ≤75 IU/ml. The pharmacodynamic (PD) objectives of this study were to evaluate the effects of ALT-801 on hepatic fat fraction, anthropometric parameters, lipid metabolism, metabolic markers, inflammatory markers, fibrosis markers, and lipotoxicity markers. The pharmacokinetic (PK) objectives of this study were to evaluate the effects of ALT-801 and metformin exposure (drug interactions) on the subjects (e.g., ALT-801 and metformin concentrations in blood over time).

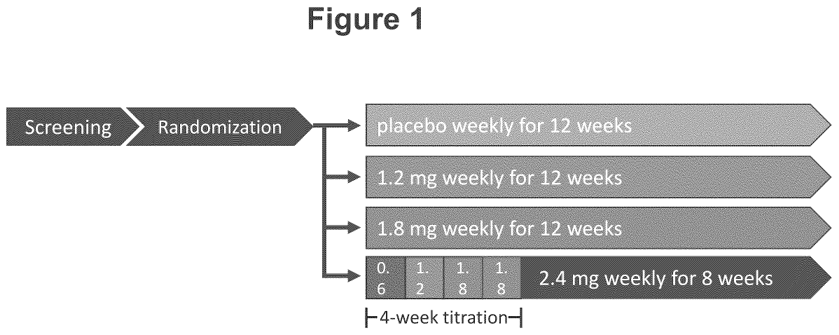

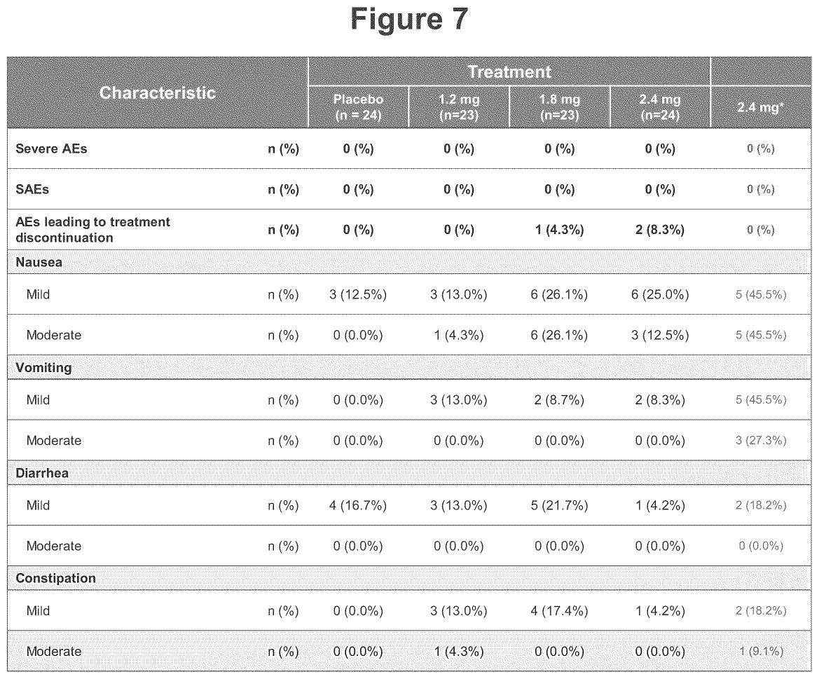

After providing informed consent, subjects underwent a screening period of up to 35 days. Subjects were instructed on how to maintain their normal diets, alcohol consumption and physical activities and not to start any new diets, supplements, or exercise programs at any time while participating in the study. Counseling was provided on diet and exercise on the Day 1 visit and reinforced at subsequent visits. Study subjects were randomized 1:1:1:1 to one of the following treatment arms: 1) ALT-8011.2 mg SC once weekly for 12 weeks; 2) ALT-8011.8 mg SC once weekly for 12 weeks; 3) ALT-801 0.6 mg SC at week 1, 1.2 mg SC at week 2, 1.8 mg SC once weekly for 2 weeks (weeks 3 and 4), and 2.4 mg SC once weekly for weeks 5 through 12; or, 4) placebo SC once weekly for 12 weeks. See FIG. 1. Subjects were stratified by presence or absence of diabetes at baseline. Subjects in the treatment arm with a final therapeutic dose of 2.4 mg underwent a rapid 4-week dose escalation to improve tolerability and reduce the possibility of adverse events. A previous study conducted with pemvidutide, first in man study, showed that the 2.4 mg dose had a relatively high occurrence of certain adverse events. See FIG. 7, second 2.4 mg column as compared to the first 2.4 mg column (this study). See WO 2022/125598. Accordingly provided herein is a rapid dose escalation regimen of pemvidutide with an initial one week dose of 25% of the total therapeutic dose; a second week dose of 50% of the total therapeutic dose; and a third and fourth week dose each of 75% of the total therapeutic dose. The fifth and subsequence weeks the subjects are administered 100% of the total therapeutic dose. In certain embodiments the dose escalation regimen is 0.6 mg, 1.2 mg, and 1.8 mg for a final therapeutic dose of 2.4 mg.

Subjects received the first dose of study medication on Day 1 (“baseline”). Subsequent visits were conducted weekly, at the clinic, home or work, through Day 85 or early termination. Subjects returned for a safety follow-up visit on Day 110. Investigators followed the decision criteria for the timing and method of intervention in subjects who developed worsening abnormal liver function tests during the 12-week treatment period. Fasting glucose levels were measured by a glucometer and documented by study staff at baseline and prior to each dose. On non-visit days, subjects were monitored to record fasting glucose each morning and contacted the study site for a reading >240 mg/dL or <70 mg/dL. Subjects were also educated on symptoms and treatment of hypoglycemia and to obtain a glucometer reading if they experience glucose <70 mg/mL or symptoms suggestive of hypoglycemia. Subjects recorded any symptoms of hypoglycemia experienced at home in a log, which was be reviewed by the Investigator at each visit commencing with Day 8. Investigators counseled subjects on how to keep their fasting glucose within the limits, including repeated diet counseling, and followed trial decision criteria for the timing and method of intervention in subjects with persistent hyperglycemia during the 12-week treatment period. Certain subjects who exhibited a significant decrease of fasting glucose was repeatedly observed (<50 mg/dL). Sparse blood samples were collected for ALT-801 PK to be combined with data from other studies in population PK and PK-PD modeling and for metformin PK to assess the change in metformin concentrations over time in the presence of ALT-801. Blood samples were also collected to assess immunogenicity.

Power and Sample Size Assumptions used in this study were that the sample size was considered adequate to meet the safety assessments of a Phase 1 study. Based on treatment effects observed in prior studies of NAFLD, the study also had adequate power to detect meaningful differences in the change in hepatic fat fraction by MRI-PDFF compared to baseline in the subjects with ALT-801 compared to subjects who received placebo SC injection at a 0.05 level of significance (2-sided).

For the statistical analysis, all randomized subjects who receive at least one dose of study medication (Safety Population) were included in the safety analyses. The assessments of the secondary and PD endpoints were conducted in the PD population, which consisted of all randomized subjects who receive at least one dose of study medication and who had results from baseline and at least one post-baseline PD assessment.

Two interim analyses were performed: 1) when all subjects complete the Day 43 visit; and 2) when all subjects complete the Day 85 visit. For these analyses, unblinding was restricted to the treatment group level, and the study team remain blinded to individual treatment assignments. Analyses included safety, weight loss and MRI-PDFF since the start of treatment and available PK data. Summary data by study part, dose level, treatment group (active or placebo), and day where applicable were reported. Continuous safety data was summarized with descriptive statistics (arithmetic mean, standard deviation [SD], median, minimum, and maximum) by dose level and treatment (active or placebo). Categorical safety data was summarized with frequency counts and percentages by dose level, treatment group, and day where applicable. AEs were coded using the most current Medical Dictionary for Regulatory Activities (MedDRA) version. A by-subject AE data listing, including verbatim term, preferred term, system organ class (SOC), treatment, severity, and relationship to study medication, was provided. The number of subjects experiencing treatment-emergent AEs (TEAEs) and number of individual TEAEs, and injection site reactions were summarized by treatment group, SOC and preferred term. TEAEs were also be summarized by severity and by relationship to study medication. Laboratory evaluations, including liver function tests and fasting glucose, vital signs (including calculation of RPP), and ECG assessments were summarized by treatment group, dose levels, and protocol specified collection time point. A summary of change from baseline at each protocol specified time point by treatment group was also determined. Changes in physical examinations will be listed for each subject. Concomitant medications were listed by subject and coded using the most current version of the World Health Organization (WHO) Drug Dictionary. Medical histories were coded using the most current MedDRA version and will be listed by subject.

For pharmacodynamics determinations, descriptive statistics, including the numbers and percentages for categorical variables and the numbers, means, SDs, medians, minimums, and maximums for continuous variables were provided by dose level and treatment (ALT-801 or placebo), and by day when applicable. Changes from baselines in hepatic fat fraction, anthropometric parameters, lipid metabolism, metabolic markers, lipotoxicity markers, and inflammation markers were summarized by treatment group and strata with descriptive statistics (sample size [N], arithmetic mean, SD, median, minimum, maximum, geometric mean, and geometric coefficient of variation [CV %]). The effects of baseline BMI on PD parameters were evaluated by covariate analyses. Inferential statistics were performed, as applicable. All analyses were described in a statistical analysis plan (SAP). The changes in hepatic fat fraction by MRI-PDFF, body composition and liver inflammatory and fibrotic markers by MRI and Fibroscan, and other continuous variables were compared between ALT-801 and placebo groups using the analysis of covariance (ANCOVA) test, where treatment arm as a factor and the stratification of presence or absence of diabetes or the corresponding baseline demographic characteristics (gender, race, BMI) as covariates. The Cochrane Mantel Haenszel test will be applied for secondary endpoints that are categorical in nature, while considering the stratification of presence or absence of diabetes, at a one-sided significance level of 0.025.

Quality of Life was also measured as changes from baseline in the two summary scores for physical health and mental health, and 8 domain scores for SF-36 and the composite score for the IWQoL-Lite for CT will be listed and summarized by treatment group with descriptive statistics (N, arithmetic mean, SD, median, minimum, maximum, geometric mean, and geometric CV %). Inferential statistics applicable to continuous endpoints were applied, as described above.

For pharmacokinetics (PK) determinations, individual ALT-801 and metformin concentration data were listed and summarized by treatment group and timepoint with descriptive statistics (N, arithmetic mean, SD, CV %, median, minimum, and maximum). Individual and mean±SD ALT-801 concentration-time profiles for each cohort were also be presented graphically. Change from baseline metformin concentrations were also listed and summarized by treatment group and timepoint with descriptive statistics (N, arithmetic mean, SD, CV %, median, minimum, and maximum). A population PK model was developed to enable the prediction of individual subject ALT-801 plasma concentration time curves and related PK parameters. Covariates, including sex, age, body weight, BMI, and concomitant medications were explored wherever possible and exposure response relationships will be explored for efficacy and safety endpoints where possible. This analysis combined data from multiple studies and will be done as a separate study and report.

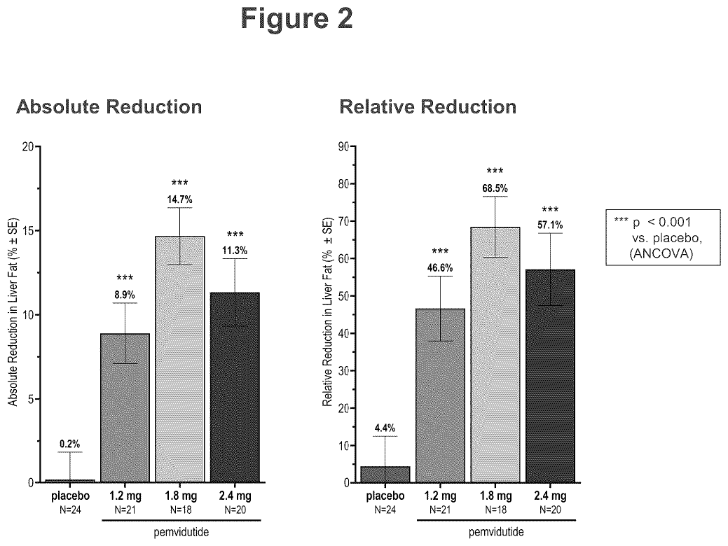

As shown in FIG. 2, ALT-801 induced a significant absolute reduction in liver fat across all non-placebo doses of about 10% or more (placebo: 0.2%; 1.2 mg: 8.9%; 1.8 mg dose: 14.7%; and 2.4 mg dose: 11.3% (all doses, p<0.001 vs. placebo)), and a significant relative reduction in liver fat across all non-placebo doses of at least about 40% or more (placebo: 4.4%; 1.2 mg: 46.6%; 1.8 mg dose: 68.5%; and 2.4 mg dose: 57.1% (p<0.001 vs. placebo)) at week 12.

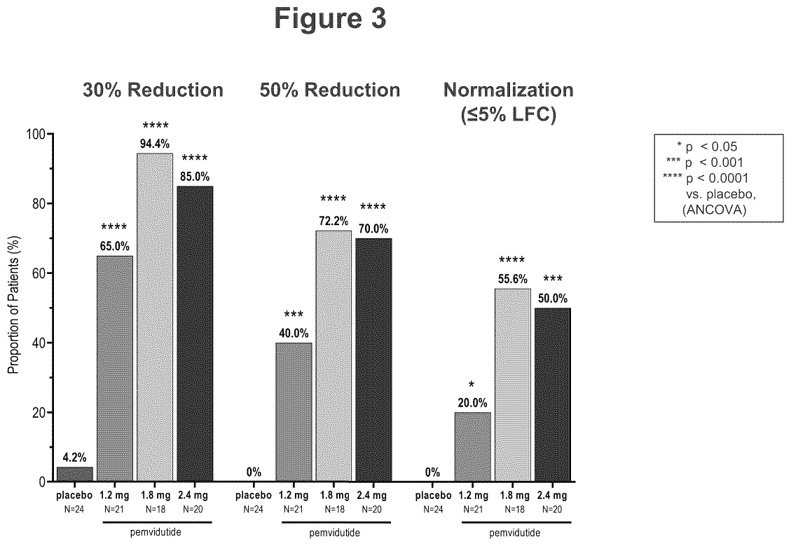

As shown in FIG. 3, ALT-801 achieved a at least 30% reduction in liver fat content as measured by MRI-PDFF at week 12 in a significant proportion of patients of subjects completing 12 weeks treatment (p<0.0001 vs. placebo) at all doses 1.2 mg dose: 65% of patients; 1.8 mg: 94.4% of patients; and 2.4 mg doses: 85% of patients) as compared to placebo (4.2% of patients) at week 12.

FIG. 3 also shows ALT-801 achieved at least a 50% reduction in liver fat content as measured by MRI-PDFF at week 12 in a significant proportion of patients of subjects completing 12 weeks treatment at all doses (1.2 mg dose: in about 40% of patients (p<0.001); 1.8 mg dose: 72.2% of patients at the 1.8 mg (p<0.0001); and 2.4 mg doses; 70% (p<0.0001)) as compared to placebo (0% of patients) at week 12.

FIG. 3 also shows ALT-801 achieved normalization in liver fat content (equal or below 5%) as measured by MRI-PDFF in a significant proportion of patients completing 12 weeks treatment at all doses 1.2 mg dose: 20% of patients (p<0.05); 1.8 mg dose: 55.6% patients (p<0.0001); 2.4 mg dose: 50.0% (p<0.001) as compared to placebo (0%) at week 12.

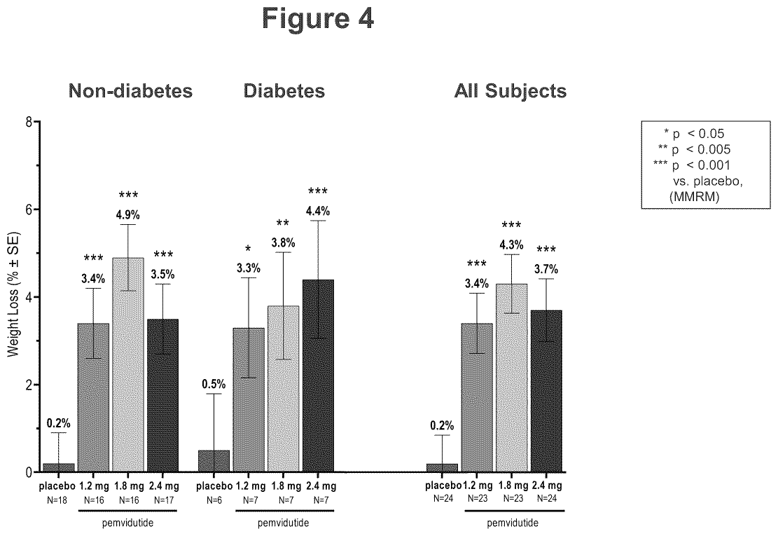

FIG. 4 shows ALT-801 induced significant weight loss at all doses in the non-diabetic subjects at the 1.2 mg dose of 3.4% (p<0.001); at the 1.8 mg dose of 4.9% (p<0.001); and at the 2.4 mg dose of 3.5% (p<0.001) as compared to placebo (0.2%) at week 12.

FIG. 4 also shows ALT-801 induced weight loss at all doses in the diabetic subjects at the 1.2 mg dose of 3.3% (p<0.005); at the 1.8 mg dose of 3.8% (p<0.05); and at the 2.4 mg dose of 4.4% (p<0.001) as compared to placebo (0.5%) at week 12.

FIG. 4 also shows ALT-801 induced significant weight loss at all doses in all subjects at the 1.2 mg dose of 3.4% (p<0.001); at the 1.8 mg dose of 4.3% (p<0.001); and at the 2.4 mg dose of 3.7% (p<0.001) as compared to placebo (0.2%) at week 12.

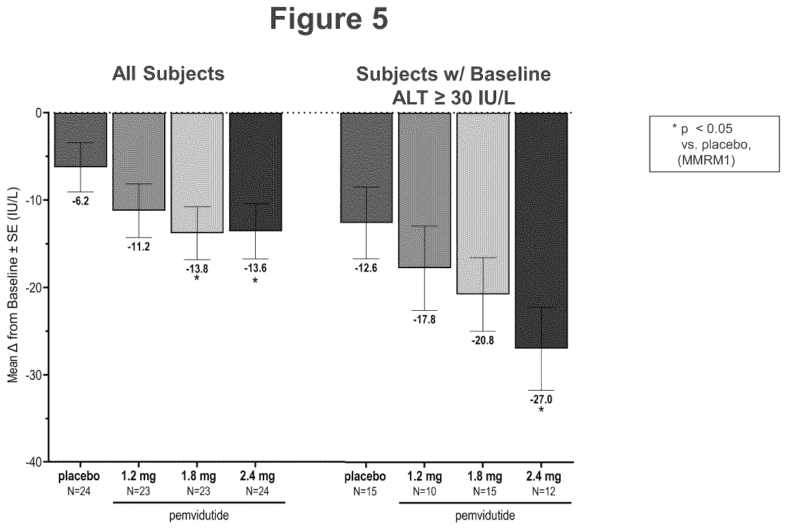

FIG. 5 shows ALT-801 induced ALT reduction of at least 10% in all subjects at the 1.2 mg dose (−11.2%); the 1.8 mg dose (−13.8%, p<0.05); and the 1.8 mg dose (−13.6%, p<0.05) in all subjects as compared to placebo (−6.2.%) at week 12.

FIG. 5 also shows ALT-801 induced significant ALT reduction in subjects with baseline ALT of greater than or equal to 30 IU/L at the 1.2 mg dose (−17.8%); the 1.8 mg dose (−20.8%); and the 2.4 mg dose (−27%, p<0.005) in all subjects (n=83) as compared to placebo (−12.6%) after week 12.

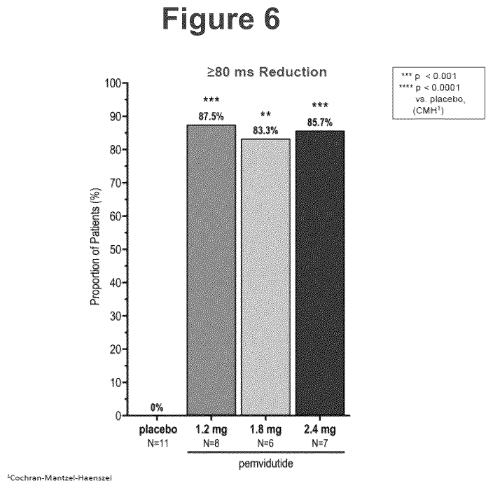

FIG. 6 shows ALT-801 induces the proportion of subjects showing ≥80 ms for Iron-corrected T1 (cT1) at the 1.2 mg dose (87.5%); the 1.8 mg dose (83.3%) and the 2.4 mg dose (85.7%) as compared to placebo (0%) after week 12.

Accordingly, provided herein is a method of using pemvidutide for reducing body weight in a human being with fatty liver disease, wherein the method comprises administering pemvidutide once weekly in an amount from at least 1.8 mg up to 2.4 mg to the human being in need thereof; and, wherein the human being may (or may not) suffer from type 2 diabetes and wherein the fatty liver disease is non-alcoholic fatty liver disease (NAFLD) or non-alcoholic steatohepatitis (NASH). In certain embodiments, provided herein is a method of using pemvidutide for reducing body weight (e.g., subject that is overweight or obese) in a human being with NAFLD or NASH, wherein the method comprises administering pemvidutide once weekly in an amount from at least 1.8 mg up to 2.4 mg to the human being in need thereof; and, wherein the human being does not have type 2 diabetes (e.g., non-diabetic).

Serum lipids were not significantly altered in the subjects following administration of ALT-801 after 12 weeks. Even without dose titration, the symptoms experienced by subjects after 12 weeks treatment with ALT-801 were predominantly mild and transient in nature, consistent with known GLP-1 class effects. No serious adverse events (AEs), severe AEs were observed after 12 weeks treatment with ALT-801. Also, AEs leading to treatment discontinuation were observed a very low rates following administration of ALT-801 for 12 weeks. In addition, mean serum alanine aminotransferase (ALT) levels declined in all subjects, and in subjects with baseline serum ALT above 30 IU/L, levels declined more than 17 IU/L at all dose levels and 27.0 IU/L in the 2.4 mg dose cohort. No clinically significant ALT elevations (defined as an increase to 3-fold or greater the upper limit of normal) were observed for 12 weeks. Glycemic control was unaffected, with no clinically meaningful changes in HbA1c or fasting glucose for 12 weeks.

In conclusion of this 12 weeks clinical study, this example shows that ALT-801 administration leads to robust (>60%) relative reductions in liver fat, superior to the effects of other GLP-1 agonists and leading NASH candidates. Regarding weight loss, this example shows placebo-adjusted weight loss (4.7%) superior to semaglutide (Wegovy; GLP-1 agonist) at 12 weeks in non-diabetic subjects, and placebo-adjusted weight loss (4.5%) superior to tirzepatide (GIP/GLP-1 agonist) at 12 weeks in diabetic subjects. The trial was conducted without adjunctive diet and exercise interventions that are the standard for obesity trials. In addition, no severe or serious AEs were observed (FIG. 7); no three-fold or greater ALT elevations were observed; very low rates of AE leading to treatment discontinuation including 1 (4.3%) in the 1.8 mg ALT-801 arm and 1 (4.2%) in the 2.4 mg ALT-801 arm, both secondary to gastrointestinal intolerance; and, only 1-3 beat-per-minute (bpm) increases in heart rate (HR) were observed, which is within the range of other GLP-1 based agents that have been used in human beings.

### Example 2. Effect of Pemvidutide (ALT-801), a GLP-1/Glucagon Dual Receptor Agonist, on Pathogenic Lipid Mediators

The potential for cardiovascular (CV) risk reduction through incretin-based therapy is receiving increased attention. pemvidutide is a long-acting GLP-1/glucagon (1:1) dual receptor agonist under development for treatment of NASH and obesity. pemvidutide combines the anorectic effects of GLP-1 receptor agonism (RA) with the increased energy expenditure and lipid-lowering effects of glucagon RA. Plasma lipids serve multiple functions in biological systems such as energy storage, metabolic regulation, signaling, proliferation, and apoptosis. The plasma lipidome can be analyzed using nuclear magnetic resonance (NMR) and ultra-high performance liquid chromatography mass spectrometry (UHPLC-MS).

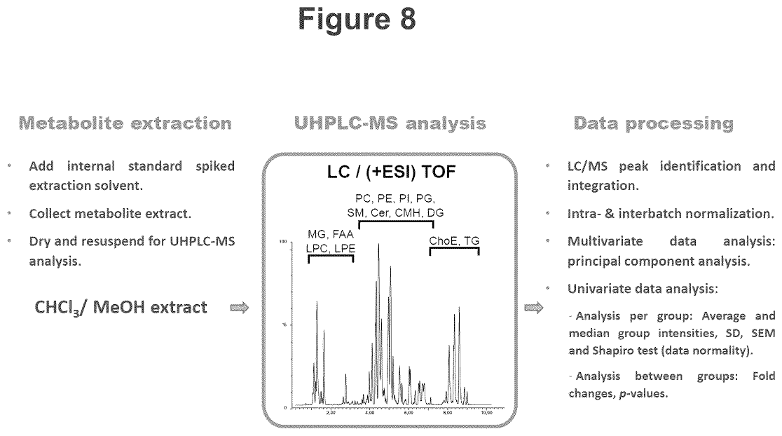

To test the lipid-lowering effects of pemvidutide, an analysis of Phase I clinical trial (NCT0456124) data was carried out. The subjects with overweight/obesity (BMI 25-40 kg/m²) were randomized at 1 site in Australia (NCT0456124). Subjects were randomized 4:1 pemvidutide:placebo, with placebos pooled. The pemvidutide doses were 1.2 mg, 1.8 mg, and 2.4 mg, administered weekly for twelve (12) weeks, with no dose titration or adjunctive lifestyle intervention (no diet or exercise interventions). pemvidutide was well-tolerated at all dose levels, without use of dose titration. All AEs in these groups were of mild or moderate severity, so no grade 3 (severe) AEs were seen here and no SAEs or AEs leading to treatment discontinuation were reported. Lipoprotein and glycoprotein profiling covering 33 lipoprotein related parameters was performed by ¹H-NMR on fasting plasma samples obtained at Day −1 (baseline), Day 43, and Day 84 from the 34 subjects who completed NCT0456124. Lipidomic profiling covering 600 lipid species was performed by ultra-high performance liquid chromatography-mass spectrometry on fasting plasma samples obtained at Day −1 (baseline), Day 43, and Day 84 from the 34 subjects who completed NCT0456124. For the lipid profiling, plasma fractionation was performed either using methanol to extract fatty acyls, bile acids, steroids, and lyso-glycerophospholipids, or using a chloroform/methanol mix to extract glycerolipids, cholesteryl esters, sphingolipids, and glycerophospholipids. Lipid classification followed the classification system proposed by Fahy et al. (J. Lipid Res. 2005; 46:839-861) and the LIPID MAPS initiative (http://www.lipidmaps.org) (see FIG. 8).

Consistent with the data presented in Example 1, the data presented here shows that Pemvidutide had favorable effects on weight loss, body mas index (BMI), blood pressure, total cholesterol, LDL cholesterol, triglycerides, and apoprotein B, as summarized in Table 6:

TABLE 6
Key pharmacodynamic endpoints (change from baseline)

| | | Treatment | | | | | | | |
|---|---|---|---|---|---|---|---|---|---|
| | | | | | | | | Pooled | |
| | | 1.2 mg | | 1.8 mg | | 2.4 mg | | placebo | |
| Endpoint | | (n = 7) | | (n = 9) | | (n = 11) | | (n = 7) | |
| Weight loss, % | mean (SEM) | −4.9 | (1.4) | −10.3 | (1.1)*** | −9.0 | (1.1)** | −1.6 | (1.4) |
| BMI, % | mean (SEM) | −4.8 | (1.22) | −10.4 | (1.14) | −7.0 | (1.12) | −0.8 | (1.37) |
| Systolic BP, % | mean (SEM) | −10.2 | (9.23) | −12.5 | (8.04) | −17.4 | (7.32) | −10.5 | (12.45) |
| Diastolic BP, % | mean (SEM) | −4.7 | (9.73) | −7.1 | (3.02) | −6.0 | (11.47) | −2.7 | (13.54) |
| Total | mean (SEM) | −20.0 | (2.8) | −28.0 | (3.4)* | −27.7 | (4.6) | −9.1 | (3.7) |
| cholesterol, % | | | | | | | | | |
| LDL cholesterol, % | mean (SEM) | −18.4 | (3.6) | −25.6 | (3.7)* | −23.9 | (6.4)* | −4.3 | (6.1) |
| HDL cholesterol, % | mean (SEM) | −16.7 | (1.9) | −30.3 | (3.2) | −36.0 | (3.8) | −19.2 | (5.7) |
| Triglycerides, % | mean (SEM) | −37.0 | (1.7)** | −37.9 | (7.7)*** | −29.2 | (7.7)* | −8.2 | (7.0) |
| Apoprotein B, % | mean (SEM) | −45.4 | (4.8) | −23.4 | (3.3) | −16.8 | (10.9) | −9.9 | (8.0) |

*p < 0.05,
**p < 0.01,
***p < 0.001, vs. placebo

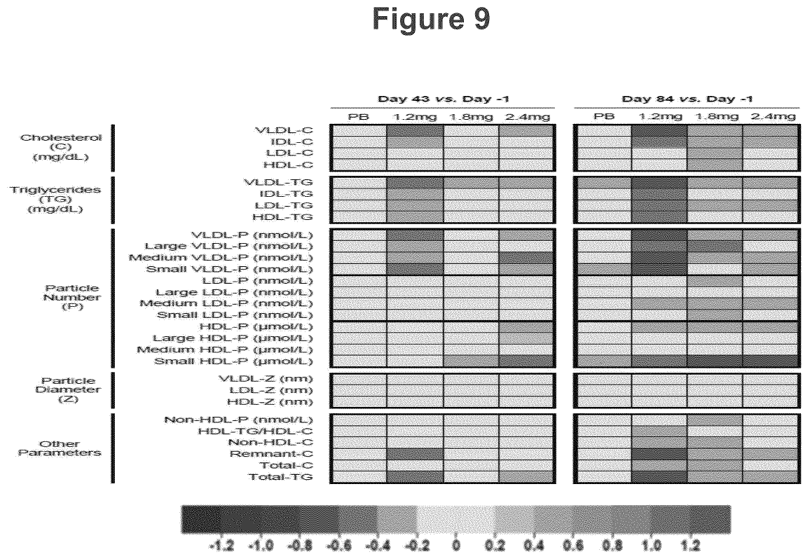

Pemvidutide also caused generally consistent and favorable changes in lipoprotein particle subspecies as determined by 2D-NMR analysis (see FIG. 9). The color code represents the log 2 (robust fold-change) with blue colors denoting reduced lipoproteins (negative fold-changes) and red colors denoting increased lipoproteins (positive fold-changes). FIG. 9 shows that the number of VLDL and LDL particles and the number of smaller HDL particles tend to be reduced following treatment with pemvidutide.

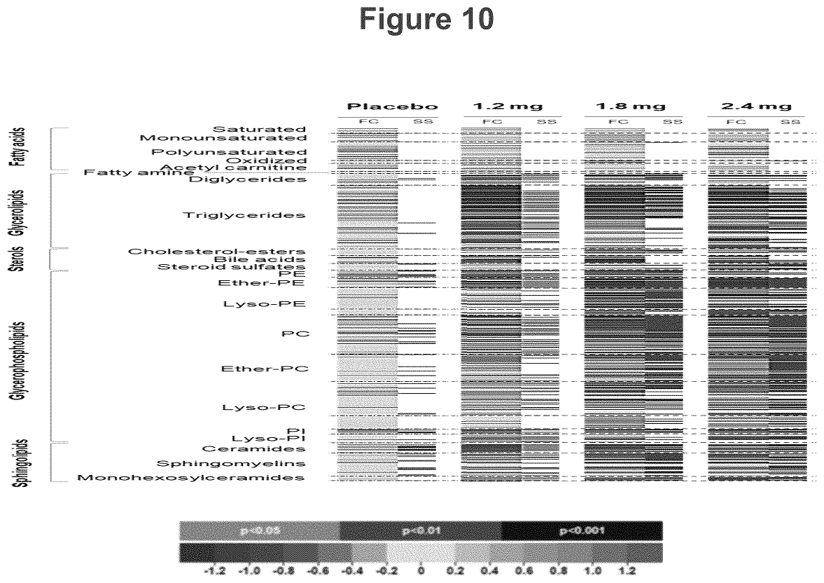

Given the observed changes in the total cholesterol, triglycerides and lipoproteins, an evaluation of the serum lipid composition covering 600 lipid species at Day −1 (baseline), Day 43, and Day 84 from the 34 subjects who completed NCT0456124 was performed. Results are presented in FIG. 10 as log 2 pairwise fold change with blue colors denoting a reduction in the lipid relative to baseline, and red an increase. The grey/black bars indicate significant p-values of Wilcoxon test. Within 12 weeks of treatment, pemvidutide significantly reduced serum lipid levels, especially glycerolipids (diglycerides and triglycerides), glycerophospholipids (phosphatidylethanolamines, phosphatidylcholines, lysophosphatidylethanolamines, and lysophosphatidylcholines) and sphingolipids (ceramides and sphingomyelins) associated with reduction in cardiovascular (CV) risk as well as reduction in insulin resistance (see FIG. 10). Thus, at Day 85, substantial and highly statistically significant reductions across multiple bioactive lipid classes were observed compared to placebo.

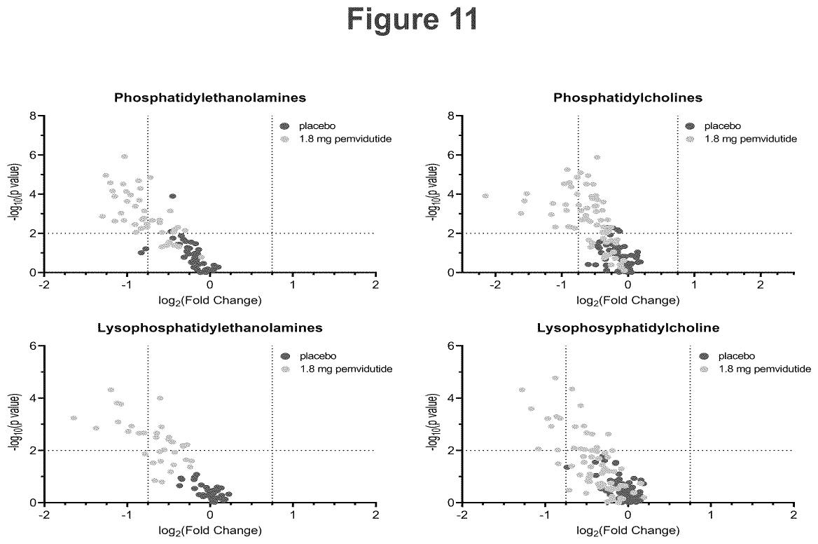

Based on this lipodomics data, volcano plots were generated as shown in FIG. 11, focusing on changes in atherogenic lipids following treatment with 1.8 mg pemvidutide. The horizontal dashed line represents the lower limit of significance, while the vertical lines show a fold-change from baseline of ±0.75 on the log 2 scale. Responses in the upper left quadrant indicate particularly meaningful changes in the atherogenic lipids. pemvidutide treatment significantly reduced pro-atherogenic lyso-PC levels (see FIG. 11), suggesting a potential for decreased oxidized LDL (Law et al. Int. J. Mol. Sci. 2019; 20(5):1149). pemvidutide treatment also significantly reduced atherosclerotic plaque forming phosphatidyl-ethanolamine (PE) glycerophospholipids level. Based on these findings, pemvidutide demonstrates promise as an agent for reducing cardiovascular (CV) risk.

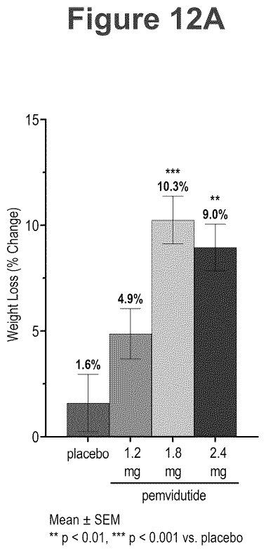

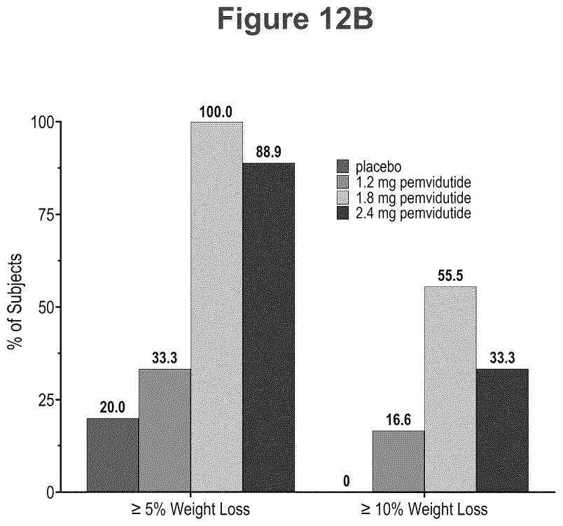

In this trial, pemvidutide induced substantial weight loss at week 12 as illustrated in FIGS. 12A and 12B. Body weight loses of up to a mean of 10.3% (8.7% placebo adjusted) at the 1.8 mg dose level were observed, with a high degree of statistical significance after just 12 weeks of weekly treatment. Additionally, within this treatment group all subjects lost at least 5% of their body weight and over half achieved at least 10% body weight loss.
pemvidutide was shown safe and well-tolerated, without dose-titration in the Phase 1a Trial. In this study, up to 10.3% mean weight losses were observed, with statistically significant reductions across multiple atherogenic lipid classes as well as in atherogenic small- and medium-sized lipoprotein particle concentrations size were observed. Based on these findings, pemvidutide demonstrates promise as an agent for inducing weight loss, independent of a Type II diabetes diagnosis, and concurrently improving obesity comorbidities, including reducing CV risk factors (e.g., pathogenic serum lipid mediators).

### Example 3. A Phase 1b, 24-Week, Randomized, Double-Blind, Placebo-Controlled Study of ALT-801 (Pemvidutide) in Diabetic and Non-Diabetic Overweight and Obese Subjects with Non-Alcoholic Fatty Liver Disease (NAFLD)

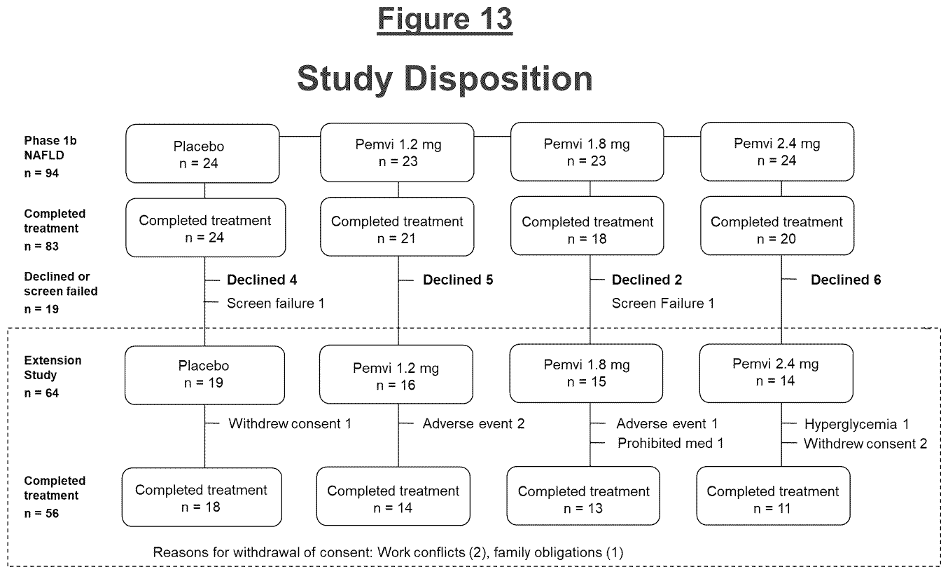

Provided herein is disclosure of a 12-week extension study of the study provided in Example 1, providing a 24-week a Phase 1b, randomized, double-blind, placebo-controlled study to assess the effects of pemvidutide (also termed herein as ALT-801), the safety of pemvidutide and effects on weight loss, hepatic fat fraction, anthropometric parameters, lipid metabolism, as well as inflammatory, fibrosis and other markers in diabetic and non-diabetic (type 2 diabetes) overweight and obese (BMI≥28.0 kg/m²) subjects with NAFLD (defined as liver fat content (LFC) by MRI-PDFF ≥10%; absence of significant fibrosis, defined as FibroScan® LSM ≤10 kPa), alanine aminotransferase (ALT) and aspartate aminotransferase (AST) laboratory values of ≤75 IU/L and HbA1c <9.5. Men and women aged 18-65 years were studied. Diabetic subjects were defined as those that have undergone stable dose (≥3 months) metformin or SLGT-2 therapy and no use of insulin, sulfonylureas, DPP-4, GLP-1 treatment; Ninety-four (94) subjects were enrolled in the initial 12-week Phase 1b NAFLD trial and 83 subjects who completed were invited to receive a total of 12 additional weeks of blinded treatment. Of those subjects, 66 consented to rollover, of which 64 were eligible to participate. The study disposition is shown in FIG. 13.

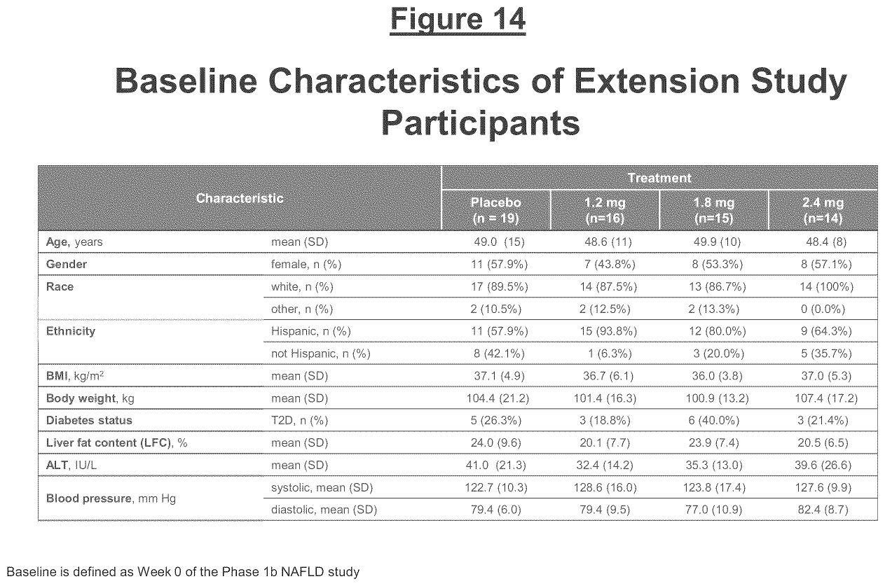

The primary endpoint of this study was a reduction in liver fat content (LFC) by MRI-PDFF at Week 24 compared to Week 0 (zero). Key secondary endpoints include percent (%) weight loss at Week 24 compared to Week 0 (zero), and liver inflammation by alanine aminotransferase (ALT) levels and corrected T1 (cT1) imaging at Week 24 as compared to Week 0 (zero). Adverse events (AEs), including serious and severe AEs, AEs leading to discontinuation, GI tolerability, vital signs, and glycemic control including fasting glucose and HbA1c levels were also measured. Detailed baseline characteristics of the participants in this trial are shown in FIG. 14.

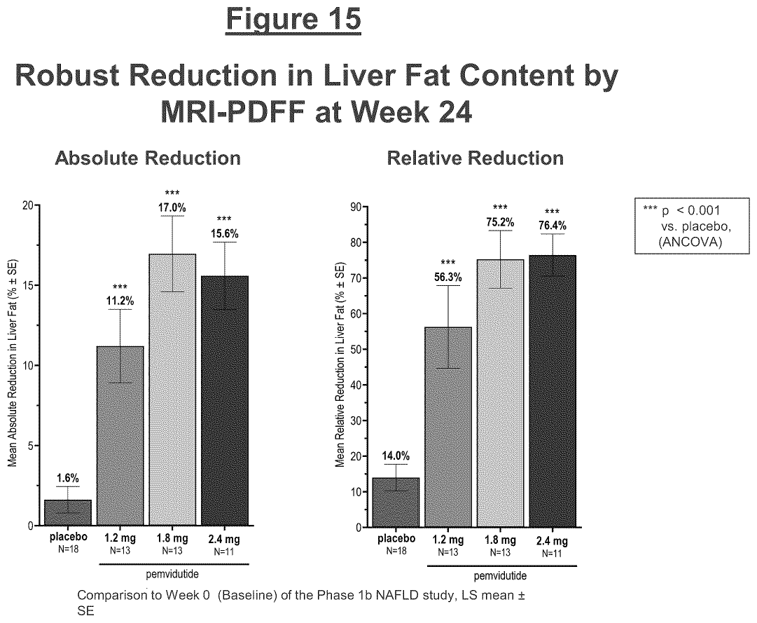

As shown in FIG. 15, a robust reduction in liver fat content as determined by MRI-PDFF at week 24 was observed. As shown therein, significant (p<0.001) reductions in liver fat content for all doses of pemvidutide administered (1.2 mg, 1.8 mg, and 2.4 mg per week) as compared to placebo were observed in both in absolute and relative terms. In terms of absolute reduction, the 1.2, 1.8 and 2.4 mg doses showed an 11.2%, 17% and 15.6% mean absolute reductions in liver fat (p<0.001), respectively. In terms of relative reduction, the 1.2, 1.8 and 2.4 mg doses showed a 56.3, 75.2%, and 76.4% mean relative reductions in liver fat (p<0.001), respectively.

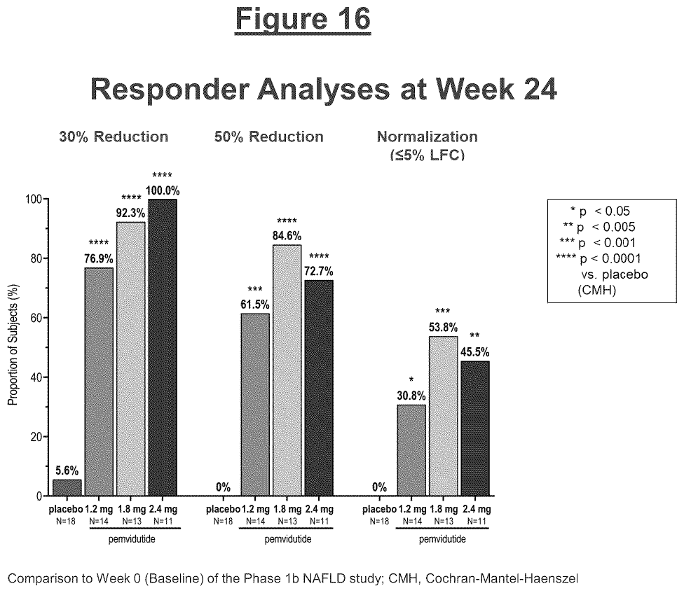

FIG. 16 further also shows the percentage of subjects achieving at least 30% and 50% reduction in liver fat content as well as normalization (≤5%) in liver fat content at Week 24. As shown therein, the percentage of subjects achieving at least 30% and 50% reduction in liver fat content was highly significant (p<0.001 or p<0.0001 as shown therein) for all doses of pemvidutide administered (1.2 mg, 1.8 mg, and 2.4 mg per week) as compared to placebo. In addition, normalization in liver fat content, was achieved in a high proportion of subjects especially at the 1.8 mg dose (53.8%) and the 2.4 mg dose (45.3%), and was highly significant (p<0.001 or p<0.01) for the 1.8 mg and 2.4 mg doses compared to placebo.

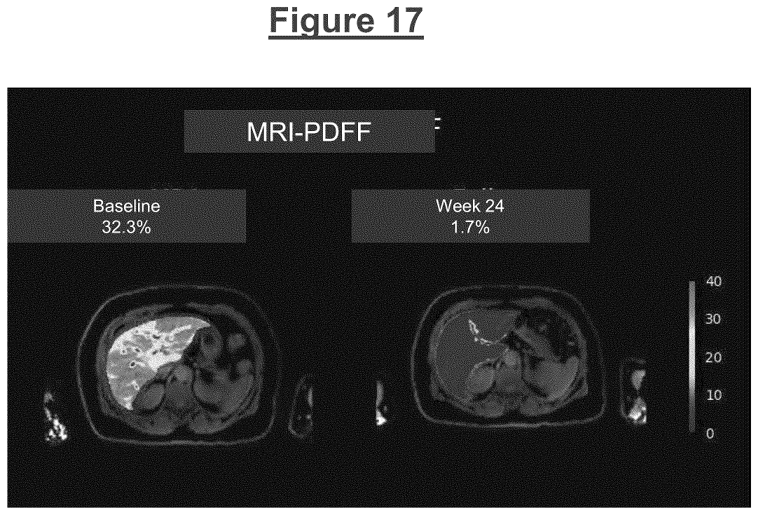

FIG. 17 shows marked defatting of the liver as determined by MRI-PDFF at Week 24 (pemvidutide, 1.8 mg dose). The reduction in liver volume in this scan of the exemplary subject to whom pemvidutide was administered was significant (32.3% at baseline as compared to 1.7% at Week 24).

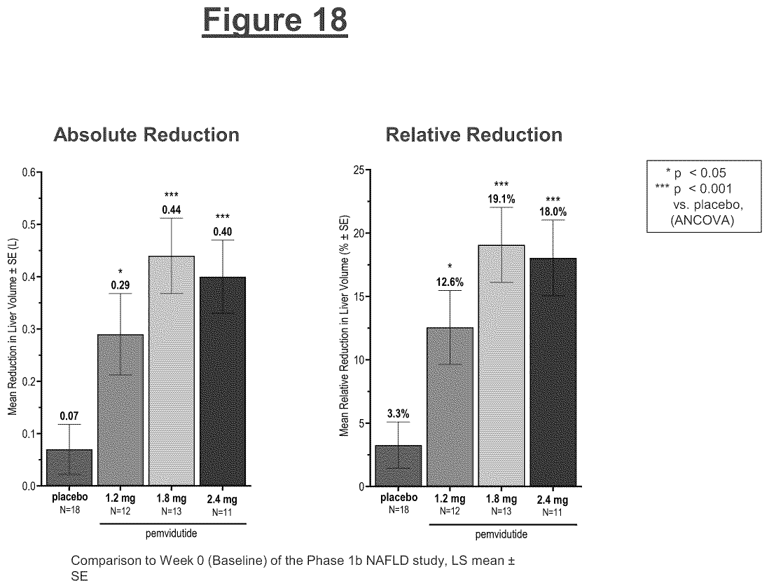

FIG. 18 shows a robust reduction in liver volume as determined by MRI-PDFF at Week 24, measured as both absolute reduction and relative reduction. As shown therein, significant (p<0.05 or p<0.001 as indicated therein) reductions in liver fat content for all doses of pemvidutide administered (1.2 mg, 1.8 mg, and 2.4 mg per week) as compared to placebo were observed in both in absolute and relative terms.

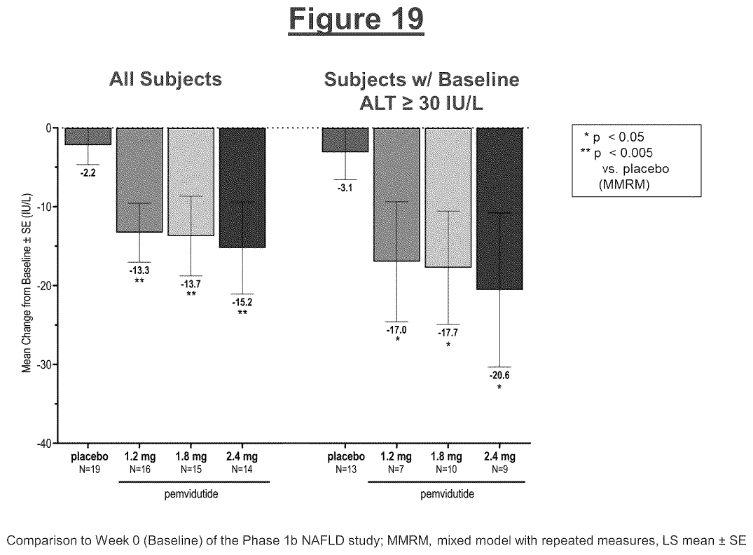

FIG. 19 shows a robust reduction of ALT levels, a biomarker of liver inflammation, at Week 24, in all subjects, which was even more pronounced in subjects with a baseline ALT ≥30 IU/L. As shown therein, e.g., significant (p<0.001) reductions in ALT for all doses of pemvidutide administered (1.2 mg, 1.8 mg, and 2.4 mg per week) as compared to placebo were observed in all subjects and subjects with a baseline ALT ≥30 IU/L.

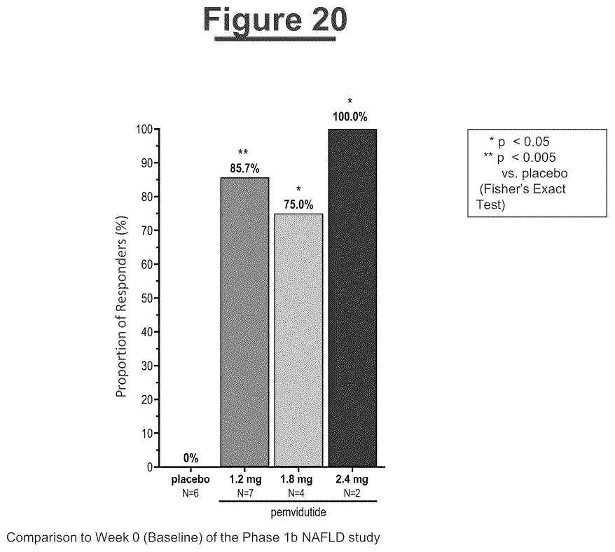

FIG. 20 shows high rates of cT1 response at Week 24. The response threshold is defined as an 80 milliseconds (ms) reduction in CT1 as compared to baseline. As shown therein, e.g., significant (p<0.05 or p<0.005 as indicated therein) cT1 responses for all doses of pemvidutide administered (1.2 mg, 1.8 mg, and 2.4 mg per week) as compared to placebo were observed. It is noted that 80 ms reduction in cT1 approximately has been associated with a 2-point reduction of NASH Activity Score (NAS) (Dennis, A., Front. Endocrinology, 2021) and that elevated cT1 levels have been associated with increased risk of major adverse cardiac events (MACE) and major adverse liver outcomes (MALO) (Jayaswal, A. Liver Int., 2020; Roca-Fernandez A., MedRxiv, 2022).

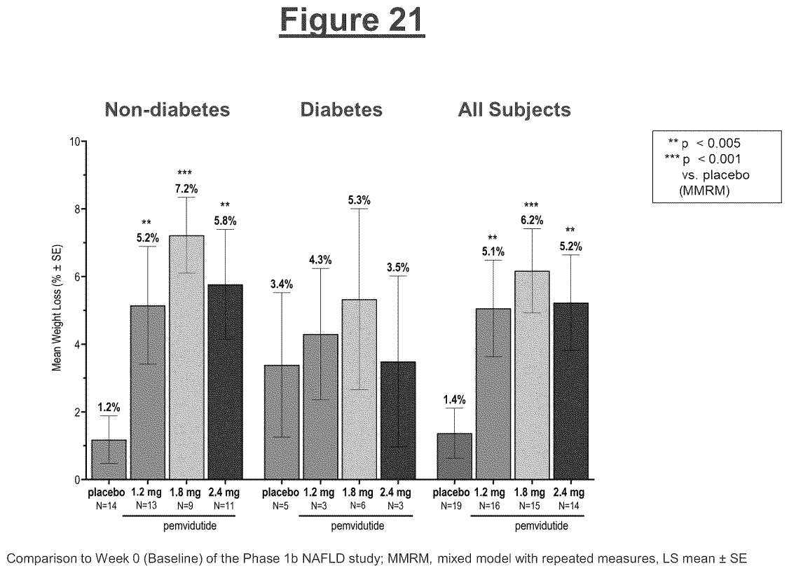

FIG. 21 shows continued weight loss at Week 24 compared to Week 12. This differentiates pemvidutide from other NASH drugs with comparable levels of liver fat reduction. As shown therein, significant (p<0.005 or p<0.001 as indicated therein) reductions in weight for all doses of pemvidutide administered (1.2 mg, 1.8 mg, and 2.4 mg per week) as compared to placebo were observed in non-diabetic patients as well as for all subjects. Mean weight loss was also observed in diabetic patients (see the “Diabetes” panel of FIG. 21).

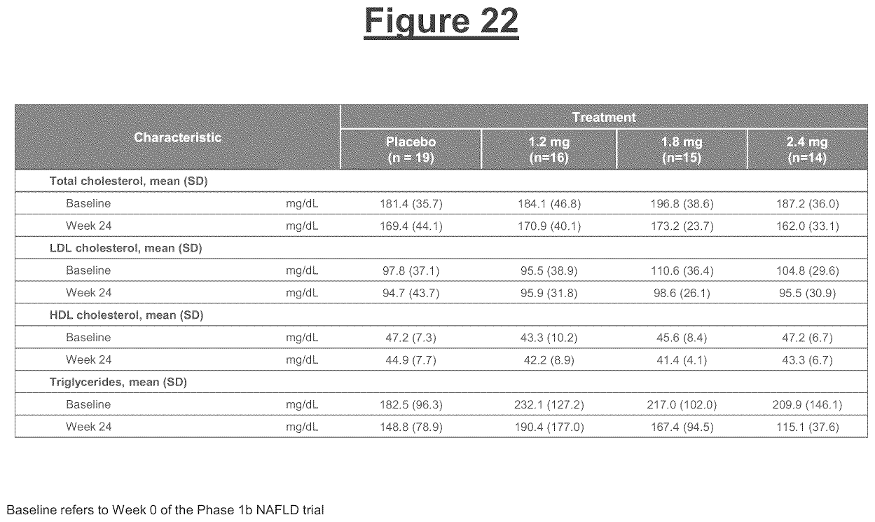

FIG. 22 shows improvements in serum lipids at Week 24. As shown therein, e.g., improvements in serum lipids for all doses of pemvidutide administered (1.2 mg, 1.8 mg, and 2.4 mg per week) were observed.

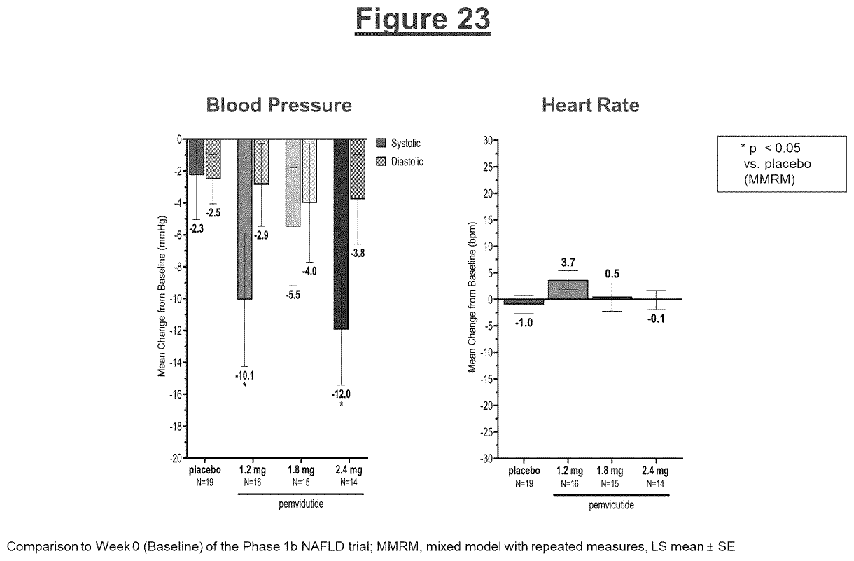

FIG. 23 shows all doses of pemvidutide tested induced improvements in blood pressure without clinically meaningful increases in heart rate at Week 24. As shown therein, e.g., such improvements in blood pressure for all doses of pemvidutide administered (1.2 mg, 1.8 mg, and 2.4 mg per week) were observed. It is noted that systolic blood pressure reduction was significant (p<0.05) at the 2.4 mg per week dose.

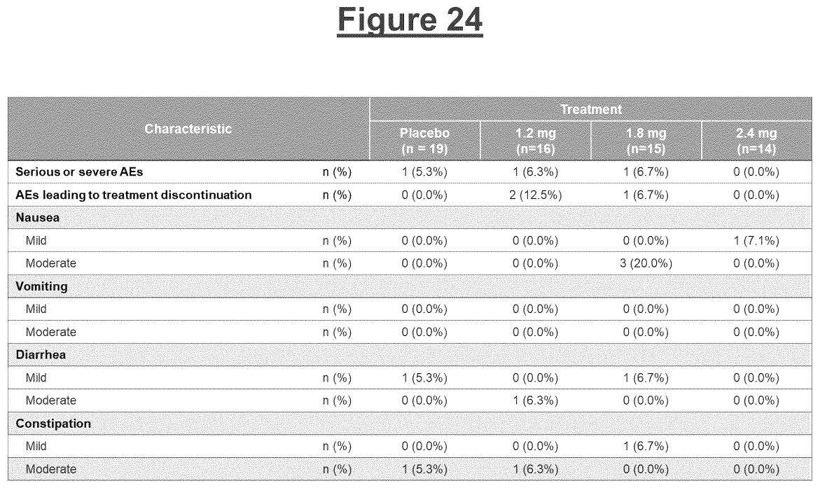

FIG. 24 shows the safety overview of this trial. In the treatment groups, the number of serious or severe adverse events were low and comparable to the placebo group. The serious and severe AEs were the same events and included: 1) chest pain post elective coronary stent placement (placebo), 2) Salmonella infection (pemvidutide, 1.2 mg), and 3) hypertension >3 weeks post last dose of study medication (pemvidutide, 1.8 mg), all unrelated to study medication, with only a Salmonella infection leading to treatment discontinuation. The two (2) gastrointestinal (GI) AEs leading to treatment discontinuation were mild (Grade 1) abdominal pain in two (2) subjects. No significant ALT elevations were reported. In addition, the number of adverse events leading to treatment discontinuation as well as gastrointestinal-related adverse events leading to treatment discontinuation were low across the treatment group. Gastrointestinal-related adverse events such as nausea, vomiting, diarrhea and constipation were mild to moderate.

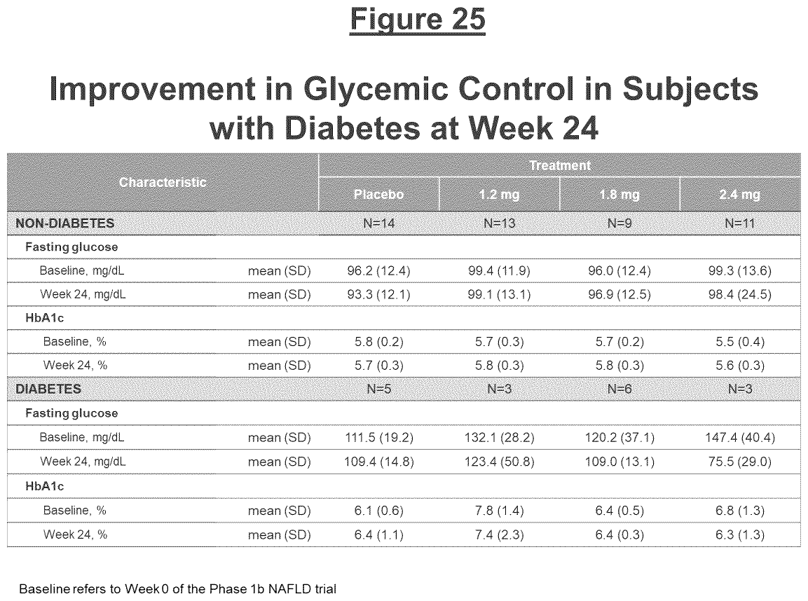

FIG. 25 shows improvements in glycemic parameters (fasting glucose and HbA1c) in diabetic patients as well as maintenance of glycemic control in non-diabetic at Week 24. As shown therein, e.g., such improvements in glycemic control including a reduction in fasting glucose and HbA1c for all doses of pemvidutide administered (1.2 mg, 1.8 mg, and 2.4 mg per week) in diabetic patients were observed. These clinical effects are in support of the antihyperglycemic role of pemvidutide, presumably related to its GLP-1 activity that counteracts the hyperglycemic effect of its glucagon activity.

This study shows that pemvidutide induces liver fat reduction including greater than 75% relative liver fat reductions at 24 weeks (better than or equal to the effects of other leading NASH candidates) and significant reductions in cT1 and serum ALT point to potent effects in NASH clinical trials. This study also shows that pemvidutide induces weight loss in non-diabetic subjects (continued weight loss, achieving 7.2% at Week 24) and diabetic subjects (5.3% weight loss at Week 24). This study also shows that pemvidutide is safe and tolerable (e.g., low rates of AEs leading to treatment discontinuation, no serious/severe AEs related to pemvidutide; well-tolerated without the need for dose titration, consistent with prior experience; no clinically significant ALT elevations; and glycemic control was maintained, with diabetics realizing reduced fasting glucose and HbA1c).

### Example 4: Efficacy Interim Analysis of a Phase II, 24-Week, Randomized, Double-Blind, Placebo-Controlled Study of ALT-801 (Pemvidutide) in Non-Diabetic Obese and Overweight Subjects with Higher Risk of NAFLD and NASH

Results presented in this example correspond to a 24-week interim analysis obtained from a placebo-controlled phase II study (ClinicalTrials.gov Identifier: NCT05295875) evaluating pemvidutide (ALT-801) in obese and overweight subjects. It is estimated that 70 to 75% subjects of this population have NAFLD while 34% may have NASH (Quek J. et al. Global prevalence of non-alcoholic fatty liver disease and non-alcoholic steatohepatitis in the overweight and obese population: a systematic review and meta-analysis. Lancet Gastroenterol Hepatol. 2023 January; 8(1):20-30). Key eligibility criteria were: (1) Men and women, ages 18-75 years; (2) at least one unsuccessful weight loss attempt per Investigator judgement; (3) Body Mass Index (BMI) ≥30 kg/m2 or BMI ≥27 kg/m2 with at least one obesity-related comorbidity (history of cardiovascular disease, hypertension, dyslipidemia, pre-diabetes or obstructive sleep apnea) and (4) non-diabetes (HbA1c ≤6.5% and fasting glucose ≤125 mg/dL). Eligible subjects were randomized 1:1:1:1 to one of the following treatment arms: Group 1 (39 subjects): pemvidutide 1.2 mg SC once weekly for 24 weeks; Group 2 (40 subjects): pemvidutide 1.8 mg administered subcutaneously once weekly for 24 weeks; Group 3 (40 subjects): pemvidutide 0.6 mg administered subcutaneously for 1 week, 1.2 mg administered subcutaneously for 1 week, 1.8 mg administered subcutaneously once weekly for 2 weeks, followed by 2.4 mg administered subcutaneously once weekly for 20 additional weeks (in sequence) and Group 4 (41 subjects): Placebo administered subcutaneously once weekly for 24 weeks. The randomization of subjects was stratified based on sex and baseline body mass index (BMI <35 kg/m2 vs. ≥35 kg/m2). A minimum of 25% of randomized subjects were male. All subjects received counselling on a reduced-calorie diet of 1200-1500 calories for individuals <250 lbs (113.6 kg) and 1500-1800 calories for individuals ≥250 lbs (113.6 kg) and a gradual increase in physical activity (targeting 150 min of physical activity per week) by a qualified healthcare professional at screening and at subsequent visits during the treatment period. Subjects were instructed to record their food intake and physical activity daily, and compliance with lifestyle interventions will be assessed by the investigator on a regular basis. A subgroup of subjects were subjected MRI-PDFF to evaluate the hepatic fat fraction in as well as composition to measure total body adipose tissue (AT) and adipose tissue-free mass (ATFM).

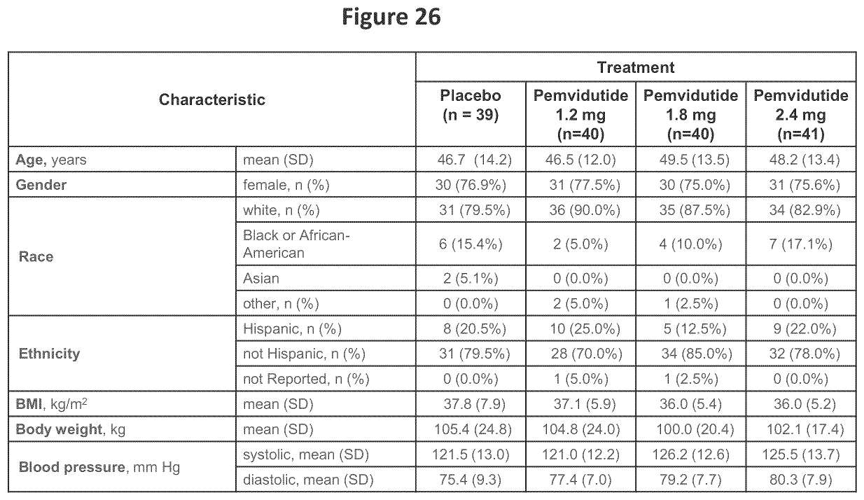

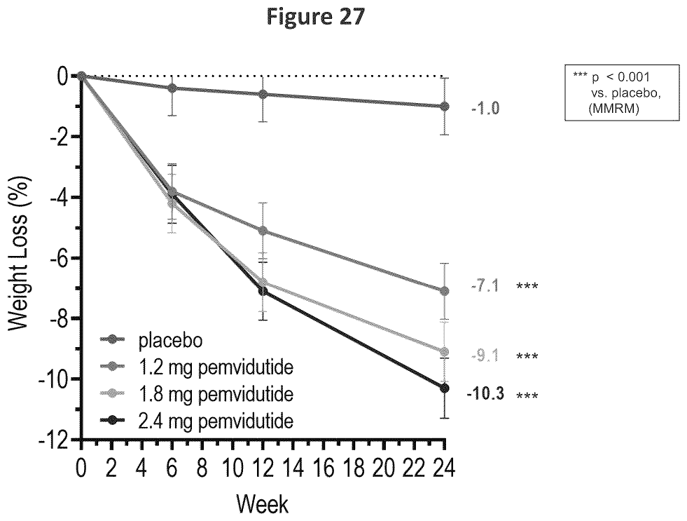

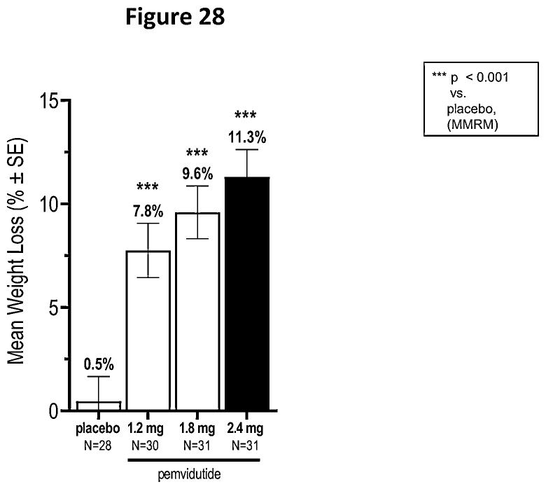

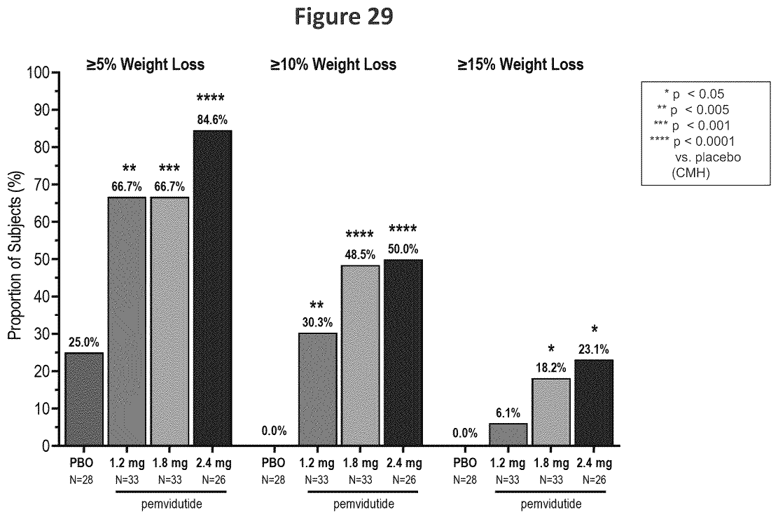

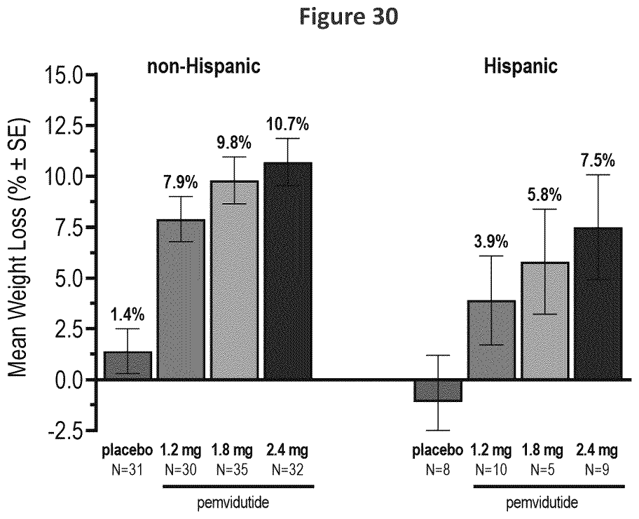

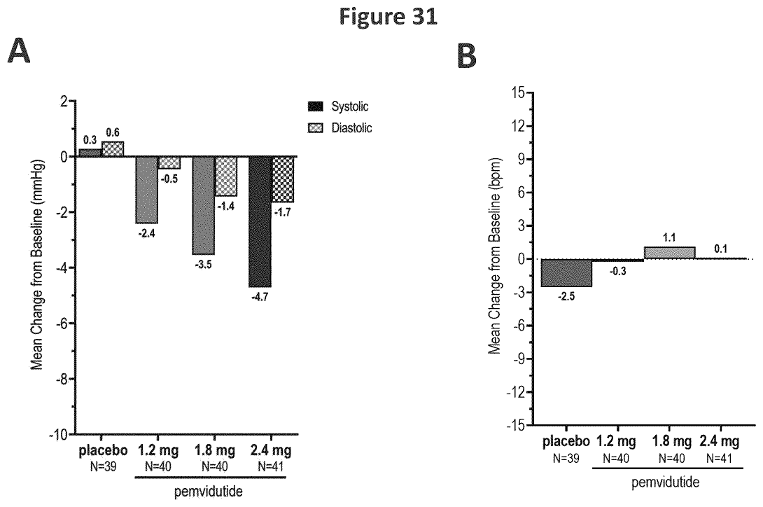

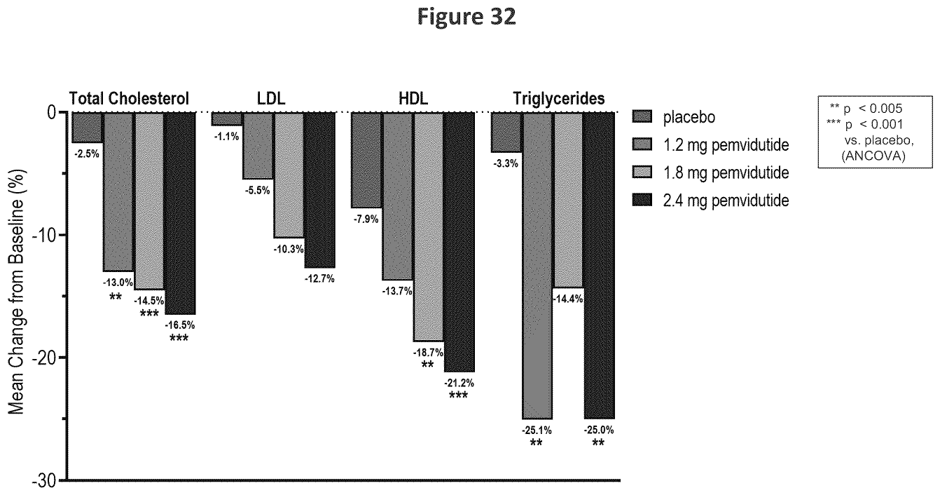

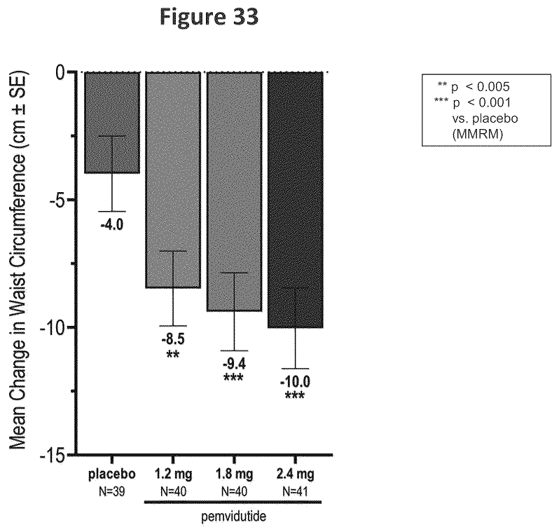

Baseline characteristics of study participants are presented in FIG. 26. FIG. 27 presents the mean percentage weight loss over time in all evaluated subjects across the three pemvidutide doses and placebo over 24 weeks. Results are presented as efficacy estimand that assumes subjects remained on treatment for the entire duration, with missing values handled by Mixed Models for Repeated Measures. FIG. 27 shows a progressive, dose-dependent weight loss achieved with pemvidutide over time as opposed to Placebo. FIG. 28 presents the mean percentage weight loss (presented as efficacy estimand as described above) that was achieved over 24 weeks with pemvidutide and placebo in a subgroup of subjects with baseline body weight ≤115 kg. As for results presented in FIG. 27, FIG. 28 shows a progressive, dose-dependent weight loss achieved with pemvidutide over time as opposed to Placebo. When compared to FIG. 27, results presented in FIG. 28 indicate that higher levels of weight loss is achieved by each of the three doses of pemvidutide in subjects with lower body weight at baseline. This phenomenon may relate to a lower level of drug exposure in subjects with higher body weight, and would support the possibility to increase dose levels in this population to maximize efficacy. FIG. 29 presents a weight loss responder analysis based on calculation of percentage of subjects who respectively achieve ≥5%, ≥10% and ≥15% weight loss across the three pemvidutide doses and placebo. FIG. 30 presents the percentage weight loss achieved across the four groups comparing Hispanic versus non-Hispanic subjects. Results showed overall that subjects with Hispanic decent tend to lose less body weight following treatment with pemvidutide indicative of possible impact of genetic background. FIG. 31A shows that 24-week treatment with pemvidutide improves both systolic and diastolic blood pressures in a dose dependent manner as opposed to placebo. This result is important considering that obesity represent a major cause of hypertension. FIG. 31B shows that 24-week treatment with pemvidutide at 1.2 mg, 1.8 mg and 2.4 mg doses do not induce a significant change in heart rates. FIG. 32 shows serum lipids at week 24 decreased as compared to baseline for all doses of pemvidutide administered (1.2 mg, 1.8 mg, and 2.4 mg). Thus, FIG. 32 shows improvements in serum lipids at week 24 for all doses of pemvidutide administered (1.2 mg, 1.8 mg, and 2.4 mg per week). FIG. 33 shows a significant reduction in waist circumference at week 24 for all doses of pemvidutide administered (1.2 mg, 1.8 mg, and 2.4 mg per week). Waist circumference is an index of central or abdominal obesity recommended by the WHO to assess the risk of metabolic diseases such as NAFLD and NASH and cardiovascular diseases.

Accordingly, provided herein is a method of using pemvidutide for reducing body weight in a human being with a high risk of fatty liver disease, wherein the method comprises administering pemvidutide once weekly in an amount from at least 1.8 mg up to 2.4 mg to the human being in need thereof; and, wherein the human being does not suffer from type 2 diabetes and wherein the fatty liver disease is non-alcoholic fatty liver disease (NAFLD) or non-alcoholic steatohepatitis (NASH).

Other advantages of the reagents and methods of using the same are also provided herein, as would be understood by those of ordinary skill in the art. While certain embodiments have been described in terms of the preferred embodiments, it is understood that variations and modifications will occur to those skilled in the art. Therefore, it is intended that the appended claims cover all such equivalent variations that come within the scope of the following claims.

1. A method of reducing body weight in a human being with fatty liver disease, the method comprising administering pemvidutide once weekly in an amount from at least about 1.2 to about 2.4 mg, to the human being in need thereof for at least about 12; wherein the fatty liver disease is non-alcoholic fatty liver disease (NAFLD) or non-alcoholic steatohepatitis (NASH).

2. The method of claim 1, wherein the body weight of the human being is reduced by at least 3% from baseline at week 12.

3. The method of claim 1, wherein the body weight of the human being is reduced by at least 4% from baseline at week 12.

4. (canceled)

5. (canceled)

6. The method of claim 1, wherein the pemvidutide is administered once weekly in an amount of 2.4 mg.

7. The method of claim 1, wherein a steady state dose is achieved after a dose escalating phase having a duration of about 2 weeks, about 3 weeks or about 4 weeks.

8. The method of claim 1, wherein the human being has a diagnosis of type 2 diabetes.

9. The method of claim 1, wherein the human being does not have a diagnosis of type 2 diabetes.

10. The method of claim 1, wherein the human being has a body mass index (BMI kg/m²) of at least 28.

11. The method of claim 1, wherein the human being has a body mass index (BMI kg/m²) of 30 or greater.

12. The method of claim 1, wherein the human being has a level of liver fat as measured by MRI-PDFF of 10% or greater.

13. The method of claim 1, wherein an absolute reduction in liver fat as measured by MRI-PDFF is a reduction of about 8% to about 15% after 12 weeks of the once weekly dosing.

14. The method of claim 1, wherein the relative reduction in liver fat as measured by MRI-PDFF compared to baseline is about 40% to about 70% after 12 weeks of the once weekly dosing.

15. The method of claim 1, wherein the relative reduction in liver fat as measured by MRI-PDFF compared to baseline is about 30% to about 50% after 24 weeks of the once weekly dosing, wherein the relative reduction is statistically significant defined as p<0.001 or p<0.0001.

16. (canceled)

17. The method of claim 1, wherein defatting of the liver as measured by MRI-PDFF compared to baseline is about 30% after 24 weeks of once weekly dosing of about 1.8 mg pemvidutide as compared to baseline in the human being.

18. (canceled)

19. The method of claim 1, wherein alanine aminotransferase (ALT) is reduced after 24 weeks of once weekly dosing as compared to baseline, wherein the human being has baseline ALT of >30 IU/L before dosing.

20-40. (canceled)

41. A method of inducing weight loss in a human being with fatty liver disease, the method comprising administering pemvidutide once weekly for at least 12 continuous weeks in an amount from at least about 1.2 mg up to about 2.4 mg to the human being, wherein the human being:
has been diagnosed with fatty liver disease that is non-alcoholic fatty liver disease (NAFLD) or non-alcoholic steatohepatitis (NASH);
has a body mass index (BMI kg/m²) of greater than about 28; and,

42-66. (canceled)

67. The method of claim 1, wherein the pemvidutide is administered from a liquid formulation comprising at least about 2.5 mg/ml pemvidutide.

68-72. (canceled)

73. A liquid pharmaceutical formulation comprising: SEQ ID NO: 1 and about 0.020% (w/w) polysorbate 20, about 0.348% (w/w) arginine, and about 4.260% (w/w) mannitol in deionized water (pH 7.7±0.1).

74. The formulation of claim 73, comprising 1.8 mg of SEQ ID NO: 1 as a therapeutic dose.

75. The formulation of claim 74, wherein the therapeutic dose induces weight loss in a subject in need thereof.
